(19) United States
(12) Patent Application Publication
(10) Pub. No.: US 2006/0223708 A1
(43) Pub. Date: Oct. 5, 2006

(54) Substituted N-[pyrimidin-2-ylmethyl]carboxamides and their use as herbicides and plant growth regulators

(75) Inventors: Michael Gerhard Hoffmann, Florsheim (DE); Klaus Haaf, Kelkheim (DE); Hendrik Helmke, Liederbach (DE); Lothar Willms, Hofheim (DE); Thomas Auler, Leichlingen (DE); Martin Hills, Idstein (DE); Heinz Kehne, Hofheim (DE); Dieter Feucht, Eschborn (DE)

Correspondence Address: CONNOLLY BOVE LODGE & HUTZ, LLP, P O BOX 2207, WILMINGTON, DE, 19899, US

(73) Assignee: Bayer CropScience GmbH, Frankfurt am Main (DE)

(21) Appl. No.: 11/395,928

(22) Filed: Mar. 31, 2006

(30) Foreign Application Priority Data
Apr. 2, 2005 (DE) 10 2005 014 906.5

(51) Int. Cl. A01N 43/54; C07D 405/02; C07D 403/02

(52) U.S. Cl. 504/242; 544/314

(57) ABSTRACT

What is described are N-[pyrimidin-2-ylmethyl]carboxamides of the formula (I) and their use as herbicides. 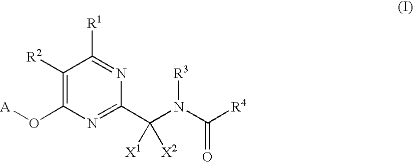
In this formula (I), X¹ and X² are hydrogen or methyl, R¹ to R⁴ are various radicals and A is an aromatic or heteroaromatic ring.

The present invention relates to novel herbicidally active N-[pyrimidin-2-yl-methyl]carboxamide derivatives, to processes for their preparation and to their use as herbicides and plant growth regulators, in particular for the selective control of broad-leaved weeds and wheat grasses in crops of useful plants.

From various publications, it is already known that certain prymidines substituted by azole radicals, such as pyrazolyl, imidazolyl and triazolyl, have herbicidal properties, see, for example, WO 98/40379, WO 98/56789, WO 99/28301, WO 00/63183, WO 0.1/90080, WO 03/016308 and WO 03/084331. However, when using the compounds known from these publications, there are in some cases disadvantages, such as, for example, high persistency, insufficient selectivity in important crops of useful plants or excessive application rates.

Substituted N-[pyrimidin-2-ylmethyl]carboxamides are known from some publications, see, for example, J. Med. Chem., 2002, 143-150; Synth. Commun., 2002, 153-158; Chem. Pharm. Bull., 1983, 2540-2551; Vestn. Mosk. Univ. Ser. 2 Khim., 17, 1962, 70; Chem. Abstr. 58, 521c, 1963. However, these publications do not disclose any herbicidal action of such compounds.

It is an object of the present invention to provide herbicidally active compounds having herbicidal properties which are improved—improved, that is, over those of the prior art compounds—and having improved compatibility with crop plants.

It has now been found that certain substituted N-[pyrimidin-2-ylmethyl]carboxamides have good herbicidal action and, at the same time, are highly compatible with useful plants. Accordingly, the present invention provides compounds of the formula (I), their N-oxides and/or their salts 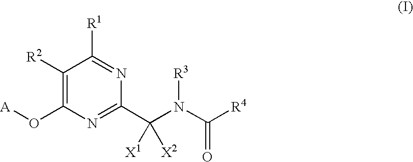
in which the radicals and indices are as defined below:
R¹ and R² independently of one another are hydrogen, halogen, cyano, amino, isocyanato, hydroxyl, nitro, COOR⁵, COR⁵, CH₂OH, CH₂SH, CH₂NH₂, (C₁-C₄)-alkyl, halo-(C₁-C₄)-alkyl, (C₃-C₆)-cycloalkyl, (C₁-C₄)-alkoxy, halo-(C₁-C₄)-alkoxy, (C₁-C₂)-alkoxy-(C₁-C₂)-alkyl, (C₂-C₄)-alkenyl, (C₂-C₄)-alkynyl, (C₃-C₄)-alkenyloxy, (C₃-C₄)-alkynyloxy, (C₁-C₂)-alkylthio-(C₁-C₂)-alkyl, S(O)NR⁶, (C₁-C₂)-alkylsulfonyl-(C₁-C₂)-alkyl, (C₁-C₄)-alkyl-NH, (C₁-C₃)-alkyl-CO—NH, (C₁-C₄)-alkyl-SO₂NH, di-(C₁-C₄)-alkylamino,
   or R¹ and R² together form the group (CH₂)₃;
R³ is hydrogen, (C₁-C₄)-alkyl, (C₂-C₄)-alkenyl, (C₂-C₄)-alkynyl, benzyl, COOR⁵, COR⁴ or S(O)ₙR⁶;
R⁴ is hydrogen, (C₁-C₈)-alkyl, (C₂-C₈)-alkenyl, (C₂-C₈)-alkynyl, (C₃-C₆)-cycloalkyl, (C₃-C₆)-cycloalkyl which is substituted by one or two methyl groups, (C₁-C₂)-alkoxy-(C₁-C₂)-alkyl, (C₃-C₆)-cycloalkyl-(C₁-C₂)-alkyl, halo-(C₁-C₆)-alkyl or halo-(C₃-C₆)-cycloalkyl;
R⁵ is hydrogen or (C₁-C₄)-alkyl;
R⁶ is hydrogen, (C₁-C₄)-alkyl or halo-(C₁-C₄)-alkyl;
A is a radical from the group comprising the substituents A1 to A8 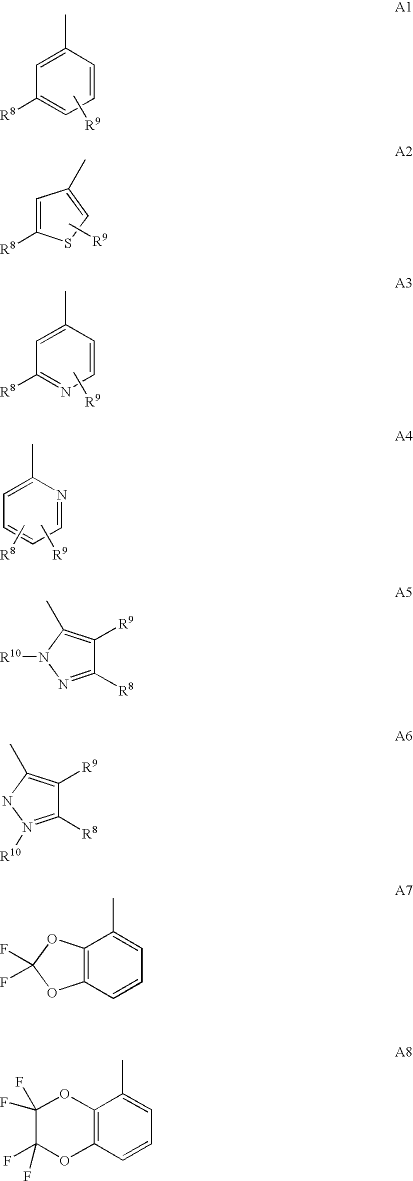
R⁸ is hydrogen, halogen, cyano, isocyanato, nitro, (C₁-C₄)-alkyl, halo-(C₁-C₄)-alkyl, (C₁-C₄)-alkoxy, halo-(C₁-C₄)-alkoxy, halo-(C₁-C₄)-alkylthio, (C₃-C₆)-cycloalkyl, halo-(C₃-C₆)-cycloalkyl, SF₅, S(O)ₙR⁶, (C₂-C₄)-alkenyl or (C₂-C₄)-alkynyl;
R⁹ is hydrogen, halogen, cyano, isocyanato, nitro, (C₁-C₄)-alkyl, halo-(C₁-C₄)-alkyl, (C₁-C₄)-alkoxy, halo-(C₁-C₄)-alkoxy, (C₂-C₄)-alkenyl, (C₂-C₄)-alkynyl, (C₃-C₆)-cycloalkyl or S(O)ₙR⁶;
R¹⁰ is (C₁-C₄)-alkyl;
X¹, X² independently of one another are hydrogen or (C₁-C₄)-alkyl;
n is 0, 1 or 2.

In formula (I) and all subsequent formulae it is possible for alkyl radicals having more than two carbon atoms to be straight-chain or branched. Alkyl radicals are, for example, methyl, ethyl, n- or i-propyl, n-, i-, tert- or 2-butyl, pentyls, hexyls, such as n-hexyl, i-hexyl and 1,3-dimethylbutyl. This applies analogously to the unsaturated radicals alkenyl and alkynyl. Cycloalkyl is cyclopropyl, cyclobutyl, cyclopentyl and cyclohexyl.

Halogen is fluorine, chlorine, bromine or iodine.

In unsaturated radicals, such as alkenyl und alkynyl, the multiple bond may be in any position of the radical. Thus, for example, the radical propynyl may be 1-propynyl or 2-propynyl.

Where a group is substituted more than once by radicals this means that this group is substituted by one or more identical or different radicals from among those specified.

Depending on the nature and the attachment of the substituents, the compounds of the formula (I) may be present in the form of stereoisomers. Where, for example, there are one or more asymmetric carbon atoms present, enantiomers and diastereomers may occur. Stereoisomers can be obtained from the as-prepared mixtures by standard separation methods, such as by chromatographic separation methods, for example. Likewise, stereoisomers can be prepared selectively by using stereoselective reactions and employing optically active starting materials and/or auxiliaries. The invention also provides all stereoisomers and mixtures thereof that, while embraced by the formula (I), have not been defined specifically.

Preference is given to compounds of the formula (I) in which
R¹ and R² independently of one another are hydrogen, halogen, cyano, hydroxyl, nitro, (C₁-C₂)-alkyl, halo-(C₁-C₂)-alkyl, (C₁-C₂)-alkoxy, halo-(C₁-C₂)-alkoxy, (C₁-C₂)-alkoxy-(C₁-C₂)-alkyl, (C₁-C₂)-alkylthio-(C₁-C₂)-alkyl, S(O)ₙ—(C₁-C₂)-alkyl,
   or R¹ and R² together form the group (CH₂)₃;
R³ is hydrogen, (C₁-C₂)-alkyl, benzyl or COR⁴;
R⁴ is hydrogen, (C₁-C₆)-alkyl, (C₂-C₄)-alkenyl, (C₂-C₄)-alkynyl, (C₃-C₆)-cycloalkyl, (C₃-C₆)-cycloalkyl which is substituted by a methyl group, (C₁-C₂)-alkoxy-(C₁-C₂)-alkyl, (C₃-C₆)-cycloalkyl-(C₁-C₂)-alkyl, halo-(C₁-C₄)-alkyl or halo-(C₃-C₆)-cycloalkyl;
R⁵ is hydrogen or (C₁-C₄)-alkyl;
R⁶ is hydrogen, (C₁-C₂)-alkyl or halo-(C₁-C₂)-alkyl;
A is a radical from the group comprising the substituents A1 to A8;
R⁸ is hydrogen, halogen, cyano, (C₁-C₂)-alkyl, halo-(C₁-C₂)-alkyl, (C₁-C₂)-alkoxy, halo-(C₁-C₂)-alkoxy, halo-(C₁-C₂)-alkylthio, (C₃-C₆)-cycloalkyl, halo-(C₃-C₆)-cycloalkyl, S(O)ₙR⁶, (C₂-C₄)-alkenyl or (C₂-C₄)-alkynyl;
R⁹ is hydrogen, halogen, cyano, nitro, (C₁-C₂)-alkyl, halo-(C₁-C₂)-alkyl, (C₁-C₂)-alkoxy, halo-(C₁-C₂)-alkoxy, (C₂-C₂)-alkenyl, (C₂-C₄)-alkynyl, (C₃-C₆)-cycloalkyl or S(O)NR⁶;
R¹⁰ is methyl or ethyl;
X¹, X² independently of one another are hydrogen or methyl;
n is 0, 1 or 2.

Particular preference is given to compounds of the formula (I) in which
R¹ and R² independently of one another are hydrogen, halogen, cyano, methyl, ethyl, trifluoromethyl, difluoromethyl, methoxy, trifluoromethoxy, difluoromethoxy, ethoxymethyl, methoxymethyl, thiomethyl, methylsulfonyl, or R¹ and R² together form the group (CH₂)₃;
R³ is hydrogen, methyl, ethyl or COR⁴;
R⁴ is hydrogen, (C₁-C₄)-alkyl, (C₂-C₄)-alkenyl, (C₂-C₄)-alkynyl, (C₃-C₆)-cycloalkyl, cyclopropyl which is substituted by a methyl group, (C₁-C₂)-alkoxy-(C₁-C₂)-alkyl, (C₃-C₆)-cycloalkyl-(C₁-C₂)-alkyl, halo-(C₁-C₄)-alkyl or halo-(C₃-C₆)-cycloalkyl;
R⁵ is hydrogen or (C₁-C₄)-alkyl;
R⁶ is hydrogen, methyl or ethyl;
A is a radical from the group comprising the substituents A1 to A6;
R⁸ is hydrogen, halogen, cyano, methyl, ethyl, halo-(C₁-C₂)-alkyl, (C₁-C₂)-alkoxy, halomethoxy, (C₃-C₆)-cycloalkyl or S(O)ₙR⁶; very particularly preferably trifluoromethyl, difluoromethyl, trifluoromethoxy, difluoromethoxy or chlorine;
R⁹ is hydrogen, halogen, cyano, nitro, methyl, ethyl, halo-(C₁-C₂)-alkyl, (C₁-C₂)-alkoxy, halomethoxy, (C₂-C₂)-alkenyl, (C₂-C₄)-alkynyl, (C₃-C₆)-cycloalkyl or S(O)NR⁶;
R¹⁰ is methyl or ethyl;
X¹, X² are hydrogen;
n is 0 or 2.

Particular preference is also given to compounds of the formula (I) according to the invention and salts thereof containing a combination of radicals from the preferred compounds mentioned above, and to those containing individual or a number of radicals from the compounds listed in Tables 1 to 6 of the present description.

In all formulae specified below, the substituents and symbols, unless defined otherwise, have the same meaning as described under formula (I).

The compounds of the formula I according to invention and the starting materials and intermediates required for them can be prepared according to the methods described below.

Compounds of the formula I can be prepared, for example, according to Scheme 1 from compounds of the formula II in which E is a leaving group, such as halogen, methylsulfonyl or tosyl, or by reaction with a hydroxyl compound of the formula III in the presence of a base. A is in each case one of the radicals A1 to A8. Such reactions are known to the person skilled in the art.

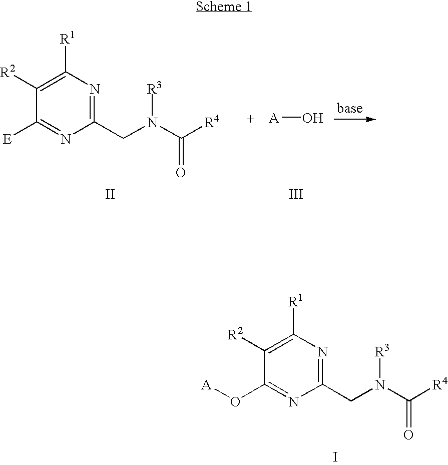

Compounds of the formula II in which E is methylsulfonyl can be prepared, for example, according to Scheme 2 from compounds of the formula IV by oxidation with m-chloroperbenzoic acid (MC PA).

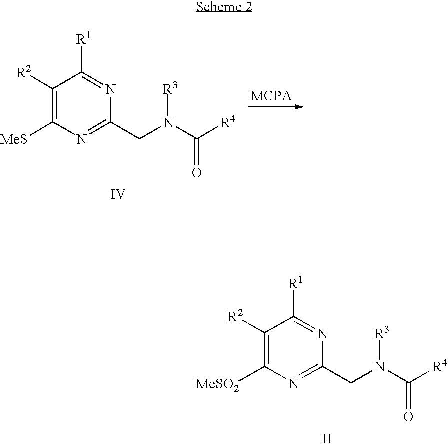

Compounds of the formula IV in which R³ is H can be prepared, for example, according to Scheme 3 by base-induced reaction of a compound of the formula V with carbonyl chlorides. These compounds of the formula IV can then be converted into compounds of the formula IVa in which R³ is an acyl radical (COR⁴), for example by a further base-induced acylation reaction. Such acylation reactions are known to the person skilled in the art.

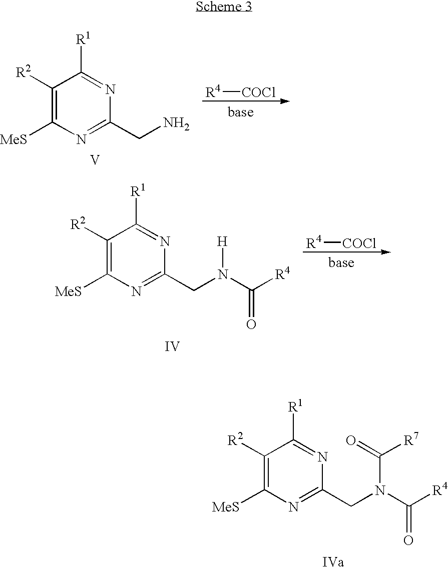

The compounds of the formula V can be prepared, for example, according to Scheme 4 by reducing the corresponding 2-azidomethylpyrimidines of the formula VI with hydrogen sulfide. 2-azidomethylpyrimidines of the formula VI can be synthesized, for example, directly from the corresponding 2-hydroxymethyl-pyrimidines of the formula VII by base-catalyzed reaction with diphenyl phosphoryl azide. 2-hydroxymethylpyrimidine of the formula VII can be obtained, for example, from the corresponding 2-methoxymethylpyrimidines of the formula VIII by ether cleavage using boron trichloride. The reactions shown in Scheme 4 are known to the person skilled in the art.

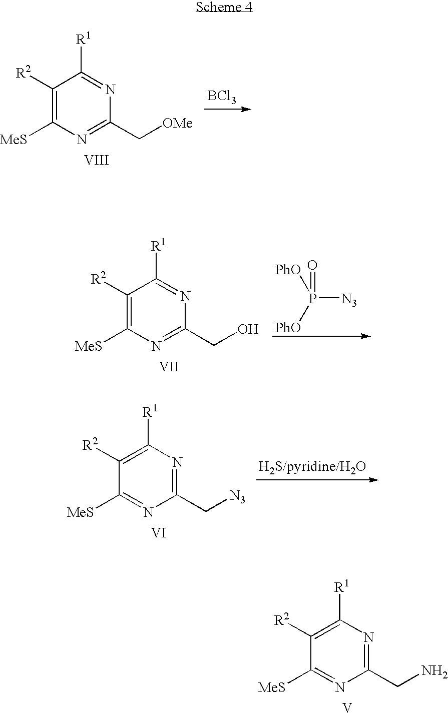

According to Scheme 5, it is possible to prepare 4-methylthiopyrimidines of the formula VIII from 4-chloropyrimidines of the formula IX by base-induced reactions with thiomethanol. The chloropyrimidines of the formula IX can be obtained from hydroxypyrimidines of the formula X by reactions with halogenating agents, such as thionyl chloride, phosgene, phosphorus oxychloride or phosphorus pentachloride. The hydroxypyrimidines of the formula X (where R¹=alkyl) can be prepared by β-keto esters of the formula XI by condensation reactions with methoxymethyl-amidine.

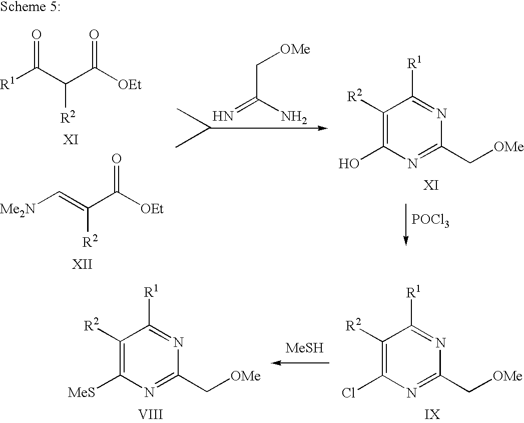

The compounds of the formula (I) according to the invention have an excellent herbicidal activity against a broad spectrum of economically important monocotyledonous and dicotyledonous weed plants. The active substances provide effective control even of perennial weeds which produce shoots from rhizomes, root stocks or other perennial organs and which cannot be easily controlled. In this context, it generally does not matter whether the substances are applied before sowing, pre-emergence or post-emergence. Some representatives of the monocotyledonous and dicotyledonous weed flora which can be controlled by the compounds according to the invention may be mentioned individually as examples, but this is not to be taken to mean a restriction to certain species. The monocotyledonous weed species which are controlled well are, for example, Avena, Lolium, Alopecurus, Phalaris, Echinochloa, Digitaria, Setaria and Cyperus species from the annual group, and Agropyron, Cynodon, Imperata and Sorghum or else perennial Cyperus species amongst the perennial species. In the case of dicotyledonous weed species, the spectrum of action extends to species such as, for example, Galium, Viola, Veronica, Lamium, Stellaria, Amaranthus, Sinapis, lpomoea, Sida, Matricaria and Abutilon from the annual group, and Convolvulus, Cirsium, Rumex and Artemisia among the perennial weeds. Weed plants which are found under the specific culture conditions of rice, such as, for example, Echinochloa, Sagittaria, Alisma, Eleocharis, Scirpus and Cyperus, are also controlled outstandingly well by the active substances according to the invention. If the compounds according to the invention are applied to the soil surface prior to germination, then either emergence of the weed seedlings is prevented completely, or the weeds grow until they have reached the cotyledon stage but growth then comes to a standstill and, after a period of three to four weeks, the plants eventually die completely. When the active substances are applied post-emergence to the green parts of the plants, growth also stops drastically very soon after the treatment, and the weeds remain at the growth stage of the time of application, or, after a certain period of time, they die completely so that in this way competition by the weeds, which is detrimental for the crop plants, is thus eliminated at a very early stage and in a sustained manner. In particular, the compounds according to the invention have an outstanding action against Apera spica venti, Chenopodium album, Lamium purpureum, Polygonum convulvulus, Stellaria media, Veronica hederifolia, Veronica persica, Viola tricolor and also against Amaranthus, Galium and Kochia species.

The compounds according to the invention have an outstanding herbicidal activity against monocotyledonous and dicotyledonous weeds, and yet crop plants of economically important crops such as, for example, wheat, barley, rye, rice, corn, sugar beet, cotton and soybean suffer only negligible damage, if any. In particular, they are outstandingly well tolerated in corn, rice, cereals and soybean. This is why the present compounds are highly suitable for the selective control of unwanted vegetation in stands of agricultural useful plants or of ornamentals.

Owing to their herbicidal properties, these compounds can also be employed for controlling weed plants in crops of genetically modified plants which are known or are yet to be developed. As a rule, the transgenic plants are distinguished by particularly advantageous properties, for example by resistances to certain pesticides, especially certain herbicides, by resistances to plant diseases or causative organisms of plant diseases, such as certain insects or microorganisms such as fungi, bacteria or viruses. Other particular properties concern for example the harvested material with regard to quantity, quality, shelf life, composition and specific constituents. Thus, transgenic plants are known which have an increased starch content or whose starch quality has been modified, or those whose fatty acid composition in the harvested material is different.

The compounds of the formula (I) according to the invention or their salts are preferably employed in economically important transgenic crops of useful plants and ornamentals, for example cereals such as wheat, barley, rye, oats, millet, rice, cassaya and corn, or else crops of sugar beet, cotton, soybean, oilseed rape, potato, tomato, pea and other vegetables. The compounds of the formula (I) can preferably be employed as herbicides in crops of useful plants which are resistant, or have been genetically modified to be resistant, to the phytotoxic effects of the herbicides, in particular soybean and corn.

Conventional routes for the generation of novel plants which have modified properties compared with existing plants are, for example, traditional breeding methods and the generation of mutants. Alternatively, novel plants with modified properties can be generated with the aid of recombinant methods (see, for example, EP-A-0221044, EP-A-0131624). For example, several cases of the following have been described:
recombinant modifications of crop plants for the purposes of modifying the starch synthesized in the plants (e.g. WO 92/11376, WO 92/14827, WO 91/19806),
transgenic crop plants which exhibit resistance to certain herbicides of the glufosinate type (e.g. EP-A-0 242 236, EP-A-0 242 246), glyphosate type (WO 92/00377) or of the sulfonylurea type (EP-A-0257993, U.S. Pat. No. 5,013,659),
transgenic crop plants, for example cotton, with the ability to produce Bacillus thuringiensis toxins (Bt toxins), which make the plants resistant to certain pests (EP-A-0 142 924, EP-A-0 193 259),
transgenic crop plants with a modified fatty acid composition (WO 91/13972),

A large number of techniques in molecular biology, with the aid of which novel transgenic plants with modified properties can be generated, are known in principle; see, for example, Sambrook et al., 1989, Molecular Cloning, A Laboratory Manual, 2nd Ed., Cold Spring Harbor Laboratory Press, Cold Spring Harbor, N.Y.; or Winnacker “Gene und Klone” [Genes and Clones], VCH Weinheim 2nd Edition 1996 or Christou, “Trends in Plant Science” 1 (1996) 423431. To carry out such recombinant manipulations, nucleic acid molecules can be introduced into plasmids which permit a mutagenesis or a sequence alteration by recombination of DNA sequences. With the aid of the abovementioned standard processes, it is possible, for example, to carry out base substitutions, to remove part sequences or to add natural or synthetic sequences. The fragments can be provided with adapters or linkers to link the DNA fragments to each other.

Plant cells with a reduced activity of a gene product can be obtained, for example, by expressing at least one corresponding antisense RNA, a sense RNA for achieving a cosuppression effect, or the expression of at least one suitably constructed ribozyme which specifically cleaves transcripts of the abovementioned gene product.

To this end, it is possible, on the one hand, to use DNA molecules which encompass all of the coding sequence of a gene product including any flanking sequences which may be present, but also DNA molecules which only encompass portions of the coding sequence, it being necessary for these portions to be so long as to cause an antisense effect in the cells. Another possibility is the use of DNA sequences which have a high degree of homology with the coding sequences of a gene product, but are not completely identical.

When expressing nucleic acid molecules in plants, the protein synthesized may be localized in any desired compartment of the plant cell. However, to achieve localization in a particular compartment, the coding region can, for example, be linked to DNA sequences which ensure localization in a particular compartment. Such sequences are known to the skilled worker (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106).

The transgenic plant cells can be regenerated by known techniques to give intact plants. In principle, the transgenic plants can be plants of any desired plant species, i.e. both monocotyledonous and dicotyledonous plants. Thus, transgenic plants can be obtained which exhibit modified properties owing to the overexpression, suppression or inhibition of homologous (i.e. natural) genes or gene sequences or expression of heterologous (i.e. foreign) genes or gene sequences.

When using the active substances according to the invention in transgenic crops, effects are frequently observed—in addition to the effects against weed plants to be observed in other crops—which are specific for the application in the transgenic crop in question, for example a modified or specifically widened controllable weed spectrum, modified application rates which may be employed for the application, preferably good combining ability with the herbicides to which the transgenic crop is resistant, and an effect on the growth and yield of the transgenic crop plants. The invention therefore also relates to the use of the compounds according to the invention as herbicides for controlling harmful plants in transgenic crop plants.

The substances according to the invention additionally have outstanding growth-regulatory properties in crop plants. They engage in the plants' metabolism in a regulatory fashion and can thus be employed for the targeted influencing of plant constituents and for facilitating harvesting, such as, for example, by triggering desiccation and stunted growth. Moreover, they are also suitable for generally controlling and inhibiting unwanted vegetative growth without destroying the plants in the process. Inhibiting the vegetative growth plays an important role in many monocotyledonous and dicotyledonous crops, allowing lodging to be reduced or prevented completely.

The compounds according to the invention can be employed in the form of wettable powders, emulsifiable concentrates, sprayable solutions, dusts or granules in the customary preparations. The invention therefore further relates also to herbicidal compositions comprising compounds of the formula (I). The compounds of the formula (I) can be formulated in various ways, depending on the prevailing biological and/or chemico-physical parameters. Examples of suitable formulations which are possible are: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), oil- or water-based dispersions, oil-miscible solutions, dusts (DP), capsule suspensions (CS), seed-dressing products, granules for spreading and soil application, granules (GR) in the form of microgranules, spray granules, coated granules and adsorption granules, water-dispersible granules (WG), water-soluble granules (SG), ULV formulations, microcapsules and waxes. These individual formulation types are known in principle and are described, for example, in Winnacker-Kuchler, “Chemische Technologie” [Chemical Technology], Volume 7, C. Hauser Verlag Munich, 4th Ed. 1986, Wade van Valkenburg, “Pesticide Formulations”, Marcel Dekker, N.Y., 1973; K. Martens, “Spray Drying” Handbook, 3rd Ed. 1979, G. Goodwin Ltd. London.

The formulation auxiliaries required, such as inert materials, surfactants, solvents and further additives, are likewise known and are described, for example, in: Watkins, “Handbook of Insecticide Dust Diluents and Carriers”, 2nd Ed., Darland Books, Caldwell N.J., H. v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers Annual”, MC Publ. Corp., Ridgewood N.J.; Sisley and Wood, “Encyclopedia of Surface Active Agents”, Chem. PubI. Co. Inc., N.Y. 1964; Schonfeldt, “Grenzflächenaktive Äthylenoxidaddukte” [Surface-active ethylene oxide adducts], Wiss. Verlagsgesell., Stuttgart 1976; Winnacker-Kuchler, “Chemische Technologie”, Volume 7, C. Hauser Verlag Munich, 4th Ed. 1986.

Wettable powders are preparations which are uniformly dispersible in water and which, in addition to the active substance, also contain ionic and/or nonionic surfactants (wetters, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium 2,2′-dinaphthylmethane-6,6′-disulfonate, sodium lignosulfonate, sodium dibutylnaphthalenesulfonate or else sodium oleoylmethyltaurate, in addition to a diluent or inert substance. To prepare the wettable powders, the herbicidal active substances are ground finely, for example in customary equipment such as hammer mills, blowing mills and air-jet mills, and simultaneously or subsequently mixed with the formulation auxiliaries.

Emulsifiable concentrates are prepared by dissolving the active substance in an organic solvent, such as butanol, cyclohexanone, dimethylformamide, xylene or else higher-boiling aromatics or hydrocarbons or mixtures of these solvents with addition of one or more ionic and/or nonionic surfactants (emulsifiers). Examples of emulsifiers which can be used are: calcium alkylarylsulfonate salts such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensates, alkyl polyethers, sorbitan esters such as, for example, sorbitan fatty acid esters or polyoxyethylene sorbitan esters such as, for example, polyoxyethylene sorbitan fatty acid esters.

Dusts are obtained by grinding the active substance with finely divided solid materials, for example talc, natural clays such as kaolin, bentonite and pyrophyllite, or diatomaceous earth. Suspension concentrates can be water based or oil based. They can be prepared for example by wet-grinding by means of customary bead mills, if appropriate with addition of surfactants, as have already been mentioned for example above in the case of the other formulation types.

Emulsions, for example oil-in-water emulsions (EW), can be prepared for example by means of stirrers, colloid mills and/or static mixers using aqueous organic solvents and, if appropriate, surfactants as have already been mentioned for example above in the case of the other formulation types.

Granules can be prepared either by spraying the active substance onto adsorptive, granulated inert material or by applying active substance concentrates to the surface of carriers such as sand, kaolinites or granulated inert material with the aid of tackifiers, for example polyvinyl alcohol, sodium polyacrylate or else mineral oils. Suitable active substances can also be granulated in the fashion which is conventional for the production of fertilizer granules, if desired as a mixture with fertilizers. Water-dispersible granules are generally prepared by customary methods such as spray drying, fluidized-bed granulation, disk granulation, mixing with high-speed stirrers and extrusion without solid inert material.

To prepare disk granules, fluidized-bed granules, extruder granules and spray granules, see, for example, processes in “Spray-Drying Handbook” 3rd ed. 1979, G. Goodwin Ltd., London; J. E. Browning, “Agglomeration”, Chemical and Engineering 1967, pages 147 et seq.; “Perry's Chemical Engineer's Handbook”, 5th Ed., McGraw-Hill, New York 1973, pp. 8-57. For further details on the formulation of crop protection products see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

As a rule, the agrochemical preparations comprise 0.1 to 99% by weight, in particular 0.1 to 95% by weight, of active substance of the formula (I). In wettable powders, the active substance concentration is, for example, approximately 10 to 90% by weight, the remainder to 100% by weight being composed of customary formulation constituents. In the case of emulsifiable concentrates, the active substance concentration can amount to approximately 1 to 90, preferably 5 to 80% by weight. Formulations in the form of dusts comprise 1 to 30% by weight of active substance, preferably in most cases 5 to 20% by weight of active substance, and sprayable solutions comprise approximately 0.05 to 80, preferably 2 to 50% by weight of active substance. In the case of water-dispersible granules, the active substance content depends partly on whether the active compound is in liquid or solid form and on the granulation auxiliaries, fillers and the like which are being used. In the case of the water-dispersible granules, for example, the active substance content is between 1 and 95% by weight, preferably between 10 and 80% by weight.

In addition, the active substance formulations mentioned comprise, if appropriate, the stickers, wetters, dispersants, emulsifiers, penetrants, preservatives, antifreeze agents, solvents, fillers, carriers, colorants, antifoams, evaporation inhibitors, and pH and viscosity regulators which are conventional in each case.

Based on these formulations, it is also possible to prepare combinations with other pesticidally active substances such as, for example, insecticides, acaricides, herbicides, fungicides; and with safeners, fertilizers and/or growth regulators, for example in the form of a readymix or a tank mix.

Active substances which can be employed in combination with the active substances according to the invention in mixed formulations or in a tank mix are, for example, known active substances as are described, for example, in Weed Research 26, 441-445 (1986) or “The Pesticide Manual”, 13th edition, The British Crop Protection Council and the Royal Soc. of Chemistry, 2003 and literature cited therein. Known herbicides which are to be mentioned, and can be combined with the compounds of the formula (I), are, for example, the following active substances (note: the compounds are either designated by the common name according to the International Organization for Standardization (ISO) or using the chemical name, if appropriate together with a customary code number):

acetochlor; acifluorfen; aclonifen; AKH 7088, i.e. [[[1-[5-[2-chloro-4-(trifluoromethyl)-phenoxy]-2-nitrophenyl]-2-methoxyethylidene]amino]oxy]acetic acid and its methyl ester; alachlor; alloxydim; ametryn; amidosulfuron; amitrol; AMS, i.e. ammonium sulfamate; anilofos; asulam; atrazine; azimsulfurone (DPX-A8947); aziprotryn; barban; BAS 516H, i.e. 5-fluoro-2-phenyl-4H-3,1-benzoxazin-4-one; benazolin; benfluralin; benfuresate; bensulfuron-methyl; bensulide; bentazone; benzofenap; benzofluor; benzoylprop-ethyl; benzthiazuron; bialaphos; bifenox; bromacil; bromobutide; bromofenoxim; bromoxynil; bromuron; buminafos; busoxinone; butachlor; butamifos; butenachlor; buthidazole; butralin; butylate; cafenstrole (CH-900); carbetamide; cafentrazone (ICI-A0051); CDAA, i.e. 2-chloro-N,N-di-2-propenylacetamide; CDEC, i.e. 2-chloroallyl diethyldithiocarbamate; chlomethoxyfen; chloramben; chlorazifop-butyl, chlormesulon (ICI-A0051); chlorbromuron; chlorbufam; chlorfenac; chlorflurecol-methyl; chloridazon; chlorimuron ethyl; chlornitrofen; chlorotoluron; chloroxuron; chlorpropham; chlorsulfuron; chlorthal-dimethyl; chlorthiamid; cinmethylin; cinosulfuron; clethodim; clodinafop and its ester derivatives (for example clodinafop-propargyl); clomazone; clomeprop; cloproxydim; clopyralid; cumyluron (JC 940); cyanazine; cycloate; cyclosulfamuron (AC 104); cycloxydim; cycluron; cyhalofop and its ester derivatives (for example butyl ester, DEH-112); cyperquat; cyprazine; cyprazole; daimuron; 2,4-DB; dalapon; desmedipham; desmetryn, di-allate; dicamba; dichlobenil; dichlorprop; diclofop and its esters such as diclofop-methyl; diethatyl; difenoxuron; difenzoquat; diflufenican; dimefuron; dimethachlor; dimethametryn; dimethenamid (SAN-582H); dimethazone, clomazon; dimethipin; dimetrasulfuron, dinitramine; dinoseb; dinoterb; diphenamid; dipropetryn; diquat; dithiopyr; diuron; DNOC; eglinazine-ethyl; EL 77, i.e. 5-cyano-1-(1,1-dimethylethyl)-N-methyl-1H-pyrazole-4-carboxamide; endothal; EPTC; esprocarb; ethalfluralin; ethametsulfuron-methyl; ethidimuron; ethiozin; ethofumesate; F5231, i.e. N-[2-chloro-4-fluoro-5-[4-(3-fluoropropyl)-4,5-dihydro-5-oxo-1H-tetrazol-1-yl]phenyl]ethanesulfonamide; ethoxyfen and its esters (for example ethyl ester, HN-252); etobenzanid (HW 52); fenoprop; fenoxan, fenoxaprop and fenoxaprop-P and their esters, for example fenoxaprop-P-ethyl and fenoxaprop-ethyl; fenoxydim; fenuron; flamprop-methyl; flazasulfuron; fluazifop and fluazifop-P and their esters, for example fluazifop-butyl and fluazifop-P-butyl; fluchloralin; flumetsulam; flumeturon; flumiclorac and its esters (for example pentyl ester, S-23031); flumioxazin (S482); flumipropyn; flupoxam (KNW-739); fluorodifen; fluoroglycofen-ethyl; flupropacil (UBIC-4243); fluridone; flurochloridone; fluroxypyr; flurtamone; fomesafen; fosamine; furyloxyfen; glufosinate; glyphosate; halosafen; halosulfuron and its esters (for example methyl ester, NC-319); haloxyfop and its esters; haloxyfop-P (═R-haloxyfop) and its esters; hexazinone; imazapyr; imazamethabenz-methyl; imazaquin and salts such as the ammonium salt; ioxynil; imazethamethapyr; imazethapyr; imazosulfuron; isocarbamid; isopropalin; isoproturon; isouron; isoxaben; isoxapyrifop; karbutilate; lactofen; lenacil; linuron; MCPA; MCPB; mecoprop; mefenacet; mefluidid; metamitron; metazachlor; metham; methabenzthiazuron; methazole; methoxyphenone; methyldymron; metabenzuron, methobenzuron; metobromuron; metolachlor; metosulam (XRD 511); metoxuron; metribuzin; metsulfuron-methyl; MH; molinate; monalide; monolinuron; monuron; monocarbamide dihydrogensulfate; MT 128, i.e. 6-chloro-N-(3-chloro-2-propenyl)-5-methyl-N-phenyl-3-pyridazinamine; MT 5950, i.e. N-[3-chloro-4-(1-methylethyl)phenyl]-2-methylpentanamide; naproanilide; napropamide; naptalam; NC 310, i.e. 4-(2,4-dichlorobenzoyl)-1-methyl-5-benzyloxypyrazole; neburon; nicosulfuron; nipyraclophen; nitralin; nitrofen; nitrofluorfen; norflurazon; orbencarb; oryzalin; oxadiargyl (RP-020630); oxadiazon; oxyfluorfen; paraquat; pebulate; pendimethalin; perfluidone; phenisopham; phenmedipham; picloram; pinoxaden; piperophos; piributicarb; pirifenop-butyl; pretilachlor; primisulfuron-methyl; procyazine; prodiamine; profluralin; proglinazine-ethyl; prometon; prometryn; propachlor; propanil; propaquizafop and its esters; propazine; propham; propisochlor; propyzamide; prosulfalin; prosulfocarb; prosulfuron (CGA-152005); prynachlor; pyraclonil; pyrazolinate; pyrazon; pyrazosulfuron-ethyl; pyrazoxyfen; pyridate; pyrithiobac (KIH-2031); pyroxofop and its esters (for example propargyl ester); quinclorac; quinmerac; quinofop and its ester derivatives, quizalofop and quizalofop-P and their ester derivatives for example quizalofop-ethyl; quizalofop-P-tefuryl and -ethyl; renriduron; rimsulfuron (DPX-E 9636); S 275, i.e. 2-[4-chloro-2-fluoro-5-(2-propynyloxy)phenyl]4, 5,6,7-tetrahydro-2H-indazole; secbumeton; sethoxydim; siduron; simazine; simetryn; SN 106279, i.e. 2-[[7-[2-chloro-4-(trifluoromethyl)phenoxy]-2-naphthalenyl]oxy]propanoic acid and its methyl ester; sulfentrazon (FMC-97285, F-6285); sulfazuron; sulfometuron-methyl; sulfosate (ICI-A0224); TCA; tebutam (GCP-5544); tebuthiuron; terbacil; terbucarb; terbuchlor; terbumeton; terbuthylazine; terbutryn; TFH 450, i.e. N,N-diethyl-3-[(2-ethyl-6-methylphenyl)sulfonyl]-1H-1,2,4-triazole-1-carboxamide; thenylchlor (NSK-850); thiazafluron; thiazopyr (Mon-13200); thidiazimin (SN-24085); thiobencarb; thifensulfuron-methyl; tiocarbazil; tralkoxydim; tri-allate; triasulfuron; triazofenamide; tribenuron-methyl; triclopyr; tridiphane; trietazine; trifluralin; triflusulfuron and esters (for example methyl ester, DPX-66037); trimeturon; tsitodef; vernolate; WL 110547, i.e. 5-phenoxy-1-[3-(trifluoromethyl)phenyl]-1H-tetrazole; UBH-509; D489; LS 82-556; KPP-300; NC-324; NC-330; KH-218; DPX-N8189; SC-0774; DOWCO-535; DK-8910; V-53482; PP-600; MBH-001; K1H-9201; ET-751; K1H-6127; K1H-2023 and KIH-485.

For use, the formulations, which are present in commercially available form, are, if appropriate, diluted in the customary manner, for example using water in the case of wettable powders, emulsifiable concentrates, dispersions and water-dispersible granules. Preparations in the form of dusts, soil granules, granules for spreading and sprayable solutions are usually not diluted any further with other inert substances prior to use. The required application rate of the compounds of the formula (I) varies with the external conditions such as, inter alia, temperature, humidity and the nature of the herbicide used. It can vary within wide limits, for example between 0.001 and 1.0 kg/ha or more of active substance, but it is preferably between 5 and 750 g/ha, in particular between 5 and 250 g/ha.

The examples which follow illustrate the invention.

### A. CHEMICAL EXAMPLES

### 1. Preparation of N-[(4-ethyl-6-{[2-(trifluoromethyl)pyridin-4-yl]oxy}pyrimid in-2-yl)methyl]cyclopropanecarboxamide (Example No. 306 from Table 3)

A mixture of 0.23 g (1.41 mmol) of 4-hydroxy-2-trifluoromethylpyridine, 0.4 g (1.41 mmol) of N-[(4-ethyl-6-{methylsulfonyl}pyrimid in-2-yl)methyl]cyclopropane-carboxamide and 0.39 g (2.82 mmol) of K₂CO₃ in 7 ml of acetonitrile is stirred under reflux for 8 h and then allowed to stand at room temperature (RT) overnight. The mixture is poured into 20 ml of water and extracted four times with 20 ml of CH₂Cl₂. The combined organic phases are dried over Na₂SO₄ and concentrated. Chromatographic purification on silica gel (SiO₂; gradient elution: 100% of heptane→heptane/ethyl acetate (EA) 3/7; CombiFlash® Companion™; Isco, Inc.) gives 0.25 g (46%) of product.

¹H-NMR: δ [CDCl₃] 0.75 (m, 2H), 0.95 (m, 2H), 1.35 (t, 3H), 1.42 (m, 1H), 1.85 (q, 2H), 4.55 (d, 2H), 6.63 (bs, 1H), 6.80 (s,1H), 7.40 (dd, 1H), 7.60 (d, 1H), 8.75 (d, 1H).

### 2. Preparation of N-[(4-methyl-6-{(3-trifluoromethyl)phenoxy}pyrimidin-2-yl)methyl]cyclopropanecarboxamide, (Example No. 206 from Table 1)

A mixture of 0.19 g (1.2 mmol) of 3-hydroxybenzyltrifluoride, 0.31 g (1.2 mmol) of N-[(4-methyl-6-{methylsulfonyl}pyrimidin-2-yl)methyl]cyclopropanecarboxamide and 0.32 g (2.3 mmol) of K₂CO₃ in 5 ml of CH₃CN is stirred under reflux for 8 h and then allowed to stand at RT overnight. The mixture is poured into 10 ml of water and extracted four times with 10 ml of CH₂Cl₂. The combined organic phases are dried over Na₂SO₄ and concentrated. Chromatographic purification on silica gel (SiO₂; gradient elution: 100% of heptane→heptane/EA 1/9; CombiFlash® Companion™; Isco, Inc.) gives 0.1 g (24%) of product.

¹H-NMR: δ [CDCl₃] 0.85 (m, 2H), 0.95 (m, 2H), 1.20 (m, 1H), 2.50 (s, 3H), 4.52 (d, 2H), 6.60 (s, 1H), 6.75 (bs, 1H), 7.35 (m, 1H), 7.40 (m, 1H), 7.55 (m, 2H).

### 3. Preparation of N-[(5-methyl-4-{[5-(trifluoromethyl)-3-thienyl]oxy}pyrimidin-2-yl)methyl]cyclopropanecarboxamide, (Example No. 206 from Table 2)

A mixture of 0.2 g (1.23 mmol) of 3-hydroxy-5-trifluoromethylthiophene, 0.33 g (1.23 mmol) of N-{[5-methyl-4-(methylsulfonyl)pyrimidin-2-yl]methyl}cyclopropane-carboxamide and 0.34 g (2.45 mmol) of K₂CO₃ in 20 ml of acetonitrile is stirred under reflux for 8 h and then allowed to stand overnight. The mixture is then poured into 20 ml of water and extracted four times with 20 ml of CH₂Cl₂. The combined organic phases are dried over Na₂SO₄ and concentrated. Chromatographic purification on silica gel using EA gives 0.08 g (18%) of product.

¹H-NMR: δ [CDCl₃] 0.75 (m, 2H), 0.92 (m, 2H), 1.40 (m, 1H), 2.30 (s, 1H), 4.58 (d, 2H), 6.65 (bs, 1H), 7.38 (m, 1H), 7.40 (m, 1H), 8.20 (s, 1H).

### Preparation of N-{[4-ethyl-6-(methylsu lfonyl)pyrimidin-2-yl]methyl}cyclopropane-carboxamide

1.98 g (8.05 mmol) of m-chloroperbenzoic acid (77% max) are added to a solution of 0.81 g (3.22 mmol) of N-{[4-ethyl-6-(methylthio)pyrimidin-2-yl]methyl}cyclopropane-carboxamide in 15 ml of CH₂Cl₂, and the mixture is stirred at RT for 48 h. For work-up, the reaction mixture is added to 20 ml of sodium disulfite solution (10%) and extracted four times with 15 ml of CH₂Cl₂. The combined organic phases are washed three times with a saturated NaHCO₃ solution, dried over Na₂SO₄ and concentrated. This gives 0.90 g (98%) of product.

¹H-NMR: δ [CDCl₃] 0.80 (m, 2H), 1.00 (m, 1H), 1.38 (t, 3H), 1.55 (m, 1H), 2.95 (q, 2H), 3.25 (s, 3H), 4.78 (d, 2H), 6.70 (bs. 1H), 7.78 (s, 1H).

### Preparation of N-{[4-ethyl-6-(methlylthio)pyrimidin-2-yl]methyl}cyclopropane-carboxamide

A spatula tip of 4-dimethylaminopyridine and 0.56 g (5.4 mmol) of cyclopropanecarbonyl chloride are added successively to a solution of 0.90 g (4.9 mmol) of 1-[4-ethyl-6-(methylthio)pyrimidin-2-yl}methanamine in 15 ml of pyridine. The reaction mixture is then stirred at RT for 24 h. For work-up, the reaction mixture is added to 20 ml of H₂O and extracted repeatedly with CH₂Cl₂. The combined organic phases are dried over Na₂SO₄ and concentrated. Chromatographic purification on silica gel (SiO₂; gradient elution: 100% of heptane→heptane/ethyl acetate 1/9; CombiFlash® Companion™; Isco, Inc.) gives 0.58 g (47%) of product.

¹H-NMR: δ [CDCl₃] 0.78 (m, 2H), 1.02 (m, 2H), 1.28 (t, 3H), 1.55 (m, 1H), 2.57 (s, 3H), 2.70 (q, 2H), 4.60 (d, 2H), 6.90 (s, 1H), 6.95 (bs, 1H).

### Preparation of 1-[4-ethyl-6-(methylthio)pyrimidin-2-yl]methanamine

H₂S is introduced into a solution of 2.78 g (13.28 mmol) of 2-(azidomethyl)-4-ethyl-6-(methylthio)pyrimidine and 2.3 ml of H₂O in 23 ml pyridine until the solution is saturated. The reaction mixture is then allowed to stand at RT for 24 h. The reaction mixture is concentrated to dryness and the residue is taken up in 50 ml of H₂O. The aqueous solution is adjusted to pH 1 using 1 N HCl and extracted with CH₂Cl₂. The aqueous phase is then adjusted to pH 8.9 using 2N NaOH and extracted repeatedly with CH₂Cl₂. The combined organic phases are dried over Na₂SO₄ and concentrated. This gives 1.91 g (78.5%) of product.

¹H-NMR: δ [CDCl₃] 1.26 (t, 3H), 2.57 (s, 3H), 2.70 (q, 2H), 4.00 (s, 2H), 6.87 (s, 1H).

### Preparation of 2-(azidomethyl)-4-ethyl-6-(methylthio)pyrimidine

At 0° C. and with stirring, 3.27 g (21.5 mmol) of DBU are added dropwise to a solution of 3:3-g (17.9 mmol) of 2-hydroxymethyl-4-thiomethyl-6-ethylpyrimidine and 5.9 g (21.50 mmol) of diphenyl phosphoryl azide in 50 ml of toluene. The reaction mixture is then allowed to warm to RT and allowed to stand for 72 h. For work-up, the mixture is concentrated under reduced pressure, where the bath temperature must not exceed 40° C. Purification by column chromatography on silica gel using heptane/EA (1/1) gives 2.78 g (74%) of product which decomposes explosively above 100° C.

¹H-NMR: δ [CDCl₃] 1.28 (t, 3H), 2.59 (s, 3H), 2.70 (q, 2H), 4.40 (s, 2H), 6.5 (s, 1H).

### Preparation of 2-hydroxymethyl-4-thiomethyl-6-ethylpyrimidine

215 ml of a 1 M BCl₃ solution in CH₂Cl₂ are carefully added dropwise to a solution, cooled to −70° C., of 14.2 g (71.6 mmol) of 2-methoxymethyl-4-thiomethyl-6-ethyl-pyrimidine in 110 ml of CH₂Cl₂. The solution is then stirred at −70° C. for another 30 min, allowed to warm to RT over a period of 2 h and allowed to stand for 12 h. For work-up, 600 ml of H₂O are carefully added dropwise, with ice-cooling. The aqueous mixture is neutralized using saturated NaHCO₃ solution and extracted repeatedly with CH₂Cl₂. The combined organic phases are dried over Na₂SO₄ and concentrated. This gives 12.6 g (95.5%) of product.

¹H-NMR: δ [CDCl₃] 1.30 (t, 3H), 2.55 (s, 3H), 2.70 (q, 2H), 3.85 (bs, OH, 4.74 (s, 2H), 6.92 (s, 1H).

### Preparation of 2-methoxymethyl-4-thiomethyl-6-ethylpyrimidine

7.9 g (112.5 mmol) of sodium thiomethoxide are added to a solution of 15 g (80.4 mmol) of 2-methoxymethyl-4-chloro-6-ethylpyrimidine, and this reaction mixture is stirred at RT for 24 h. For work-up, the precipitated solid is filtered off with suction. Concentration of the mother liquor gives 14.2 g (89%) of product.

¹H-NMR: δ [CDCl₃] 1.28 (t, 3H), 2.58 (s, 3H), 2.73 (q, 2H), 3.55 (s, 3H), 4.60 (s, 2H), 6.92 (s, 1H).

### Preparation of 2-methoxymethyl-4-chloro-6-ethylpyrimidine

38.4 g (228 mmol) of 2-methoxymethyl-4-hydroxy-6-ethylpyrimidine are initially charged-in 200 ml of chloroform, and 105 g (684 mmol) of phosphorus oxychloride are added. The reaction mixture is stirred under reflux for 3 h. At 50° C., H₂O is then added carefully until no further evolution of gas can be observed. The aqueous mixture is adjusted to pH 6-7 using saturated NaHCO₃ solution and extracted repeatedly with CH₂Cl₂. The combined organic phases are dried over Na₂SO₄ and concentrated. Purification by column chromatography on silica gel using heptane/EA (1/1) gives 29.4 g (69%) of product.

¹H-NMR: δ [CDCl₃] 1.35 (t, 3H), 2.84 (q, 2H), 3.55 (s, 3H), 4.65 (s, 2H), 7.14 (s, 1H).

### Preparation of 2-methoxymethyl-4-hydroxy-6-ethylpyrimidine

116 ml of a 30% strength sodium methoxide solution are diluted with 100 ml of methanol and, with ice-cooling, a solution of 26 g (208.7 mmol) of methoxy-acetamidinium hydrochloride in 200 ml of methanol is added dropwise. After the dropwise addition, the mixture is stirred for 1 h, and a solution of 27.1 g (208.7 mmol) of methyl propionyl acetate in 100 ml of methanol is then added dropwise at RT. The reaction mixture is stirred at RT for 96 h. For work-up, the reaction mixture is concentrated, the residue is taken up in 100 ml of H₂O and the aqueous mixture is adjusted to pH 6 using concentrated HCl. The mixture is then concentrated and the residue is taken up in 30 ml of methanol. The solid is filtered off with suction, and concentration of the mother liquor gives 38.5 g of product.

¹H-NMR: δ [CDCl₃] 1.20 (t, 3H), 2.50 (q, 2H), 3.42 (s, 3H), 4.35 (s, 2H), 6.04 (s, 1H).

### Preparation of 2-methoxymethyl-4-hydroxy-5-ethylpyrimidine

111 ml of a 30% strength sodium methoxide solution are added to a solution of 44.7 g (261 mmol) of ethyl 2-[(dimethylamino)methylene]butanoate and 42.2 g (339 mmol) of methoxyacetamidinium hydrochloride in 680 ml of ethanol, and this reaction mixture is stirred under reflux for 8 h. The reaction mixture is then allowed to stand at RT for 72 h and subsequently concentrated under reduced pressure. The residue is dissolved in H₂O, adjusted to pH 5 using concentrated HCl and extracted repeatedly with CH₂Cl₂. The combined organic phases are dried over Na₂SO₄ and concentrated. Purification by column chromatography on silica gel using EA/ethanol (7:3) gives 37.7 g (86%) of product.

¹H-NMR: δ [CDCl₃]1.20 (t, 3H), 2.50 (q, 2H), 3.52 (s, 3H), 4.38 (s, 2H), 7.75 (s, 1H).

### 4. Preparation of 2-methoxymethyl-4-thiomethyl-6-methoxypyrimidine

82 g (0.51 mol) of diethyl malonate and 64 g (0.51 mol) of methoxymethylacetamidinium hydrochloride, dissolved in 100 ml of DMF, are carefully added successively to a mixture of 210 ml of 30% strength NaOMe solution and 220 ml of DMF. The mixture is then slowly heated to 130° C. and stirred at this temperature for 3 h. For work-up, the reaction mixture is concentrated to half of its original volume and the residue that remains is taken up in 750 ml of H₂O. This mixture is warmed to 60° C. and adjusted to pH 1 using concentrated HCl. The solution obtained in this manner is, for crystallization, placed into a fridge. The precipitated solid is filtered off with suction and dried under high vacuum. This gives 64 g (80%) of 2-methoxymethyl-4,6-dihydroxypyrimidine as a colorless solid.

¹H-NMR (DMSO): δ 3.30 (s, 3H), 4.21 (s, 2H), 5.20 (s, 1H), 11.75 (bs, 2H).

A mixture of 25 g (0.16 mol) of 2-methoxymethyl-4,6-dihydroxypyrimidine, 370 g (2.4 mol) of POCl₃ and 66 ml of acetonitrile is stirred under reflux for a number of hours. For work-up, the reaction mixture is concentrated to dryness, and H₂O is carefully added to the residue that remains. The aqueous phase is extracted with CH₂Cl₂. The combined organic phases are dried over Na₂SO₄ and then concentrated. The crude product obtained in this manner is purified by column chromatography on silica gel using heptane/ethyl acetate (7/3) as mobile phase. This gives 25 g (83%) of 2-methoxymethyl4,6-dichloropyrimidine as a colorless solid; m.p. 51° C.

¹H-NMR (CDCl₃): δ 3.55 (s, 3H), 4.65 (s, 2H), 7.38 (s, 1H).

24.5 ml of a 30% strength sodium methoxide solution are added to a solution, cooled to 0° C., of 21.3 g (0.11 mol) of 2-methoxymethyl-4,6-dichloropyrimidine in 120 ml of THF, and this mixture is stirred at 0° C. for 1 h. Aqueous work-up and extraction with CH₂Cl₂ gives, after concentration of the organic phase, 20.6 g (98%) of 2-methoxy-methyl-4-methoxy-6-chloropyrimidine as an oil which is sufficiently pure for the subsequent reaction (2-methoxymethyl4,6-dimethoxypyrimidine was identified as a byproduct).

¹H-NMR (CDCl₃): δ 3.32 (s, 3H), 3.80 (s, 3H), 4.35 (s, 2H), 6.43 (s, 1H).

13.3 g (0.19 mmol) of sodium thiomethoxide are added to a solution of 23.8 g (0.126 mol) of 2-methoxymethyl-4-methoxy-6-chloropyrimidine in 400 ml of THF, and this mixture is stirred at room temperature for 16 h. The precipitated solid is filtered off with suction, and the mother liquor is concentrated to dryness. The crude product obtained in this manner is purified by silica gel column chromatography using heptane/ethyl acetate (7/3) as mobile phase. This gives 22.2 g (82%) of 2-methoxy-methyl-4-thiomethyl-6-methoxypyrimidine as an oil.

¹H-NMR (CDCl₃): δ 3.55 (s, 3H), 3.98 (s, 3H), 4.55 (s, 2H), 6.41 (s, 1H).

The examples listed in Tables 1 to 6 below were prepared analogously to the above methods or are obtainable analogously to the above methods.

The abbreviations used have the following meanings:

| | | | |
|---|---|---|---|
| Bu = n-butyl | i-Bu = isobutyl | c-Bu = cyclobutyl | t-Bu = tert-butyl |
| Pr = n-propyl | i-Pr = isopropyl | c-Pr = | Ph = phenyl |
| | | cyclopropyl | |
| Et = ethyl | Me = methyl | c = cyclo | |

TABLE 1
Compounds of the formula (I) according to the invention in which A is
A1 and X¹, X² are each hydrogen

| | |
|---|---|
| | (I) |
| 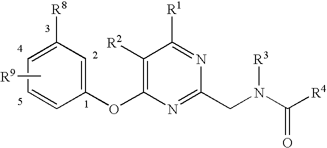 | |

| No. | R¹ | R² | R³ | R⁴ | R⁸ | R⁹ | ¹H-NMR: δ[CDCl₃] |
|---|---|---|---|---|---|---|---|
| 1 | H | H | H | H | H | 3-NO₂ | |
| 2 | H | H | H | Me | CF₃ | H | |
| 3 | H | H | H | Et | CN | H | |
| 4 | H | H | H | Pr | CF₃ | H | |
| 5 | H | H | H | i-Pr | CF₃ | 5-CF₃ | |
| 6 | H | H | H | c-Pr | Cl | 5-Cl | |
| 7 | H | H | H | Bu | CF₃ | H | |
| 8 | H | H | H | i-Bu | H | 3-NO₂ | |
| 9 | H | H | H | c-Bu | CF₃ | H | |
| 10 | H | H | H | t-Bu | ON | H | |
| 11 | H | H | H | c-hexyl | CF₃ | H | |
| 12 | H | H | H | CH₂CH═CH₂ | CF₃ | 5-CF₃ | |
| 13 | H | H | H | CH═CHCH₃ | Cl | 5-Cl | |
| 14 | H | H | H | CH═CH₂ | CF₃ | H | |
| 15 | H | H | H | CH₂C≡CH | H | 3-NO₂ | |
| 16 | H | H | H | C≡CCH₃ | CF₃ | H | |
| 17 | H | H | H | CH₂-c-Pr | CN | H | |
| 18 | H | H | H | CH₂-c-hexyl | CF₃ | H | |
| 19 | H | H | H | CF₃ | CF₃ | 5-CF₃ | |
| 20 | H | H | H | CHFCH₂CH₃ | Cl | 5-Cl | |
| 21 | H | H | H | CHClCH₃ | CF₃ | H | |
| 22 | H | H | H | CH₂OCH₃ | H | 3-NO₂ | |
| 23 | H | H | H | CH₂OCH₂CH₃ | CF₃ | H | |
| 24 | H | H | H | CF(CH₃)₂ | CN | H | |
| 25 | H | H | H | 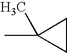 | CF₃ | H | |
| 26 | H | H | Me | H | CF₃ | 5-CF₃ | |
| 27 | H | H | Me | Me | Cl | 5-Cl | |
| 28 | H | H | Me | Et | CF₃ | H | |
| 29 | H | H | Me | Pr | H | 3-NO₂ | |
| 30 | H | H | Me | i-Pr | CF₃ | H | |
| 31 | H | H | Me | c-Pr | CN | H | |
| 32 | H | H | Me | Bu | CF₃ | H | |
| 33 | H | H | Me | i-Bu | CF₃ | 5-CF₃ | |
| 34 | H | H | Me | c-Bu | Cl | 5-Cl | |
| 35 | H | H | Me | t-Bu | CF₃ | H | |
| 36 | H | H | Me | c-hexyl | H | 3-NO₂ | |
| 37 | H | H | Me | CH₂CHCH₂ | CF₃ | H | |
| 38 | H | H | Me | CH═CHCH₃ | CN | H | |
| 39 | H | H | Me | CH═CH₂ | CF₃ | H | |
| 40 | H | H | Me | CH₂C≡CH | CF₃ | 5-CF₃ | |
| 41 | H | H | Me | C≡CCH₃ | Cl | 5-Cl | |
| 42 | H | H | Me | CH₂-c-Pr | CF₃ | H | |
| 43 | H | H | Me | CH₂-c-hexyl | H | 3-NO₂ | |
| 44 | H | H | Me | CF₃ | CF₃ | H | |
| 45 | H | H | Me | CHFCH₂CH₃ | CN | H | |
| 46 | H | H | Me | CHClCH₃ | CF₃ | H | |
| 47 | H | H | Me | CH₂OCH₃ | CF₃ | 5-CF₃ | |
| 48 | H | H | Me | CH₂OCH₂CH₃ | Cl | 5-Cl | |
| 49 | H | H | Me | CF(CH₃)₂ | CF₃ | H | |
| 50 | H | H | Me | 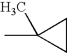 | H | 3-NO₂ | |
| 51 | H | H | CO—Me | H | CF₃ | H | |
| 52 | H | H | CO—Me | Me | CN | H | |
| 53 | H | H | CO—Me | Et | CF₃ | H | |
| 54 | H | H | CO—Me | Pr | CF₃ | 5-CF₃ | |
| 55 | H | H | CO—Me | i-Pr | Cl | 5-Cl | |
| 56 | H | H | CO—Me | c-Pr | CF₃ | H | |
| 57 | H | H | CO—Me | Bu | H | 3-NO₂ | |
| 58 | H | H | CO—Me | i-Bu | CF₃ | H | |
| 59 | H | H | CO—Me | c-Bu | CN | H | |
| 60 | H | H | CO—Me | t-Bu | CF₃ | H | |
| 61 | H | H | CO—Me | c-hexyl | CF₃ | 5-CF₃ | |
| 62 | H | H | CO—Me | CH₂CH═CH₂ | Cl | 5-Cl | |
| 63 | H | H | CO—Me | CH═CHCH₃ | CF₃ | H | |
| 64 | H | H | CO—Me | CH═CH₂ | H | 3-NO₂ | |
| 65 | H | H | CO—Me | CH₂C≡CH | CF₃ | H | |
| 66 | H | H | CO—Me | C≡CCH₃ | CN | H | |
| 67 | H | H | CO—Me | CH₂-c-Pr | CF₃ | H | |
| 68 | H | H | CO—Me | CH₂-c-hexyl | CF₃ | 5-CF₃ | |
| 69 | H | H | CO—Me | CF₃ | Cl | 5-Cl | |
| 70 | H | H | CO—Me | CHFCH₂CH₃ | CF₃ | H | |
| 71 | H | H | CO—Me | CHClCH₃ | H | 3-NO₂ | |
| 72 | H | H | CO—Me | CH₂OCH₃ | CF₃ | H | |
| 73 | H | H | CO—Me | CH₂OCH₂CH₃ | CN | H | |
| 74 | H | H | CO—Me | CF(CH₃)₂ | CF₃ | H | |
| 75 | H | H | CO—Me | 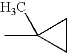 | CF₃ | 5-CF₃ | |
| 76 | H | H | CO-c-Pr | H | Cl | 5-Cl | |
| 77 | H | H | CO-c-Pr | Me | CF₃ | H | |
| 78 | H | H | CO-c-Pr | Et | H | 3-NO₂ | |
| 79 | H | H | CO-c-Pr | Pr | CF₃ | H | |
| 80 | H | H | CO-c-Pr | i-Pr | CN | H | |
| 81 | H | H | CO-c-Pr | c-Pr | CF₃ | H | |
| 82 | H | H | CO-c-Pr | Bu | CF₃ | 5-CF₃ | |
| 83 | H | H | CO-c-Pr | i-Bu | Cl | 5-Cl | |
| 84 | H | H | CO-c-Pr | c-Bu | CF₃ | H | |
| 85 | H | H | CO-c-Pr | t-Bu | H | 3-NO₂ | |
| 86 | H | H | CO-c-Pr | c-hexyl | CF₃ | H | |
| 87 | H | H | CO-c-Pr | CH₂CHCH₂ | CN | H | |
| 88 | H | H | CO-c-Pr | CH═CHCH₃ | CF₃ | H | |
| 89 | H | H | CO-c-Pr | CH═CH₂ | CF₃ | 5-CF₃ | |
| 90 | H | H | CO-c-Pr | CH₂C≡CH | Cl | 5-Cl | |
| 91 | H | H | CO-c-Pr | C≡CCH₃ | CF₃ | H | |
| 92 | H | H | CO-c-Pr | CH₂-c-Pr | H | 3-NO₂ | |
| 93 | H | H | CO-c-Pr | CH₂-c-hexyl | CF₃ | H | |
| 94 | H | H | CO-c-Pr | CF₃ | CN | H | |
| 95 | H | H | CO-c-Pr | CHFCH₂CH₃ | CF₃ | H | |
| 96 | H | H | CO-c-Pr | CHClCH₃ | CF₃ | 5-CF₃ | |
| 97 | H | H | CO-c-Pr | CH₂OCH₃ | Cl | 5-Cl | |
| 98 | H | H | CO-c-Pr | CH₂OCH₂CH₃ | CF₃ | H | |
| 99 | H | H | CO-c-Pr | CF(CH₃)₂ | H | 3-NO₂ | |
| 100 | H | H | CO-c-Pr | 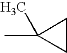 | CF₃ | H | |
| 101 | Cl | H | H | H | CN | H | |
| 102 | Cl | H | H | Me | CF₃ | H | |
| 103 | Cl | H | H | Et | CF₃ | 5-CF₃ | |
| 104 | Cl | H | H | Pr | Cl | 5-Cl | |
| 105 | Cl | H | H | i-Pr | CF₃ | H | |
| 106 | Cl | H | H | c-Pr | H | 3-NO₂ | |
| 107 | Cl | H | H | Bu | CF₃ | H | |
| 108 | Cl | H | H | i-Bu | CN | H | |
| 109 | Cl | H | H | c-Bu | CF₃ | H | |
| 110 | Cl | H | H | t-Bu | CF₃ | 5-CF₃ | |
| 111 | Cl | H | H | c-hexyl | Cl | 5-Cl | |
| 112 | Cl | H | H | CH₂CH═CH₂ | CF₃ | H | |
| 113 | Cl | H | H | CH═CHCH₃ | H | 3-NO₂ | |
| 114 | Cl | H | H | CH═CH₂ | CF₃ | H | |
| 115 | Cl | H | H | CH₂C≡CH | CN | H | |
| 116 | Cl | H | H | C≡CCH₃ | CF₃ | H | |
| 117 | Cl | H | H | CH₂-c-Pr | CF₃ | 5-CF₃ | |
| 118 | Cl | H | H | CH₂-c-hexyl | Cl | 5-Cl | |
| 119 | Cl | H | H | CF₃ | CF₃ | H | |
| 120 | Cl | H | H | CHFCH₂CH₃ | H | 3-NO₂ | |
| 121 | Cl | H | H | CHClCH₃ | CF₃ | H | |
| 122 | Cl | H | H | CH₂OCH₃ | CN | H | |
| 123 | Cl | H | H | CH₂OCH₂CH₃ | CF₃ | H | |
| 124 | Cl | H | H | CF(CH₃)₂ | CF₃ | 5-CF₃ | |
| 125 | Cl | H | H | 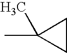 | Cl | 5-Cl | |
| 126 | Cl | H | Me | H | CF₃ | H | |
| 127 | Cl | H | Me | Me | H | 3-NO₂ | |
| 128 | Cl | H | Me | Et | CF₃ | H | |
| 129 | Cl | H | Me | Pr | CN | H | |
| 130 | Cl | H | Me | i-Pr | CF₃ | H | |
| 131 | Cl | H | Me | c-Pr | CF₃ | 5-CF₃ | |
| 132 | Cl | H | Me | Bu | Cl | 5-Cl | |
| 133 | Cl | H | Me | i-Bu | CF₃ | H | |
| 134 | Cl | H | Me | c-Bu | H | 3-NO₂ | |
| 135 | Cl | H | Me | t-Bu | CF₃ | H | |
| 136 | Cl | H | Me | c-hexyl | CN | H | |
| 137 | Cl | H | Me | CH₂CH═CH₂ | CF₃ | H | |
| 138 | Cl | H | Me | CH═CHCH₃ | CF₃ | 5-CF₃ | |
| 139 | Cl | H | Me | CH═CH₂ | Cl | 5-Cl | |
| 140 | Cl | H | Me | CH₂C≡CH | CF₃ | H | |
| 141 | Cl | H | Me | C≡CCH₃ | H | 3-NO₂ | |
| 142 | Cl | H | Me | CH₂-c-Pr | CF₃ | H | |
| 143 | Cl | H | Me | CH₂-c-hexyl | CN | H | |
| 144 | Cl | H | Me | CF₃ | CF₃ | H | |
| 145 | Cl | H | Me | CHFCH₂CH₃ | CF₃ | 5-CF₃ | |
| 146 | Cl | H | Me | CHClCH₃ | Cl | 5-Cl | |
| 147 | Cl | H | Me | CH₂OCH₃ | CF₃ | H | |
| 148 | Cl | H | Me | CH₂OCH₂CH₃ | H | 3-NO₂ | |
| 149 | Cl | H | Me | CF(CH₃)₂ | CF₃ | H | |
| 150 | Cl | H | Me | 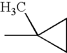 | CN | H | |
| 151 | Cl | H | CO—Me | H | CF₃ | H | |
| 152 | Cl | H | CO—Me | Me | CF₃ | 5-CF₃ | |
| 153 | Cl | H | CO—Me | Et | Cl | 5-Cl | |
| 154 | Cl | H | CO—Me | Pr | CF₃ | H | |
| 155 | Cl | H | CO—Me | i-Pr | H | 3-NO₂ | |
| 156 | Cl | H | CO—Me | c-Pr | CF₃ | H | |
| 157 | Cl | H | CO—Me | Bu | CN | H | |
| 158 | Cl | H | CO—Me | i-Bu | CF₃ | H | |
| 159 | Cl | H | CO—Me | c-Bu | CF₃ | 5-CF₃ | |
| 160 | Cl | H | CO—Me | t-Bu | Cl | 5-Cl | |
| 161 | Cl | H | CO—Me | c-hexyl | CF₃ | H | |
| 162 | Cl | H | CO—Me | CH₂CH═CH₂ | H | 3-NO₂ | |
| 163 | Cl | H | CO—Me | CH═CHCH₃ | CF₃ | H | |
| 164 | Cl | H | CO—Me | CH═CH₂ | CN | H | |
| 165 | Cl | H | CO—Me | CH₂C≡CH | CF₃ | H | |
| 166 | Cl | H | CO—Me | C≡CCH₃ | CF₃ | 5-CF₃ | |
| 167 | Cl | H | CO—Me | CH₂-c-Pr | Cl | 5-Cl | |
| 168 | Cl | H | CO—Me | CH₂-c-hexyl | CF₃ | H | |
| 169 | Cl | H | CO—Me | CF₃ | H | 3-NO₂ | |
| 170 | Cl | H | CO—Me | CHFCH₂CH₃ | CF₃ | H | |
| 171 | Cl | H | CO—Me | CHClCH₃ | CN | H | |
| 172 | Cl | H | CO—Me | CH₂OCH₃ | CF₃ | H | |
| 173 | Cl | H | CO—Me | CH₂OCH₂CH₃ | CF₃ | 5-CF₃ | |
| 174 | Cl | H | CO—Me | CF(CH₃)₂ | Cl | 5-Cl | |
| 175 | Cl | H | CO—Me | 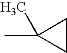 | CF₃ | H | |
| 176 | Cl | H | CO-c-Pr | H | H | 3-NO₂ | |
| 177 | Cl | H | CO-c-Pr | Me | CF₃ | H | |
| 178 | Cl | H | CO-c-Pr | Et | CN | H | |
| 179 | Cl | H | CO-c-Pr | Pr | CF₃ | H | |
| 180 | Cl | H | CO-c-Pr | i-Pr | CF₃ | 5-CF₃ | |
| 181 | Cl | H | CO-c-Pr | c-Pr | Cl | 5-Cl | |
| 182 | Cl | H | CO-c-Pr | Bu | CF₃ | H | |
| 183 | Cl | H | CO-c-Pr | i-Bu | H | 3-NO₂ | |
| 184 | Cl | H | CO-c-Pr | c-Bu | CF₃ | H | |
| 185 | Cl | H | CO-c-Pr | t-Bu | CN | H | |
| 186 | Cl | H | CO-c-Pr | c-hexyl | CF₃ | H | |
| 187 | Cl | H | CO-c-Pr | CH₂CHCH₂ | CF₃ | 5-CF₃ | |
| 188 | Cl | H | CO-c-Pr | CHCHCH₃ | Cl | 5-Cl | |
| 189 | Cl | H | CO-c-Pr | CH═CH₂ | CF₃ | H | |
| 190 | Cl | H | CO-c-Pr | CH₂C≡CH | H | 3-NO₂ | |
| 191 | Cl | H | CO-c-Pr | C≡CCH₃ | CF₃ | H | |
| 192 | Cl | H | CO-c-Pr | CH₂-c-Pr | CN | H | |
| 193 | Cl | H | CO-c-Pr | CH₂-c-hexyl | CF₃ | H | |
| 194 | Cl | H | CO-c-Pr | CF₃ | CF₃ | 5-CF₃ | |
| 195 | Cl | H | CO-c-Pr | CHFCH₂CH₃ | Cl | 5-Cl | |
| 196 | Cl | H | CO-c-Pr | CHClCH₃ | CF₃ | H | |
| 197 | Cl | H | CO-c-Pr | CH₂OCH₃ | H | 3-NO₂ | |
| 198 | Cl | H | CO-c-Pr | CH₂OCH₂CH₃ | CF₃ | H | |
| 199 | Cl | H | CO-c-Pr | CF(CH₃)₂ | CN | H | |
| 200 | Cl | H | CO-c-Pr | 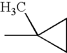 | CF₃ | H | |
| 201 | Me | H | H | H | CF₃ | 5-CF₃ | |
| 202 | Me | H | H | i-Pr | Cl | 5-Cl | 6.65 (s, 5-H, pyrimidine) |
| 203 | Me | H | H | i-Pr | CF₃ | H | 6.65 (s, 5-H, pyrimidine) |
| 204 | Me | H | H | i-Pr | H | 2,6-F₂ | |
| 205 | Me | H | H | i-Pr | H | 4-NO₂ | 6.74 (s, 5-H, pyrimidine) |
| 206 | Me | H | H | c-Pr | CF₃ | H | 0.85 (m, 2H), 0.95 (m, 2H), 1.20 |
| | | | | | | | (m, 1H), 2.50 (s, 3H), |
| | | | | | | | 4.52 (d, 2H), |
| | | | | | | | 6.60 (s, 1H), 6.75 (bs, 1H), 7.35 |
| | | | | | | | (m, 1H), 7.40 (m, 1H), 7.55 (m, |
| | | | | | | | 2H) |
| 207 | Me | H | H | i-Pr | CN | H | 6.70 (s, 5-H, pyriinidine) |
| 208 | Me | H | H | i-Bu | CF₃ | 5-CF₃ | |
| 209 | Me | H | H | c-Bu | Cl | 5-Cl | |
| 210 | Me | H | H | t-Bu | CF₃ | H | |
| 211 | Me | H | H | c-hexyl | H | 3-NO₂ | |
| 212 | Me | H | H | CH₂CHCH₂ | CF₃ | H | |
| 213 | Me | H | H | CHCHCH₃ | CN | H | |
| 214 | Me | H | H | CH═CH₂ | CF₃ | H | |
| 215 | Me | H | H | CH₂C≡CH | CF₃ | 5-CF₃ | |
| 216 | Me | H | H | C≡CCH₃ | Cl | 5-Cl | |
| 217 | Me | H | H | CH₂-c-Pr | CF₃ | H | |
| 218 | Me | H | H | CH₂-c-hexyl | H | 3-NO₂ | |
| 219 | Me | H | H | CF₃ | CF₃ | H | 6.80 (s, 5-H, pyrimidine) |
| 220 | Me | H | H | CHFCH₂CH₃ | CN | H | |
| 221 | Me | H | H | CHClCH₃ | CF₃ | H | |
| 222 | Me | H | H | CH₂OCH₃ | CF₃ | 5-CF₃ | |
| 223 | Me | H | H | CH₂OCH₂CH₃ | Cl | 5-Cl | |
| 224 | Me | H | H | CF(CH₃)₂ | CF₃ | H | |
| 225 | Me | H | H | 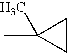 | H | 3-NO₂ | |
| 226 | Me | H | Me | H | CF₃ | H | |
| 227 | Me | H | Me | Me | CN | H | |
| 228 | Me | H | Me | Et | CF₃ | H | |
| 229 | Me | H | Me | Pr | CF₃ | 5-CF₃ | |
| 230 | Me | H | Me | i-Pr | Cl | 5-Cl | |
| 231 | Me | H | Me | c-Pr | CF₃ | H | |
| 232 | Me | H | Me | Bu | H | 3-NO₂ | |
| 233 | Me | H | Me | i-Bu | CF₃ | H | |
| 234 | Me | H | Me | c-Bu | CN | H | |
| 235 | Me | H | Me | t-Bu | CF₃ | H | |
| 236 | Me | H | Me | c-hexyl | CF₃ | 5-CF₃ | |
| 237 | Me | H | Me | CH₂CHCH₂ | Cl | 5-Cl | |
| 238 | Me | H | Me | CHCHCH₃ | CF₃ | H | |
| 239 | Me | H | Me | CH═CH₂ | H | 3-NO₂ | |
| 240 | Me | H | Me | CH₂C≡CH | CF₃ | H | |
| 241 | Me | H | Me | C≡CCH₃ | CN | H | |
| 242 | Me | H | Me | CH₂-c-Pr | CF₃ | H | |
| 243 | Me | H | Me | CH₂-c-hexyl | CF₃ | 5-CF₃ | |
| 244 | Me | H | Me | CF₃ | Cl | 5-Cl | |
| 245 | Me | H | Me | CHFCH₂CH₃ | CF₃ | H | |
| 246 | Me | H | Me | CHClCH₃ | H | 3-NO₂ | |
| 247 | Me | H | Me | CH₂OCH₃ | CF₃ | H | |
| 248 | Me | H | Me | CH₂OCH₂CH₃ | CN | H | |
| 249 | Me | H | Me | CF(CH₃)₂ | CF₃ | H | |
| 250 | Me | H | Me | 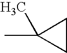 | CF₃ | 5-CF₃ | |
| 251 | Me | H | CO—Me | H | Cl | 5-Cl | |
| 252 | Me | H | CO—Me | Me | CF₃ | H | |
| 253 | Me | H | CO—Me | Et | H | 3-NO₂ | |
| 254 | Me | H | CO—Me | Pr | CF₃ | H | |
| 255 | Me | H | CO—Me | i-Pr | CN | H | |
| 256 | Me | H | CO—Me | c-Pr | CF₃ | H | |
| 257 | Me | H | CO—Me | Bu | CF₃ | 5-CF₃ | |
| 258 | Me | H | CO—Me | i-Bu | Cl | 5-Cl | |
| 259 | Me | H | CO—Me | c-Bu | CF₃ | H | |
| 260 | Me | H | CO—Me | t-Bu | H | 3-NO₂ | |
| 261 | Me | H | CO—Me | c-hexyl | CF₃ | H | |
| 262 | Me | H | CO—Me | CH₂CH═CH₂ | CN | H | |
| 263 | Me | H | CO—Me | CH═CHCH₃ | CF₃ | H | |
| 264 | Me | H | CO—Me | CH═CH₂ | CF₃ | 5-CF₃ | |
| 265 | Me | H | CO—Me | CH₂C≡CH | Cl | 5-Cl | |
| 266 | Me | H | CO—Me | C≡CCH₃ | CF₃ | H | |
| 267 | Me | H | CO—Me | CH₂-c-Pr | H | 3-NO₂ | |
| 268 | Me | H | CO—Me | CH₂-c-hexyl | CF₃ | H | |
| 269 | Me | H | CO—Me | CF₃ | CN | H | |
| 270 | Me | H | CO—Me | CHFCH₂CH₃ | CF₃ | H | |
| 271 | Me | H | CO—Me | CHClCH₃ | CF₃ | 5-CF₃ | |
| 272 | Me | H | CO—Me | CH₂OCH₃ | Cl | 5-Cl | |
| 273 | Me | H | CO—Me | CH₂OCH₂CH₃ | CF₃ | H | |
| 274 | Me | H | CO—Me | CF(CH₃)₂ | H | 3-NO₂ | |
| 275 | Me | H | CO—Me | 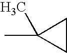 | CF₃ | H | |
| 276 | Me | H | CO-c-Pr | H | CN | H | |
| 277 | Me | H | CO-c-Pr | Me | CF₃ | H | |
| 278 | Me | H | CO-c-Pr | Et | CF₃ | 5-CF₃ | |
| 279 | Me | H | CO-c-Pr | Pr | Cl | 5-Cl | |
| 280 | Me | H | CO-c-Pr | i-Pr | CF₃ | H | |
| 281 | Me | H | CO-c-Pr | c-Pr | H | 3-NO₂ | |
| 282 | Me | H | CO-c-Pr | Bu | CF₃ | H | |
| 283 | Me | H | CO-c-Pr | i-Bu | CN | H | |
| 284 | Me | H | CO-c-Pr | c-Bu | CF₃ | H | |
| 285 | Me | H | CO-c-Pr | t-Bu | CF₃ | 5-CF₃ | |
| 286 | Me | H | CO-c-Pr | c-hexyl | Cl | 5-Cl | |
| 287 | Me | H | CO-c-Pr | CH₂CH═CH₂ | CF₃ | H | |
| 288 | Me | H | CO-c-Pr | CH═CHCH₃ | H | 3-NO₂ | |
| 289 | Me | H | CO-c-Pr | CH═CH₂ | CF₃ | H | |
| 290 | Me | H | CO-c-Pr | CH₂C≡CH | CN | H | |
| 291 | Me | H | CO-c-Pr | C≡CCH₃ | CF₃ | H | |
| 292 | Me | H | CO-c-Pr | CH₂-c-Pr | CF₃ | 5-CF₃ | |
| 293 | Me | H | CO-c-Pr | CH₂-c-hexyl | Cl | 5-Cl | |
| 294 | Me | H | CO-c-Pr | CF₃ | CF₃ | H | |
| 295 | Me | H | CO-c-Pr | CHFCH₂CH₃ | H | 3-NO₂ | |
| 296 | Me | H | CO-c-Pr | CHClCH₃ | CF₃ | H | |
| 297 | Me | H | CO-c-Pr | CH₂OCH₃ | CN | H | |
| 298 | Me | H | CO-c-Pr | CH₂OCH₂CH₃ | CF₃ | H | |
| 299 | Me | H | CO-c-Pr | CF(CH₃)₂ | CF₃ | 5-CF₃ | |
| 300 | Me | H | CO-c-Pr | 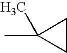 | Cl | 5-Cl | |
| 301 | Et | H | H | c-Pr | I | H | 6.60 (s, 5-H, pyrimidine) |
| 302 | Et | H | H | c-Pr | O—CF₃ | H | 6.61 (s, 5-H, pyrimidine) |
| 303 | Et | H | H | c-Pr | CN | H | 6.76 (s, 5-H, pyrimidine) |
| 304 | Et | H | H | c-Pr | CF₃ | H | 6.70 (s, 5-H, pyrimidine) |
| 305 | Et | H | H | t-Bu | CN | H | 6.66 (s, 5-H, pyrimidine) |
| 306 | Et | H | H | t-Bu | H | 4-Cl | |
| 307 | Et | H | H | t-Bu | Cl | 4-Cl | 6.64 (s, 5-H, pyrimidine) |
| 308 | Et | H | H | t-Bu | CF₃ | H | 6.60 (s, 5-H, pyrimidine) |
| 309 | Et | H | H | t-Bu | OCF₃ | H | |
| 310 | Et | H | H | t-Bu | CF₃ | H | 6.67 (s, 5-H, pyrimidine) |
| 311 | Et | H | H | c-hexyl | CN | H | |
| 312 | Et | H | H | CH₂CH═CH₂ | CF₃ | H | |
| 313 | Et | H | H | CH═CHCH₃ | CF₃ | 5-CF₃ | |
| 314 | Et | H | H | CH═CH₂ | Cl | 5-Cl | |
| 315 | Et | H | H | CH₂C≡CH | CF₃ | H | |
| 316 | Et | H | H | C≡CCH₃ | H | 3-NO₂ | |
| 317 | Et | H | H | CH₂-c-Pr | CF₃ | H | |
| 318 | Et | H | H | CH₂-c-hexyl | CN | H | |
| 319 | Et | H | H | CF₃ | CF₃ | H | 6.74 (s, 5-H, pyrimidine) |
| 320 | Et | H | H | CHFCH₂CH₃ | CF₃ | H | |
| 321 | Et | H | H | CHClCH₃ | Cl | 5-Cl | |
| 322 | Et | H | H | CH₂OCH₃ | CF₃ | H | |
| 323 | Et | H | H | CF(CH₃)₂ | CF₃ | 4-F | 6.65 (s, 5-H, pyrimidine) |
| 324 | Et | H | H | CF(CH₃)₂ | CF₃ | H | 6.68 (s, 5-H, pyrimidine) |
| 325 | Et | H | H | 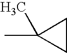 | CF₃ | H | 6.75 (s, 5-H, pyrimidine) |
| 326 | Et | H | H | 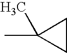 | CN | H | 6.77 (s, 5-H, pyrimidine) |
| 327 | Et | H | Me | Me | CF₃ | 5-CF₃ | |
| 328 | Et | H | Me | Et | Cl | 5-Cl | |
| 329 | Et | H | Me | Pr | CF₃ | H | |
| 330 | Et | H | Me | i-Pr | H | 3-NO₂ | |
| 331 | Et | H | Me | c-Pr | CF₃ | H | |
| 332 | Et | H | Me | Bu | CN | H | |
| 333 | Et | H | Me | i-Bu | CF₃ | H | |
| 334 | Et | H | Me | c-Bu | CF₃ | 5-CF₃ | |
| 335 | Et | H | Me | t-Bu | Cl | 5-Cl | |
| 336 | Et | H | Me | c-hexyl | CF₃ | H | |
| 337 | Et | H | Me | CH₂CHCH₂ | H | 3-NO₂ | |
| 338 | Et | H | Me | CH═CHCH₃ | CF₃ | H | |
| 339 | Et | H | Me | CH═CH₂ | CN | H | |
| 340 | Et | H | Me | CH₂C≡CH | CF₃ | H | |
| 341 | Et | H | Me | C≡CCH₃ | CF₃ | 5-CF₃ | |
| 342 | Et | H | Me | CH₂-c-Pr | Cl | 5-Cl | |
| 343 | Et | H | Me | CH₂-c-hexyl | CF₃ | H | |
| 344 | Et | H | Me | CF₃ | H | 3-NO₂ | |
| 345 | Et | H | Me | CHFCH₂CH₃ | CF₃ | H | |
| 346 | Et | H | Me | CHClCH₃ | CN | H | |
| 347 | Et | H | Me | CH₂OCH₃ | CF₃ | H | |
| 348 | Et | H | Me | CH₂OCH₂CH₃ | CF₃ | 5-CF₃ | |
| 349 | Et | H | Me | CF(CH₃)₂ | Cl | 5-Cl | |
| 350 | Et | H | Me | 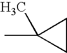 | CF₃ | H | |
| 351 | Et | H | CO—Me | H | H | 3-NO₂ | |
| 352 | Et | H | CO—Me | Me | CF₃ | H | |
| 353 | Et | H | CO—Me | Et | CN | H | |
| 354 | Et | H | CO—Me | Pr | CF₃ | H | |
| 355 | Et | H | CO—Me | i-Pr | CF₃ | 5-CF₃ | |
| 356 | Et | H | CO—Me | c-Pr | Cl | 5-Cl | |
| 357 | Et | H | CO—Me | Bu | CF₃ | H | |
| 358 | Et | H | CO—Me | i-Bu | H | 3-NO₂ | |
| 359 | Et | H | CO—Me | c-Bu | CF₃ | H | |
| 360 | Et | H | CO—Me | t-Bu | CN | H | |
| 361 | Et | H | CO—Me | c-hexyl | CF₃ | H | |
| 362 | Et | H | CO—Me | CH₂CH═CH₂ | CF₃ | 5-CF₃ | |
| 363 | Et | H | CO—Me | CH═CHCH₃ | Cl | 5-Cl | |
| 364 | Et | H | CO—Me | CHCH₂ | CF₃ | H | |
| 365 | Et | H | CO—Me | CH₂C≡CH | H | 3-NO₂ | |
| 366 | Et | H | CO—Me | C≡CCH₃ | CF₃ | H | |
| 367 | Et | H | CO—Me | CH₂-c-Pr | CN | H | |
| 368 | Et | H | CO—Me | CH₂-c-hexyl | CF₃ | H | |
| 369 | Et | H | CO—Me | CF₃ | CF₃ | 5-CF₃ | |
| 370 | Et | H | CO—Me | CHFCH₂CH₃ | Cl | 5-Cl | |
| 371 | Et | H | CO—Me | CHClCH₃ | CF₃ | H | |
| 372 | Et | H | CO—Me | CH₂OCH₃ | H | 3-NO₂ | |
| 373 | Et | H | CO—Me | CH₂OCH₂CH₃ | CF₃ | H | |
| 374 | Et | H | CO—Me | CF(CH₃)₂ | CN | H | |
| 375 | Et | H | CO—Me | 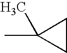 | CF₃ | H | |
| 376 | Et | H | CO-c-Pr | H | CF₃ | 5-CF₃ | |
| 377 | Et | H | CO-c-Pr | Me | Cl | 5-Cl | |
| 378 | Et | H | CO-c-Pr | Et | CF₃ | H | |
| 379 | Et | H | CO-c-Pr | Pr | H | 3-NO₂ | |
| 380 | Et | H | CO-c-Pr | i-Pr | CF₃ | H | |
| 381 | Et | H | CO-c-Pr | c-Pr | CN | H | |
| 382 | Et | H | CO-c-Pr | Bu | CF₃ | H | |
| 383 | Et | H | CO-c-Pr | i-Bu | CF₃ | 5-CF₃ | |
| 384 | Et | H | CO-c-Pr | c-Bu | Cl | 5-Cl | |
| 385 | Et | H | CO-c-Pr | t-Bu | CF₃ | H | |
| 386 | Et | H | CO-c-Pr | c-hexyl | H | 3-NO₂ | |
| 387 | Et | H | CO-c-Pr | CH₂CHCH₂ | CF₃ | H | |
| 388 | Et | H | CO-c-Pr | CH═CHCH₃ | CN | H | |
| 389 | Et | H | CO-c-Pr | CHCH₂ | CF₃ | H | |
| 390 | Et | H | CO-c-Pr | CH₂C≡CH | CF₃ | 5-CF₃ | |
| 391 | Et | H | CO-c-Pr | C≡CCH₃ | Cl | 5-Cl | |
| 392 | Et | H | CO-c-Pr | CH₂-c-Pr | CF₃ | H | |
| 393 | Et | H | CO-c-Pr | CH₂-c-hexyl | H | 3-NO₂ | |
| 394 | Et | H | CO-c-Pr | CF₃ | CF₃ | H | |
| 395 | Et | H | CO-c-Pr | CHFCH₂CH₃ | CN | H | |
| 396 | Et | H | CO-c-Pr | CHClCH₃ | CF₃ | H | |
| 397 | Et | H | CO-c-Pr | CH₂OCH₃ | CF₃ | 5-CF₃ | |
| 398 | Et | H | CO-c-Pr | CH₂OCH₂CH₃ | Cl | 5-Cl | |
| 399 | Et | H | CO-c-Pr | CF(CH₃)₂ | CF₃ | H | |
| 400 | Et | H | CO-c-Pr | 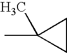 | H | 3-NO₂ | |
| 401 | OMe | H | H | H | CF₃ | H | |
| 402 | OMe | H | H | Me | CN | H | |
| 403 | OMe | H | H | Et | CF₃ | H | |
| 404 | OMe | H | H | Pr | CF₃ | 5-CF₃ | |
| 405 | OMe | H | H | i-Pr | Cl | 5-Cl | |
| 406 | OMe | H | H | c-Pr | CF₃ | H | |
| 407 | OMe | H | H | Bu | H | 3-NO₂ | |
| 408 | OMe | H | H | i-Bu | CF₃ | H | |
| 409 | OMe | H | H | c-Bu | CN | H | |
| 410 | OMe | H | H | t-Bu | CF₃ | H | |
| 411 | OMe | H | H | c-hexyl | CF₃ | 5-CF₃ | |
| 412 | OMe | H | H | CH₂CHCH₂ | Cl | 5-Cl | |
| 413 | OMe | H | H | CH═CHCH₃ | CF₃ | H | |
| 414 | OMe | H | H | CHCH₂ | H | 3-NO₂ | |
| 415 | OMe | H | H | CH₂C≡CH | CF₃ | H | |
| 416 | OMe | H | H | C≡CCH₃ | CN | H | |
| 417 | OMe | H | H | CH₂-c-Pr | CF₃ | H | |
| 418 | OMe | H | H | CH₂-c-hexyl | CF₃ | 5-CF₃ | |
| 419 | OMe | H | H | CF₃ | Cl | 5-Cl | |
| 420 | OMe | H | H | CHFCH₂CH₃ | CF₃ | H | |
| 421 | OMe | H | H | CHClCH₃ | H | 3-NO₂ | |
| 422 | OMe | H | H | CH₂OCH₃ | CF₃ | H | |
| 423 | OMe | H | H | CH₂OCH₂CH₃ | CN | H | |
| 424 | OMe | H | H | CF(CH₃)₂ | CF₃ | H | |
| 425 | OMe | H | H | 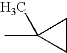 | CF₃ | 5-CF₃ | |
| 426 | OMe | H | Me | H | Cl | 5-Cl | |
| 427 | OMe | H | Me | Me | CF₃ | H | |
| 428 | OMe | H | Me | Et | H | 3-NO₂ | |
| 429 | OMe | H | Me | Pr | CF₃ | H | |
| 430 | OMe | H | Me | i-Pr | CN | H | |
| 431 | OMe | H | Me | c-Pr | CF₃ | H | |
| 432 | OMe | H | Me | Bu | CF₃ | 5-CF₃ | |
| 433 | OMe | H | Me | i-Bu | Cl | 5-Cl | |
| 434 | OMe | H | Me | c-Bu | CF₃ | H | |
| 435 | OMe | H | Me | t-Bu | H | 3-NO₂ | |
| 436 | OMe | H | Me | c-hexyl | CF₃ | H | |
| 437 | OMe | H | Me | CH₂CH═CH₂ | CN | H | |
| 438 | OMe | H | Me | CHCHCH₃ | CF₃ | H | |
| 439 | OMe | H | Me | CHCH₂ | CF₃ | 5-CF₃ | |
| 440 | OMe | H | Me | CH₂C≡CH | Cl | 5-Cl | |
| 441 | OMe | H | Me | C≡CCH₃ | CF₃ | H | |
| 442 | OMe | H | Me | CH₂-c-Pr | H | 3-NO₂ | |
| 443 | OMe | H | Me | CH₂-c-hexyl | CF₃ | H | |
| 444 | OMe | H | Me | CF₃ | CN | H | |
| 445 | OMe | H | Me | CHFCH₂CH₃ | CF₃ | H | |
| 446 | OMe | H | Me | CHClCH₃ | CF₃ | 5-CF₃ | |
| 447 | OMe | H | Me | CH₂OCH₃ | Cl | 5-Cl | |
| 448 | OMe | H | Me | CH₂OCH₂CH₃ | CF₃ | H | |
| 449 | OMe | H | Me | CF(CH₃)₂ | H | 3-NO₂ | |
| 450 | OMe | H | Me | 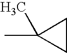 | CF₃ | H | |
| 451 | OMe | H | CO—Me | H | CN | H | |
| 452 | OMe | H | CO—Me | Me | CF₃ | H | |
| 453 | OMe | H | CO—Me | Et | CF₃ | 5-CF₃ | |
| 454 | OMe | H | CO—Me | Pr | Cl | 5-Cl | |
| 455 | OMe | H | CO—Me | i-Pr | CF₃ | H | |
| 456 | OMe | H | CO—Me | c-Pr | H | 3-NO₂ | |
| 457 | OMe | H | CO—Me | Bu | CF₃ | H | |
| 458 | OMe | H | CO—Me | i-Bu | CN | H | |
| 459 | OMe | H | CO—Me | c-Bu | CF₃ | H | |
| 460 | OMe | H | CO—Me | t-Bu | CF₃ | 5-CF₃ | |
| 461 | OMe | H | CO—Me | c-hexyl | Cl | 5-Cl | |
| 462 | OMe | H | CO—Me | CH₂CHCH₂ | CF₃ | H | |
| 463 | OMe | H | CO—Me | CHCHCH₃ | H | 3-NO₂ | |
| 464 | OMe | H | CO—Me | CHCH₂ | CF₃ | H | |
| 465 | OMe | H | CO—Me | CH₂C≡CH | CN | H | |
| 466 | OMe | H | CO—Me | C≡CCH₃ | CF₃ | H | |
| 467 | OMe | H | CO—Me | CH₂-c-Pr | CF₃ | 5-CF₃ | |
| 468 | OMe | H | CO—Me | CH₂-c-hexyl | Cl | 5-Cl | |
| 469 | OMe | H | CO—Me | CF₃ | CF₃ | H | |
| 470 | OMe | H | CO—Me | CHFCH₂CH₃ | H | 3-NO₂ | |
| 471 | OMe | H | CO—Me | CHClCH₃ | CF₃ | H | |
| 472 | OMe | H | CO—Me | CH₂OCH₃ | CN | H | |
| 473 | OMe | H | CO—Me | CH₂OCH₂CH₃ | CF₃ | H | |
| 474 | OMe | H | CO—Me | CF(CH₃)₂ | CF₃ | 5-CF₃ | |
| 475 | OMe | H | CO—Me | 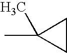 | Cl | 5-Cl | |
| 476 | OMe | H | CO-c-Pr | H | CF₃ | H | |
| 477 | OMe | H | CO-c-Pr | Me | H | 3-NO₂ | |
| 478 | OMe | H | CO-c-Pr | Et | CF₃ | H | |
| 479 | OMe | H | CO-c-Pr | Pr | CN | H | |
| 480 | OMe | H | CO-c-Pr | i-Pr | CF₃ | H | |
| 481 | OMe | H | CO-c-Pr | c-Pr | CF₃ | 5-CF₃ | |
| 482 | OMe | H | CO-c-Pr | Bu | Cl | 5-Cl | |
| 483 | OMe | H | CO-c-Pr | i-Bu | CF₃ | H | |
| 484 | OMe | H | CO-c-Pr | c-Bu | H | 3-NO₂ | |
| 485 | OMe | H | CO-c-Pr | t-Bu | CF₃ | H | |
| 486 | OMe | H | CO-c-Pr | c-hexyl | CN | H | |
| 487 | OMe | H | CO-c-Pr | CH₂CH═CH₂ | CF₃ | H | |
| 488 | OMe | H | CO-c-Pr | CH═CHCH₃ | CF₃ | 5-CF₃ | |
| 489 | OMe | H | CO-c-Pr | CH═CH₂ | Cl | 5-Cl | |
| 490 | OMe | H | CO-c-Pr | CH₂C≡CH | CF₃ | H | |
| 491 | OMe | H | CO-c-Pr | C≡CCH₃ | H | 3-NO₂ | |
| 492 | OMe | H | CO-c-Pr | CH₂-c-Pr | CF₃ | H | |
| 493 | OMe | H | CO-c-Pr | CH₂-c-hexyl | CN | H | |
| 494 | OMe | H | CO-c-Pr | CF₃ | CF₃ | H | |
| 495 | OMe | H | CO-c-Pr | CHFCH₂CH₃ | CF₃ | 5-CF₃ | |
| 496 | OMe | H | CO-c-Pr | CHClCH₃ | Cl | 5-Cl | |
| 497 | OMe | H | CO-c-Pr | CH₂OCH₃ | CF₃ | H | |
| 498 | OMe | H | CO-c-Pr | CH₂OCH₂CH₃ | H | 3-NO₂ | |
| 499 | OMe | H | CO-c-Pr | CF(CH₃)₂ | CF₃ | H | |
| 500 | OMe | H | CO-c-Pr | 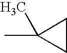 | CN | H | |
| 501 | H | Me | H | c-Pr | CF₃ | H | 8.40 (s, 6-H, pyrimidine) |
| 502 | H | Me | H | i-Pr | CF₃ | H | 8.40 (s, 6-H, pyrimidine) |
| 503 | H | Et | H | c-Pr | CF₃ | H | 8.40 (s, 6-H, pyrimidine) |
| 504 | H | Et | H | i-Pr | CF₃ | H | 8.40 (s, 6-H, pyrimidine) |
| 505 | Me | H | H | c-Bu | CF₃ | H | 6.65 (s, 5-H, pyrimidine) |
| 506 | (CH₂)₃ | | H | c-Pr | CF₃ | H | |
| 507 | OMe | H | H | CH(Cl)Me | CF₃ | H | 6.15 (s, 5-H, pyrimidine) |
| 508 | OMe | H | H | CH(Cl)Me | CF₃ | 4-F | 6.17 (s, 5-H, pyrimidine) |
| 509 | OMe | H | H | i-Pr | CF₃ | H | 6.08 (s, 5-H, pyrimidine) |
| 510 | OMe | H | H | i-Pr | CF₃ | 4-F | 6.08 (s, 5-H, pyrimidine) |
| 511 | OMe | H | H | CF₃ | CF₃ | H | 6.15 (s, 5-H, pyrimidine) |
| 512 | OMe | H | H | CF₃ | CF₃ | 4-F | 6.18 (s, 5-H, pyrimidine) |
| 513 | OMe | H | H | c-Bu | CF₃ | H | 6.05 (s, 5-H, pyrimidine) |
| 514 | OMe | H | H | c-Bu | CF₃ | 4-F | 6.08 (s, 5-H, pyrimidine) |
| 515 | OMe | H | H | i-Bu | CF₃ | H | 6.05 (s, 5-H, pyrimidine) |
| 516 | OMe | H | H | i-Bu | CF₃ | 4-F | 6.07 (s, 5-H, pyrimidine) |
| 517 | OMe | H | H | Et | CF₃ | H | 6.05 (s, 5-H, pyrimidine) |
| 518 | OMe | H | H | c-Pr | CF₃ | 4-F | 6.06 (s, 5-H, pyrimidine) |
| 519 | OMe | H | H | c-Pr | CF₃ | H | 6.05 (s, 5-H, pyrimidine) |

TABLE 2
Compounds of the formula (I) according to the invention in which A is
A2 and X¹, X² are each hydrogen

| | |
|---|---|
| | (I) |
| 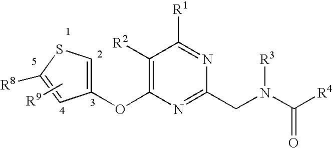 | |

| No. | R¹ | R² | R³ | R⁴ | R⁸ | R⁹ | ¹H-NMR: δ[CDCl₃] |
|---|---|---|---|---|---|---|---|
| 1 | H | H | H | H | H | 4-NO₂ | |
| 2 | H | H | H | Me | CF₃ | H | |
| 3 | H | H | H | Et | CN | H | |
| 4 | H | H | H | Pr | CF₃ | H | |
| 5 | H | H | H | i-Pr | CF₃ | 2-CF₃ | |
| 6 | H | H | H | c-Pr | Cl | 5-Cl | |
| 7 | H | H | H | Bu | CF₃ | H | |
| 8 | H | H | H | i-Bu | H | 4-NO₂ | |
| 9 | H | H | H | c-Bu | CF₃ | H | |
| 10 | H | H | H | t-Bu | CN | H | |
| 11 | H | H | H | c-hexyl | CF₃ | H | |
| 12 | H | H | H | CH₂CH═CH₂ | CF₃ | 2-CF₃ | |
| 13 | H | H | H | CH═CHCH₃ | Cl | 4-Cl | |
| 14 | H | H | H | CH═CH₂ | CF₃ | H | |
| 15 | H | H | H | CH₂C≡CH | H | 2-NO₂ | |
| 16 | H | H | H | C≡CCH₃ | CF₃ | H | |
| 17 | H | H | H | CH₂-c-Pr | CN | H | |
| 18 | H | H | H | CH₂-c-hexyl | CF₃ | H | |
| 19 | H | H | H | CF₃ | CF₃ | 2-CF₃ | |
| 20 | H | H | H | CHFCH₂CH₃ | Cl | 4-Cl | |
| 21 | H | H | H | CHClCH₃ | CF₃ | H | |
| 22 | H | H | H | CH₂OCH₃ | H | 4-NO₂ | |
| 23 | H | H | H | CH₂OCH₂CH₃ | CF₃ | H | |
| 24 | H | H | H | CF(CH₃)₂ | CN | H | |
| 25 | H | H | H | 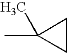 | CF₃ | H | |
| 26 | H | H | Me | H | CF₃ | 2-CF₃ | |
| 27 | H | H | Me | Me | Cl | 4-Cl | |
| 28 | H | H | Me | Et | CF₃ | H | |
| 29 | H | H | Me | Pr | H | 4-NO₂ | |
| 30 | H | H | Me | i-Pr | CF₃ | H | |
| 31 | H | H | Me | c-Pr | CN | H | |
| 32 | H | H | Me | Bu | CF₃ | H | |
| 33 | H | H | Me | i-Bu | CF₃ | 2-CF₃ | |
| 34 | H | H | Me | c-Bu | Cl | 4-Cl | |
| 35 | H | H | Me | t-Bu | CF₃ | H | |
| 36 | H | H | Me | c-hexyl | H | 4-NO₂ | |
| 37 | H | H | Me | CH₂CH═CH₂ | CF₃ | H | |
| 38 | H | H | Me | CH═CHCH₃ | CN | H | |
| 39 | H | H | Me | CH═CH₂ | CF₃ | H | |
| 40 | H | H | Me | CH₂C≡CH | CF₃ | 2-CF₃ | |
| 41 | H | H | Me | C≡CCH₃ | Cl | 4-Cl | |
| 42 | H | H | Me | CH₂-c-Pr | CF₃ | H | |
| 43 | H | H | Me | CH₂-c-hexyl | H | 4-NO₂ | |
| 44 | H | H | Me | CF₃ | CF₃ | H | |
| 45 | H | H | Me | CHFCH₂CH₃ | CN | H | |
| 46 | H | H | Me | CHClCH₃ | CF₃ | H | |
| 47 | H | H | Me | CH₂OCH₃ | CF₃ | 4-CF₃ | |
| 48 | H | H | Me | CH₂OCH₂CH₃ | Cl | 2-Cl | |
| 49 | H | H | Me | CF(CH₃)₂ | CF₃ | H | |
| 50 | H | H | Me | 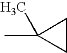 | H | 2-NO₂ | |
| 51 | H | H | CO—Me | H | CF₃ | H | |
| 52 | H | H | CO—Me | Me | CN | H | |
| 53 | H | H | CO—Me | Et | CF₃ | H | |
| 54 | H | H | CO—Me | Pr | CF₃ | 4-CF₃ | |
| 55 | H | H | CO—Me | i-Pr | Cl | 2-Cl | |
| 56 | H | H | CO—Me | c-Pr | CF₃ | H | |
| 57 | H | H | CO—Me | Bu | H | 2-NO₂ | |
| 58 | H | H | CO—Me | i-Bu | CF₃ | H | |
| 59 | H | H | CO—Me | c-Bu | CN | H | |
| 60 | H | H | CO—Me | t-Bu | CF₃ | H | |
| 61 | H | H | CO—Me | c-hexyl | CF₃ | 2-CF₃ | |
| 62 | H | H | CO—Me | CH₂CH═CH₂ | Cl | 4-Cl | |
| 63 | H | H | CO—Me | CH═CHCH₃ | CF₃ | H | |
| 64 | H | H | CO—Me | CH═CH₂ | H | 4-NO₂ | |
| 65 | H | H | CO—Me | CH₂C≡CH | CF₃ | H | |
| 66 | H | H | CO—Me | C≡CCH₃ | CN | H | |
| 67 | H | H | CO—Me | CH₂-c-Pr | CF₃ | H | |
| 68 | H | H | CO—Me | CH₂-c-hexyl | CF₃ | 4-CF₃ | |
| 69 | H | H | CO—Me | CF₃ | Cl | 2-Cl | |
| 70 | H | H | CO—Me | CHFCH₂CH₃ | CF₃ | H | |
| 71 | H | H | CO—Me | CHClCH₃ | H | 2-NO₂ | |
| 72 | H | H | CO—Me | CH₂OCH₃ | CF₃ | H | |
| 73 | H | H | CO—Me | CH₂OCH₂CH₃ | CN | H | |
| 74 | H | H | CO—Me | CF(CH₃)₂ | CF₃ | H | |
| 75 | H | H | CO—Me | 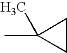 | CF₃ | 2-CF₃ | |
| 76 | H | H | CO-c-Pr | H | Cl | 4-Cl | |
| 77 | H | H | CO-c-Pr | Me | CF₃ | H | |
| 78 | H | H | CO-c-Pr | Et | H | 4-NO₂ | |
| 79 | H | H | CO-c-Pr | Pr | CF₃ | H | |
| 80 | H | H | CO-c-Pr | i-Pr | CN | H | |
| 81 | H | H | CO-c-Pr | c-Pr | CF₃ | H | |
| 82 | H | H | CO-c-Pr | Bu | CF₃ | 4-CF₃ | |
| 83 | H | H | CO-c-Pr | i-Bu | Cl | 2-Cl | |
| 84 | H | H | CO-c-Pr | c-Bu | CF₃ | H | |
| 85 | H | H | CO-c-Pr | t-Bu | H | 2-NO₂ | |
| 86 | H | H | CO-c-Pr | c-hexyl | CF₃ | H | |
| 87 | H | H | CO-c-Pr | CH₂CH═CH₂ | CN | H | |
| 88 | H | H | CO-c-Pr | CH═CHCH₃ | CF₃ | H | |
| 89 | H | H | CO-c-Pr | CH═CH₂ | CF₃ | 2-CF₃ | |
| 90 | H | H | CO-c-Pr | CH₂C≡CH | Cl | 4-Cl | |
| 91 | H | H | CO-c-Pr | C≡CCH₃ | CF₃ | H | |
| 92 | H | H | CO-c-Pr | CH₂-c-Pr | H | 4-NO₂ | |
| 93 | H | H | CO-c-Pr | CH₂-c-hexyl | CF₃ | H | |
| 94 | H | H | CO-c-Pr | CF₃ | CN | H | |
| 95 | H | H | CO-c-Pr | CHFCH₂CH₃ | CF₃ | H | |
| 96 | H | H | CO-c-Pr | CHClCH₃ | CF₃ | 4-CF₃ | |
| 97 | H | H | CO-c-Pr | CH₂OCH₃ | Cl | 2-Cl | |
| 98 | H | H | CO-c-Pr | CH₂OCH₂CH₃ | CF₃ | H | |
| 99 | H | H | CO-c-Pr | CF(CH₃)₂ | H | 2-NO₂ | |
| 100 | H | H | CO-c-Pr | 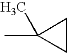 | CF₃ | H | |
| 101 | Cl | H | H | H | CN | H | |
| 102 | Cl | H | H | Me | CF₃ | H | |
| 103 | Cl | H | H | Et | CF₃ | 2-CF₃ | |
| 104 | Cl | H | H | Pr | Cl | 4-Cl | |
| 105 | Cl | H | H | i-Pr | CF₃ | H | |
| 106 | Cl | H | H | c-Pr | H | 4-NO₂ | |
| 107 | Cl | H | H | Bu | CF₃ | H | |
| 108 | Cl | H | H | i-Bu | CN | H | |
| 109 | Cl | H | H | c-Bu | CF₃ | H | |
| 110 | Cl | H | H | t-Bu | CF₃ | 4-CF₃ | |
| 111 | Cl | H | H | c-hexyl | Cl | 2-Cl | |
| 112 | Cl | H | H | CH₂CH═CH₂ | CF₃ | H | |
| 113 | Cl | H | H | CH═CHCH₃ | H | 2-NO₂ | |
| 114 | Cl | H | H | CH═CH₂ | CF₃ | H | |
| 115 | Cl | H | H | CH₂C≡CH | CN | H | |
| 116 | Cl | H | H | C≡CCH₃ | CF₃ | H | |
| 117 | Cl | H | H | CH₂-c-Pr | CF₃ | 2-CF₃ | |
| 118 | Cl | H | H | CH₂-c-hexyl | Cl | 4-Cl | |
| 119 | Cl | H | H | CF₃ | CF₃ | H | |
| 120 | Cl | H | H | CHFCH₂CH₃ | H | 4-NO₂ | |
| 121 | Cl | H | H | CHClCH₃ | CF₃ | H | |
| 122 | Cl | H | H | CH₂OCH₃ | ON | H | |
| 123 | Cl | H | H | CH₂OCH₂CH₃ | CF₃ | H | |
| 124 | Cl | H | H | CF(CH₃)₂ | CF₃ | 4-CF₃ | |
| 125 | Cl | H | H | 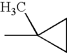 | Cl | 2-Cl | |
| 126 | Cl | H | Me | H | CF₃ | H | |
| 127 | Cl | H | Me | Me | H | 2-NO₂ | |
| 128 | Cl | H | Me | Et | CF₃ | H | |
| 129 | Cl | H | Me | Pr | CN | H | |
| 130 | Cl | H | Me | i-Pr | CF₃ | H | |
| 131 | Cl | H | Me | c-Pr | CF₃ | 2-CF₃ | |
| 132 | Cl | H | Me | Bu | Cl | 4-Cl | |
| 133 | Cl | H | Me | i-Bu | CF₃ | H | |
| 134 | Cl | H | Me | c-Bu | H | 4-NO₂ | |
| 135 | Cl | H | Me | t-Bu | CF₃ | H | |
| 136 | Cl | H | Me | c-hexyl | CN | H | |
| 137 | Cl | H | Me | CH₂CHCH₂ | CF₃ | H | |
| 138 | Cl | H | Me | CH═CHCH₃ | CF₃ | 4-CF₃ | |
| 139 | Cl | H | Me | CH═CH₂ | Cl | 2-Cl | |
| 140 | Cl | H | Me | CH₂C≡CH | CF₃ | H | |
| 141 | Cl | H | Me | C≡CCH₃ | H | 2-NO₂ | |
| 142 | Cl | H | Me | CH₂-c-Pr | CF₃ | H | |
| 143 | Cl | H | Me | CH₂-c-hexyl | CN | H | |
| 144 | Cl | H | Me | CF₃ | CF₃ | H | |
| 145 | Cl | H | Me | CHFCH₂CH₃ | CF₃ | 2-CF₃ | |
| 146 | Cl | H | Me | CHClCH₃ | Cl | 4-Cl | |
| 147 | Cl | H | Me | CH₂OCH₃ | CF₃ | H | |
| 148 | Cl | H | Me | CH₂OCH₂CH₃ | H | 4-NO₂ | |
| 149 | Cl | H | Me | CF(CH₃)₂ | CF₃ | H | |
| 150 | Cl | H | Me | 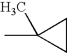 | CN | H | |
| 151 | Cl | H | CO—Me | H | CF₃ | H | |
| 152 | Cl | H | CO—Me | Me | CF₃ | 4-CF₃ | |
| 153 | Cl | H | CO—Me | Et | Cl | 2-Cl | |
| 154 | Cl | H | CO—Me | Pr | CF₃ | H | |
| 155 | Cl | H | CO—Me | i-Pr | H | 2-NO₂ | |
| 156 | Cl | H | CO—Me | c-Pr | CF₃ | H | |
| 157 | Cl | H | CO—Me | Bu | CN | H | |
| 158 | Cl | H | CO—Me | i-Bu | CF₃ | H | |
| 159 | Cl | H | CO—Me | c-Bu | CF₃ | 2-CF₃ | |
| 160 | Cl | H | CO—Me | t-Bu | Cl | 4-Cl | |
| 161 | Cl | H | CO—Me | c-hexyl | CF₃ | H | |
| 162 | Cl | H | CO—Me | CH₂CHCH₂ | H | 4-NO₂ | |
| 163 | Cl | H | CO—Me | CHCHCH₃ | CF₃ | H | |
| 164 | Cl | H | CO—Me | CH═CH₂ | CN | H | |
| 165 | Cl | H | CO—Me | CH₂C≡CH | CF₃ | H | |
| 166 | Cl | H | CO—Me | C≡CCH₃ | CF₃ | 4-CF₃ | |
| 167 | Cl | H | CO—Me | CH₂-c-Pr | Cl | 2-Cl | |
| 168 | Cl | H | CO—Me | CH₂-c-hexyl | CF₃ | H | |
| 169 | Cl | H | CO—Me | CF₃ | H | 2-NO₂ | |
| 170 | Cl | H | CO—Me | CHFCH₂CH₃ | CF₃ | H | |
| 171 | Cl | H | CO—Me | CHClCH₃ | CN | H | |
| 172 | Cl | H | CO—Me | CH₂OCH₃ | CF₃ | H | |
| 173 | Cl | H | CO—Me | CH₂OCH₂CH₃ | CF₃ | 4-CF₃ | |
| 174 | Cl | H | CO—Me | CF(CH₃)₂ | Cl | 2-Cl | |
| 175 | Cl | H | CO—Me | 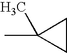 | CF₃ | H | |
| 176 | Cl | H | CO-c-Pr | H | H | 2-NO₂ | |
| 177 | Cl | H | CO-c-Pr | Me | CF₃ | H | |
| 178 | Cl | H | CO-c-Pr | Et | CN | H | |
| 179 | Cl | H | CO-c-Pr | Pr | CF₃ | H | |
| 180 | Cl | H | CO-c-Pr | i-Pr | CF₃ | 2-CF₃ | |
| 181 | Cl | H | CO-c-Pr | c-Pr | Cl | 4-Cl | |
| 182 | Cl | H | CO-c-Pr | Bu | CF₃ | H | |
| 183 | Cl | H | CO-c-Pr | i-Bu | H | 4-NO₂ | |
| 184 | Cl | H | CO-c-Pr | c-Bu | CF₃ | H | |
| 185 | Cl | H | CO-c-Pr | t-Bu | CN | H | |
| 186 | Cl | H | CO-c-Pr | c-hexyl | CF₃ | H | |
| 187 | Cl | H | CO-c-Pr | CH₂CH═CH₂ | CF₃ | 4-CF₃ | |
| 188 | Cl | H | CO-c-Pr | CH═CHCH₃ | Cl | 2-Cl | |
| 189 | Cl | H | CO-c-Pr | CHCH₂ | CF₃ | H | |
| 190 | Cl | H | CO-c-Pr | CH₂C≡CH | H | 2-NO₂ | |
| 191 | Cl | H | CO-c-Pr | C≡CCH₃ | CF₃ | H | |
| 192 | Cl | H | CO-c-Pr | CH₂-c-Pr | CN | H | |
| 193 | Cl | H | CO-c-Pr | CH₂-c-hexyl | CF₃ | H | |
| 194 | Cl | H | CO-c-Pr | CF₃ | CF₃ | 2-CF₃ | |
| 195 | Cl | H | CO-c-Pr | CHFCH₂CH₃ | Cl | 4-Cl | |
| 196 | Cl | H | CO-c-Pr | CHClCH₃ | CF₃ | H | |
| 197 | Cl | H | CO-c-Pr | CH₂OCH₃ | H | 4-NO₂ | |
| 198 | Cl | H | CO-c-Pr | CH₂OCH₂CH₃ | CF₃ | H | |
| 199 | Cl | H | CO-c-Pr | CF(CH₃)₂ | CN | H | |
| 200 | Cl | H | CO-c-Pr | 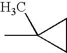 | CF₃ | H | |
| 201 | H | Me | H | H | CF₃ | 4-CF₃ | |
| 202 | H | Me | H | Me | Cl | 2-Cl | |
| 203 | H | Me | H | Et | CF₃ | H | |
| 204 | Me | H | H | i-Pr | CF₃ | H | 6.64 (s, 5-H, pyrimidine) |
| 205 | Me | H | H | c-Pr | CF₃ | H | 6.80 (s, 5-H, pyrimidine) |
| 206 | H | Me | H | c-Pr | CF₃ | H | 0.75 (m, 2H), 0.92 (m, 2H), 1.40 |
| | | | | | | | (m, 1H), 2.30 (s, 1H), |
| | | | | | | | 4.58 (d, 2H), |
| | | | | | | | 6.65 (bs, 1H), 7.38 (m, 1H), 7.40 |
| | | | | | | | (m, 1H), 8.20 (s, 1H). |
| 207 | H | Me | H | Bu | CF₃ | H | |
| 208 | H | Me | H | i-Bu | CF₃ | 2-CF₃ | |
| 209 | Me | H | H | c-Bu | Cl | 4-Cl | |
| 210 | H | Me | H | t-Bu | CF₃ | H | |
| 211 | H | Me | H | c-hexyl | H | 4-NO₂ | |
| 212 | H | Me | H | CH₂CHCH₂ | CF₃ | H | |
| 213 | H | Me | H | CHCHCH₃ | CN | H | |
| 214 | H | Me | H | CH═CH₂ | CF₃ | H | |
| 215 | H | Me | H | CH₂C≡CH | CF₃ | 4-CF₃ | |
| 216 | H | Me | H | C≡CCH₃ | Cl | 2-Cl | |
| 217 | H | Me | H | CH₂-c-Pr | CF₃ | H | |
| 218 | H | Me | H | CH₂-c-hexyl | H | 2-NO₂ | |
| 219 | H | Me | H | CF₃ | CF₃ | H | 6.72 (s, 5-H, pyrimidine) |
| 220 | H | Me | H | CHFCH₂CH₃ | CN | H | |
| 221 | H | Me | H | CHClCH₃ | CF₃ | H | |
| 222 | H | Me | H | CH₂OCH₃ | CF₃ | 2-CF₃ | |
| 223 | H | Me | H | CH₂OCH₂CH₃ | Cl | 4-Cl | |
| 224 | H | Me | H | CF(CH₃)₂ | CF₃ | H | |
| 225 | H | Me | H | 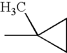 | H | 4-NO₂ | |
| 226 | H | Me | Me | H | CF₃ | H | |
| 227 | H | Me | Me | Me | CN | H | |
| 228 | H | Me | Me | Et | CF₃ | H | |
| 229 | H | Me | Me | Pr | CF₃ | 4-CF₃ | |
| 230 | H | Me | Me | i-Pr | Cl | 2-Cl | |
| 231 | H | Me | Me | c-Pr | CF₃ | H | |
| 232 | H | Me | Me | Bu | H | 2-NO₂ | |
| 233 | H | Me | Me | i-Bu | CF₃ | H | |
| 234 | H | Me | Me | c-Bu | CN | H | |
| 235 | H | Me | Me | t-Bu | CF₃ | H | |
| 236 | H | Me | Me | c-hexyl | CF₃ | 2-CF₃ | |
| 237 | H | Me | Me | CH₂C≡CH₂ | Cl | 4-Cl | |
| 238 | H | Me | Me | CH═CHCH₃ | CF₃ | H | |
| 239 | H | Me | Me | CH═CH₂ | H | 4-NO₂ | |
| 240 | H | Me | Me | CH₂C≡CH | CF₃ | H | |
| 241 | H | Me | Me | C≡CCH₃ | CN | H | |
| 242 | H | Me | Me | CH₂-c-Pr | CF₃ | H | |
| 243 | H | Me | Me | CH₂-c-hexyl | CF₃ | 4-CF₃ | |
| 244 | H | Me | Me | CF₃ | Cl | 2-Cl | |
| 245 | H | Me | Me | CHFCH₂CH₃ | CF₃ | H | |
| 246 | H | Me | Me | CHClCH₃ | H | 2-NO₂ | |
| 247 | H | Me | Me | CH₂OCH₃ | CF₃ | H | |
| 248 | H | Me | Me | CH₂OCH₂CH₃ | CN | H | |
| 249 | H | Me | Me | CF(CH₃)₂ | CF₃ | H | |
| 250 | H | Me | Me | 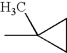 | CF₃ | 2-CF₃ | |
| 251 | H | Me | CO—Me | H | Cl | 4-Cl | |
| 252 | H | Me | CO—Me | Me | CF₃ | H | |
| 253 | H | Me | CO—Me | Et | H | 4-NO₂ | |
| 254 | H | Me | CO—Me | Pr | CF₃ | H | |
| 255 | H | Me | CO—Me | i-Pr | CN | H | |
| 256 | H | Me | CO—Me | c-Pr | CF₃ | H | |
| 257 | H | Me | CO—Me | Bu | CF₃ | 4-CF₃ | |
| 258 | H | Me | CO—Me | i-Bu | Cl | 2-Cl | |
| 259 | H | Me | CO—Me | c-Bu | CF₃ | H | |
| 260 | H | Me | CO—Me | t-Bu | H | 2-NO₂ | |
| 261 | H | Me | CO—Me | c-hexyl | CF₃ | H | |
| 262 | H | Me | CO—Me | CH₂CHCH₂ | CN | H | |
| 263 | H | Me | CO—Me | CH═CHCH₃ | CF₃ | H | |
| 264 | H | Me | CO—Me | CH═CH₂ | CF₃ | 2-CF₃ | |
| 265 | H | Me | CO—Me | CH₂C≡CH | Cl | 4-Cl | |
| 266 | H | Me | CO—Me | C≡CCH₃ | CF₃ | H | |
| 267 | H | Me | CO—Me | CH₂-c-Pr | H | 4-NO₂ | |
| 268 | H | Me | CO—Me | CH₂-c-hexyl | CF₃ | H | |
| 269 | H | Me | CO—Me | CF₃ | CN | H | |
| 270 | H | Me | CO—Me | CHFCH₂CH₃ | CF₃ | H | |
| 271 | H | Me | CO—Me | CHClCH₃ | CF₃ | 4-CF₃ | |
| 272 | H | Me | CO—Me | CH₂OCH₃ | Cl | 2-Cl | |
| 273 | H | Me | CO—Me | CH₂OCH₂CH₃ | CF₃ | H | |
| 274 | H | Me | CO—Me | CF(CH₃)₂ | H | 2-NO₂ | |
| 275 | H | Me | CO—Me | 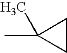 | CF₃ | H | |
| 276 | H | Me | CO-c-Pr | H | CN | H | |
| 277 | H | Me | CO-c-Pr | Me | CF₃ | H | |
| 278 | H | Me | CO-c-Pr | Et | CF₃ | 2-CF₃ | |
| 279 | H | Me | CO-c-Pr | Pr | Cl | 4-Cl | |
| 280 | H | Me | CO-c-Pr | i-Pr | CF₃ | H | |
| 281 | H | Me | CO-c-Pr | c-Pr | H | 4-NO₂ | |
| 282 | H | Me | CO-c-Pr | Bu | CF₃ | H | |
| 283 | H | Me | CO-c-Pr | i-Bu | CN | H | |
| 284 | H | Me | CO-c-Pr | c-Bu | CF₃ | H | |
| 285 | H | Me | CO-c-Pr | t-Bu | CF₃ | 4-CF₃ | |
| 286 | H | Me | CO-c-Pr | c-hexyl | Cl | 2-Cl | |
| 287 | H | Me | CO-c-Pr | CH₂CHCH₂ | CF₃ | H | |
| 288 | H | Me | CO-c-Pr | CHCHCH₃ | H | 2-NO₂ | |
| 289 | H | Me | CO-c-Pr | CH═CH₂ | CF₃ | H | |
| 290 | H | Me | CO-c-Pr | CH₂C≡CH | CN | H | |
| 291 | H | Me | CO-c-Pr | C≡CCH₃ | CF₃ | H | |
| 292 | H | Me | CO-c-Pr | CH₂-c-Pr | CF₃ | 2-CF₃ | |
| 293 | H | Me | CO-c-Pr | CH₂-c-hexyl | Cl | 4-Cl | |
| 294 | H | Me | CO-c-Pr | CF₃ | CF₃ | H | |
| 295 | H | Me | CO-c-Pr | CHFCH₂CH₃ | H | 4-NO₂ | |
| 296 | H | Me | CO-c-Pr | CHClCH₃ | CF₃ | H | |
| 297 | H | Me | CO-c-Pr | CH₂OCH₃ | CN | H | |
| 298 | H | Me | CO-c-Pr | CH₂OCH₂CH₃ | CF₃ | H | |
| 299 | H | Me | CO-c-Pr | CF(CH₃)₂ | CF₃ | 4-CF₃ | |
| 300 | H | Me | CO-c-Pr | 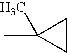 | Cl | 2-Cl | |
| 301 | Et | H | H | H | CF₃ | H | |
| 302 | Et | H | H | Me | H | 2-NO₂ | |
| 303 | Et | H | H | Et | CF₃ | H | |
| 304 | Et | H | H | c-Pr | CF₃ | H | 6.72 (s, 5-H, pyrimidine) |
| 305 | H | Et | H | i-Pr | CF₃ | H | 8.40 (s, 6-H, pyrimidine) |
| 306 | H | Et | H | c-Pr | CF₃ | H | |
| 307 | Et | H | H | Bu | Cl | 4-Cl | |
| 308 | Et | H | H | i-Bu | CF₃ | H | |
| 309 | Et | H | H | c-Bu | H | 4-NO₂ | |
| 310 | Et | H | H | t-Bu | CF₃ | H | 6.65 (s, 5-H, pyrimidine) |
| 311 | Et | H | H | c-hexyl | CN | H | |
| 312 | Et | H | H | CH₂CH≡CH₂ | CF₃ | H | |
| 313 | Et | H | H | CH═CHCH₃ | CF₃ | 4-CF₃ | |
| 314 | Et | H | H | CH≡CH₂ | Cl | 2-Cl | |
| 315 | Et | H | H | CH₂C≡CH | CF₃ | H | |
| 316 | Et | H | H | C≡CCH₃ | H | 2-NO₂ | |
| 317 | Et | H | H | CH₂-c-Pr | CF₃ | H | |
| 318 | Et | H | H | CH₂-c-hexyl | CN | H | |
| 319 | Et | H | H | CF₃ | CF₃ | H | 6.75 (s, 5-H, pyrimidine) |
| 320 | Et | H | H | CHFCH₂CH₃ | CF₃ | 2-CF₃ | |
| 321 | Et | H | H | CHClCH₃ | Cl | 4-Cl | |
| 322 | Et | H | H | CH₂OCH₃ | CF₃ | H | |
| 323 | Et | H | H | CH₂OCH₂CH₃ | H | 4-NO₂ | |
| 324 | Et | H | H | CF(CH₃)₂ | CF₃ | H | 6.60 (s, 5-H, pyrimidine) |
| 325 | Et | H | H | 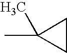 | CF₃ | H | 6.70 (s, 5-H, pyrimidine) |
| 326 | Et | H | Me | H | CF₃ | H | |
| 327 | (CH₂)₃ | | H | c-Pr | CF₃ | H | |
| 328 | OMe | H | H | CH(Cl)Me | CF₃ | H | 6.15 (s, 5-H, pyrimidine) |
| 329 | OMe | H | H | i-Pr | CF₃ | H | 6.10 (s, 5-H, pyrimidine) |
| 330 | OMe | H | H | CF₃ | CF₃ | H | 6.17 (s, 5-H, pyrimidine) |
| 331 | OMe | H | H | c-Bu | CF₃ | H | 6.10 (s, 5-H, pyrimidine) |
| 332 | OMe | H | H | i-Bu | CF₃ | H | 6.05 (s, 5-H, pyrimidine) |
| 333 | OMe | H | H | Et | CF₃ | H | 6.10 (s, 5-H, pyrimidine) |

TABLE 3
Compounds of the formula (I) according to the invention in which A is
A3 and X¹, X² are each hydrogen

| | |
|---|---|
| | (I) |
| 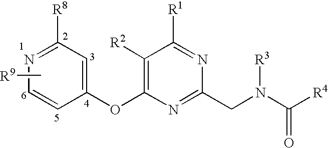 | |

| No. | R¹ | R² | R³ | R⁴ | R⁸ | R⁹ | ¹H-NMR: δ[CDCl₃] |
|---|---|---|---|---|---|---|---|
| 1 | H | H | H | H | H | 6-NO₂ | |
| 2 | H | H | H | Me | CF₃ | H | |
| 3 | H | H | H | Et | CN | H | |
| 4 | H | H | H | Pr | CF₃ | H | |
| 5 | H | H | H | i-Pr | CF₃ | 6-CF₃ | |
| 6 | H | H | H | c-Pr | Cl | 6-Cl | |
| 7 | H | H | H | Bu | CF₃ | H | |
| 8 | H | H | H | i-Bu | H | 3-NO₂ | |
| 9 | H | H | H | c-Bu | CF₃ | H | |
| 10 | H | H | H | t-Bu | CN | H | |
| 11 | H | H | H | c-hexyl | CF₃ | H | |
| 12 | H | H | H | CH₂CH═CH₂ | CF₃ | 3-CF₃ | |
| 13 | H | H | H | CH═CHCH₃ | Cl | 3-Cl | |
| 14 | H | H | H | CH═CH₂ | CF₃ | H | |
| 15 | H | H | H | CH₂C═CH | H | 5-NO₂ | |
| 16 | H | H | H | C≡CCH₃ | CF₃ | H | |
| 17 | H | H | H | CH₂-c-Pr | CN | H | |
| 18 | H | H | H | CH₂-c-hexyl | CF₃ | H | |
| 19 | H | H | H | CF₃ | CF₃ | H | 7.00, 8.60(2d, 5-H and 6-H, |
| | | | | | | | pyrimidine) |
| 20 | H | H | H | CHFCH₂CH₃ | Cl | 5-Cl | |
| 21 | H | H | H | CHClCH₃ | CF₃ | H | |
| 22 | H | H | H | CH₂OCH₃ | H | 5-NO₂ | |
| 23 | H | H | H | CH₂OCH₂CH₃ | CF₃ | H | |
| 24 | H | H | H | CF(CH₃)₂ | CN | H | |
| 25 | H | H | H | 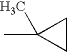 | CF₃ | H | |
| 26 | H | H | Me | H | CF₃ | 3-CF₃ | |
| 27 | H | H | Me | Me | Cl | 6-Cl | |
| 28 | H | H | Me | Et | CF₃ | H | |
| 29 | H | H | Me | Pr | H | 3-NO₂ | |
| 30 | H | H | Me | i-Pr | CF₃ | H | |
| 31 | H | H | Me | c-Pr | CN | H | |
| 32 | H | H | Me | Bu | CF₃ | H | |
| 33 | H | H | Me | i-Bu | CF₃ | 3-CF₃ | |
| 34 | H | H | Me | c-Bu | Cl | 3-Cl | |
| 35 | H | H | Me | t-Bu | CF₃ | H | |
| 36 | H | H | Me | c-hexyl | H | 6-NO₂ | |
| 37 | H | H | Me | CH₂CH═CH₂ | CF₃ | H | |
| 38 | H | H | Me | CH═CHCH₃ | CN | H | |
| 39 | H | H | Me | CH═CH₂ | CF₃ | H | |
| 40 | H | H | Me | CH₂C═CH | CF₃ | 5-CF₃ | |
| 41 | H | H | Me | C≡CCH₃ | Cl | 5-Cl | |
| 42 | H | H | Me | CH₂-c-Pr | CF₃ | H | |
| 43 | H | H | Me | CH₂-c-hexyl | H | 5-NO₂ | |
| 44 | H | H | Me | CF₃ | CF₃ | H | |
| 45 | H | H | Me | CHFCH₂CH₃ | CN | H | |
| 46 | H | H | Me | CHClCH₃ | CF₃ | H | |
| 47 | H | H | Me | CH₂OCH₃ | CF₃ | 5-CF₃ | |
| 48 | H | H | Me | CH₂OCH₂CH₃ | Cl | 5-Cl | |
| 49 | H | H | Me | CF(CH₃)₂ | CF₃ | H | |
| 50 | H | H | Me | 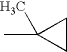 | H | 5-NO₂ | |
| 51 | H | H | CO-Me | H | CF₃ | H | |
| 52 | H | H | CO-Me | Me | CN | H | |
| 53 | H | H | CO-Me | Et | CF₃ | H | |
| 54 | H | H | CO-Me | Pr | CF₃ | 6-CF₃ | |
| 55 | H | H | CO-Me | i-Pr | Cl | 6-Cl | |
| 56 | H | H | CO-Me | c-Pr | CF₃ | H | |
| 57 | H | H | CO-Me | Bu | H | 6-NO₂ | |
| 58 | H | H | CO-Me | i-Bu | CF₃ | H | |
| 59 | H | H | CO-Me | c-Bu | CN | H | |
| 60 | H | H | CO-Me | t-Bu | CF₃ | H | |
| 61 | H | H | CO-Me | c-hexyl | CF₃ | 6-CF₃ | |
| 62 | H | H | CO-Me | CH₂CH═CH₂ | Cl | 6-Cl | |
| 63 | H | H | CO-Me | CH═CHCH₃ | CF₃ | H | |
| 64 | H | H | CO-Me | CH═CH₂ | H | 6-NO₂ | |
| 65 | H | H | CO-Me | CH₂C≡CH | CF₃ | H | |
| 66 | H | H | CO-Me | C≡CCH₃ | CN | H | |
| 67 | H | H | CO-Me | CH₂-c-Pr | CF₃ | H | |
| 68 | H | H | CO-Me | CH₂-c-hexyl | CF₃ | 5-CF₃ | |
| 69 | H | H | CO-Me | CF₃ | Cl | 5-Cl | |
| 70 | H | H | CO-Me | CHFCH₂CH₃ | CF₃ | H | |
| 71 | H | H | CO-Me | CHClCH₃ | H | 5-NO₂ | |
| 72 | H | H | CO-Me | CH₂OCH₃ | CF₃ | H | |
| 73 | H | H | CO-Me | CH₂OCH₂CH₃ | CN | H | |
| 74 | H | H | CO-Me | CF(CH₃)₂ | CF₃ | H | |
| 75 | H | H | CO-Me | 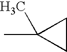 | CF₃ | 6-CF₃ | |
| 76 | H | H | CO-c-Pr | H | Cl | 6-Cl | |
| 77 | H | H | CO-c-Pr | Me | CF₃ | H | |
| 78 | H | H | CO-c-Pr | Et | H | 6-NO₂ | |
| 79 | H | H | CO-c-Pr | Pr | CF₃ | H | |
| 80 | H | H | CO-c-Pr | i-Pr | CN | H | |
| 81 | H | H | CO-c-Pr | c-Pr | CF₃ | H | |
| 82 | H | H | CO-c-Pr | Bu | CF₃ | 5-CF₃ | |
| 83 | H | H | CO-c-Pr | i-Bu | Cl | 5-Cl | |
| 84 | H | H | CO-c-Pr | c-Bu | CF₃ | H | |
| 85 | H | H | CO-c-Pr | t-Bu | H | 5-NO₂ | |
| 86 | H | H | CO-c-Pr | c-hexyl | CF₃ | H | |
| 87 | H | H | CO-c-Pr | CH₂CH═CH₂ | CN | H | |
| 88 | H | H | CO-c-Pr | CH═CHCH₃ | CF₃ | H | |
| 89 | H | H | CO-c-Pr | CH═CH₂ | CF₃ | 3-CF₃ | |
| 90 | H | H | CO-c-Pr | CH₂C≡CH | Cl | 3-Cl | |
| 91 | H | H | CO-c-Pr | C≡CCH₃ | CF₃ | H | |
| 92 | H | H | CO-c-Pr | CH₂-c-Pr | H | 3-NO₂ | |
| 93 | H | H | CO-c-Pr | CH₂-c-hexyl | CF₃ | H | |
| 94 | H | H | CO-c-Pr | CF₃ | CN | H | |
| 95 | H | H | CO-c-Pr | CHFCH₂CH₃ | CF₃ | H | |
| 96 | H | H | CO-c-Pr | CH | ClCH₃ | CF₃ | 6-CF₃ |
| 97 | H | H | CO-c-Pr | CH₂OCH₃ | Cl | 6-Cl | |
| 98 | H | H | CO-c-Pr | CH₂OCH₂CH₃ | CF₃ | H | |
| 99 | H | H | CO-c-Pr | CF(CH₃)₂ | H | 6-NO₂ | |
| 100 | H | H | CO-c-Pr | 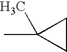 | CF₃ | H | |
| 101 | Cl | H | H | H | CN | H | |
| 102 | Cl | H | H | Me | CF₃ | H | |
| 103 | Cl | H | H | Et | CF₃ | 3-CF₃ | |
| 104 | Cl | H | H | Pr | Cl | 3-Cl | |
| 105 | Cl | H | H | i-Pr | CF₃ | H | |
| 106 | Cl | H | H | c-Pr | H | 3-NO₂ | |
| 107 | Cl | H | H | Bu | CF₃ | H | |
| 108 | Cl | H | H | i-Bu | CN | H | |
| 109 | Cl | H | H | c-Bu | CF₃ | H | |
| 110 | Cl | H | H | t-Bu | CF₃ | 3-CF₃ | |
| 111 | Cl | H | H | c-hexyl | Cl | 3-Cl | |
| 112 | Cl | H | H | CH₂CH═CH₂ | CF₃ | H | |
| 113 | Cl | H | H | CH═CHCH₃ | H | 3-NO₂ | |
| 114 | Cl | H | H | CH═CH₂ | CF₃ | H | |
| 115 | Cl | H | H | CH₂C≡CH | CN | H | |
| 116 | Cl | H | H | C≡CCH₃ | CF₃ | H | |
| 117 | Cl | H | H | CH₂-c-Pr | CF₃ | 6-CF₃ | |
| 118 | Cl | H | H | CH₂-c-hexyl | Cl | 6-Cl | |
| 119 | Cl | H | H | CF₃ | CF₃ | H | |
| 120 | Cl | H | H | CHFCH₂CH₃ | H | 6-NO₂ | |
| 121 | Cl | H | H | CH | ClCH₃ | CF₃ | H |
| 122 | Cl | H | H | CH₂OCH₃ | CN | H | |
| 123 | Cl | H | H | CH₂OCH₂CH₃ | CF₃ | H | |
| 124 | Cl | H | H | CF(CH₃)₂ | CF₃ | 6-CF₃ | |
| 125 | Cl | H | H | 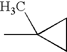 | Cl | 6-Cl | |
| 126 | Cl | H | Me | H | CF₃ | H | |
| 127 | Cl | H | Me | Me | H | 6-NO₂ | |
| 128 | Cl | H | Me | Et | CF₃ | H | |
| 129 | Cl | H | Me | Pr | CN | H | |
| 130 | Cl | H | Me | i-Pr | CF₃ | H | |
| 131 | Cl | H | Me | c-Pr | CF₃ | 5-CF₃ | |
| 132 | Cl | H | Me | Bu | Cl | 5-Cl | |
| 133 | Cl | H | Me | i-Bu | CF₃ | H | |
| 134 | Cl | H | Me | c-Bu | H | 5-NO₂ | |
| 135 | Cl | H | Me | t-Bu | CF₃ | H | |
| 136 | Cl | H | Me | c-hexyl | CN | H | |
| 137 | Cl | H | Me | CH₂CH═CH₂ | CF₃ | H | |
| 138 | Cl | H | Me | CH═CHCH₃ | CF₃ | 3-CF₃ | |
| 139 | Cl | H | Me | CH═CH₂ | Cl | 3-Cl | |
| 140 | Cl | H | Me | CH₂C≡CH | CF₃ | H | |
| 141 | Cl | H | Me | C≡CCH₃ | H | 3-NO₂ | |
| 142 | Cl | H | Me | CH₂-c-Pr | CF₃ | H | |
| 143 | Cl | H | Me | CH₂-c-hexyl | CN | H | |
| 144 | Cl | H | Me | CF₃ | CF₃ | H | |
| 145 | Cl | H | Me | CHFCH₂CH₃ | CF₃ | 6-CF₃ | |
| 146 | Cl | H | Me | CHClCH₃ | Cl | 6-Cl | |
| 147 | Cl | H | Me | CH₂OCH₃ | CF₃ | H | |
| 148 | Cl | H | Me | CH₂OCH₂CH₃ | H | 6-NO₂ | |
| 149 | Cl | H | Me | CF(CH₃)₂ | CF₃ | H | |
| 150 | Cl | H | Me | 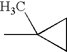 | CN | H | |
| 151 | Cl | H | CO-Me | H | CF₃ | H | |
| 152 | Cl | H | CO-Me | Me | CF₃ | 5-CF₃ | |
| 153 | Cl | H | CO-Me | Et | Cl | 5-Cl | |
| 154 | Cl | H | CO-Me | Pr | CF₃ | H | |
| 155 | Cl | H | CO-Me | i-Pr | H | 5-NO₂ | |
| 156 | Cl | H | CO-Me | c-Pr | CF₃ | H | |
| 157 | Cl | H | CO-Me | Bu | CN | H | |
| 158 | Cl | H | CO-Me | i-Bu | CF₃ | H | |
| 159 | Cl | H | CO-Me | c-Bu | CF₃ | 3-CF₃ | |
| 160 | Cl | H | CO-Me | t-Bu | Cl | 3-Cl | |
| 161 | Cl | H | CO-Me | c-hexyl | CF₃ | H | |
| 162 | Cl | H | CO-Me | CH₂CH═CH₂ | H | 3-NO₂ | |
| 163 | Cl | H | CO-Me | CH═CHCH₃ | CF₃ | H | |
| 164 | Cl | H | CO-Me | CH═CH₂ | CN | H | |
| 165 | Cl | H | CO-Me | CH₂C≡CH | CF₃ | H | |
| 166 | Cl | H | CO-Me | C≡CCH₃ | CF₃ | 6-CF₃ | |
| 167 | Cl | H | CO-Me | CH₂-c-Pr | Cl | 6-Cl | |
| 168 | Cl | H | CO-Me | CH₂-c-hexyl | CF₃ | H | |
| 169 | Cl | H | CO-Me | CF₃ | H | 6-NO₂ | |
| 170 | Cl | H | CO-Me | CHFCH₂CH₃ | CF₃ | H | |
| 171 | Cl | H | CO-Me | CHClCH₃ | CN | H | |
| 172 | Cl | H | CO-Me | CH₂OCH₃ | CF₃ | H | |
| 173 | Cl | H | CO-Me | CH₂OCH₂CH₃ | CF₃ | 6-CF₃ | |
| 174 | Cl | H | CO-Me | CF(CH₃)₂ | Cl | 6-Cl | |
| 175 | Cl | H | CO-Me | 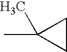 | CF₃ | K | |
| 176 | Cl | H | CO-c-Pr | H | H | 6-NO₂ | |
| 177 | Cl | H | CO-c-Pr | Me | CF₃ | H | |
| 178 | Cl | H | CO-c-Pr | Et | CN | H | |
| 179 | Cl | H | CO-c-Pr | Pr | CF₃ | H | |
| 180 | Cl | H | CO-c-Pr | i-Pr | CF₃ | 6-CF₃ | |
| 181 | Cl | H | CO-c-Pr | c-Pr | Cl | 6-Cl | |
| 182 | Cl | H | CO-c-Pr | Bu | CF₃ | H | |
| 183 | Cl | H | CO-c-Pr | i-Bu | H | 6-NO₂ | |
| 184 | Cl | H | CO-c-Pr | c-Bu | CF₃ | H | |
| 185 | Cl | H | CO-c-Pr | t-Bu | CN | H | |
| 186 | Cl | H | CO-c-Pr | c-hexyl | CF₃ | H | |
| 187 | Cl | H | CO-c-Pr | CH₂CH═CH₂ | CF₃ | 5-CF₃ | |
| 188 | Cl | H | CO-c-Pr | CH═CHCH₃ | Cl | 5-Cl | |
| 189 | Cl | H | CO-c-Pr | CH═CH₂ | CF₃ | H | |
| 190 | Cl | H | CO-c-Pr | CH₂C≡CH | H | 5-NO₂ | |
| 191 | Cl | H | CO-c-Pr | C≡CCH₃ | CF₃ | H | |
| 192 | Cl | H | CO-c-Pr | CH₂-c-Pr | CN | H | |
| 193 | Cl | H | CO-c-Pr | CH₂-c-hexyl | CF₃ | H | |
| 194 | Cl | H | CO-c-Pr | CF₃ | CF₃ | 3-CF₃ | |
| 195 | Cl | H | CO-c-Pr | CHFCH₂CH₃ | Cl | 3-Cl | |
| 196 | Cl | H | CO-c-Pr | CHClCH₃ | CF₃ | H | |
| 197 | Cl | H | CO-c-Pr | CH₂OCH₃ | H | 3-NO₂ | |
| 198 | Cl | H | CO-c-Pr | CH₂OCH₂CH₃ | CF₃ | H | |
| 199 | Cl | H | CO-c-Pr | CF(CH₃)₂ | CN | H | |
| 200 | Cl | H | CO-c-Pr | 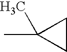 | CF₃ | H | |
| 201 | Me | H | H | H | CF₃ | 6-CF₃ | |
| 202 | Me | H | H | Me | Cl | 6-Cl | |
| 203 | Me | H | H | Et | CF₃ | H | |
| 204 | Me | H | H | i-Pr | CF₃ | H | 6.80(s, 5-H, pyrimidine) |
| 205 | H | Me | H | i-Pr | CF₃ | H | 8.49(s, 6-H, pyrimidine) |
| 206 | Me | H | H | c-Pr | CF₃ | H | 6.79(s, 5-H, pyrimidine) |
| 207 | Me | H | H | i-Pr | Cl | H | 6.75(s, 5-H, pyrimidine) |
| 208 | Me | H | H | c-Pr | Cl | H | 6.75(s, 5-H, pyrimidine) |
| 209 | Me | H | H | c-Bu | CF₃ | H | 6.78(s, 5-H, pyrimidine) |
| 210 | Me | H | H | t-Bu | CF₃ | H | |
| 211 | Me | H | H | c-hexyl | H | 6-NO₂ | |
| 212 | Me | H | H | CH₂CH═CH₂ | CF₃ | H | |
| 213 | Me | H | H | CH═CHCH₃ | CN | H | |
| 214 | Me | H | H | CH═CH₂ | CF₃ | H | |
| 215 | Me | H | H | CH₂C≡CH | CF₃ | 5-CF₃ | |
| 216 | Me | H | H | C≡CCH₃ | Cl | 5-Cl | |
| 217 | Me | H | H | CH₂-c-Pr | CF₃ | H | |
| 218 | Me | H | H | CH₂-c-hexyl | H | 5-NO₂ | |
| 219 | Me | H | H | CF₃ | CF₃ | H | |
| 220 | Me | H | H | CHFCH₂CH₃ | CN | H | |
| 221 | Me | H | H | CHClCH₃ | CF₃ | H | |
| 222 | Me | H | H | CH₂OCH₃ | CF₃ | 3-CF₃ | |
| 223 | Me | H | H | CH₂OCH₂CH₃ | Cl | 3-Cl | |
| 224 | Me | H | H | CF(CH₃)₂ | CF₃ | H | |
| 225 | Me | H | H | 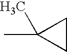 | H | 3-NO₂ | |
| 226 | Me | H | Me | H | CF₃ | H | |
| 227 | Me | H | Me | Me | CN | H | |
| 228 | Me | H | Me | Et | CF₃ | H | |
| 229 | Me | H | Me | Pr | CF₃ | 5-CF₃ | |
| 230 | Me | H | Me | i-Pr | Cl | 5-Cl | |
| 231 | Me | H | Me | c-Pr | CF₃ | H | |
| 232 | Me | H | Me | Bu | H | 5-NO₂ | |
| 233 | Me | H | Me | i-Bu | CF₃ | H | |
| 234 | Me | H | Me | c-Bu | CN | H | |
| 235 | Me | H | Me | t-Bu | CF₃ | H | |
| 236 | Me | H | Me | c-hexyl | CF₃ | 3-CF₃ | |
| 237 | Me | H | Me | CH₂CHCH₂ | Cl | 3-Cl | |
| 238 | Me | H | Me | CH═CHCH₃ | CF₃ | H | |
| 239 | Me | H | Me | CH═CH₂ | H | 3-NO₂ | |
| 240 | Me | H | Me | CH₂C≡CH | CF₃ | H | |
| 241 | Me | H | Me | C≡CCH₃ | CN | H | |
| 242 | Me | H | Me | CH₂-c-Pr | CF₃ | H | |
| 243 | Me | H | Me | CH₂-c-hexyl | CF₃ | 6-CF₃ | |
| 244 | Me | H | Me | CF₃ | Cl | 6-Cl | |
| 245 | Me | H | Me | CHFCH₂CH₃ | CF₃ | H | |
| 246 | Me | H | Me | CHClCH₃ | H | 6-NO₂ | |
| 247 | Me | H | Me | CH₂OCH₃ | CF₃ | H | |
| 248 | Me | H | Me | CH₂OCH₂CH₃ | CN | H | |
| 249 | Me | H | Me | CF(CH₃)₂ | CF₃ | H | |
| 250 | Me | H | Me | 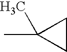 | CF₃ | 6-CF₃ | |
| 251 | Me | H | CO-Me | H | Cl | 6-Cl | |
| 252 | Me | H | CO-Me | Me | CF₃ | H | |
| 253 | Me | H | CO-Me | Et | H | 6-NO₂ | |
| 254 | Me | H | CO-Me | Pr | CF₃ | H | |
| 255 | Me | H | CO-Me | i-Pr | CN | H | |
| 256 | Me | H | CO-Me | c-Pr | CF₃ | H | |
| 257 | Me | H | CO-Me | Bu | CF₃ | 6-CF₃ | |
| 258 | Me | H | CO-Me | i-Bu | Cl | 6-Cl | |
| 259 | Me | H | CO-Me | c-Bu | CF₃ | H | |
| 260 | Me | H | CO-Me | t-Bu | H | 6-NO₂ | |
| 261 | Me | H | CO-Me | c-hexyl | CF₃ | H | |
| 262 | Me | H | CO-Me | CH₂CH═CH₂ | CN | H | |
| 263 | Me | H | CO-Me | CH═CHCH₃ | CF₃ | H | |
| 264 | Me | H | CO-Me | CH═CH₂ | CF₃ | 5-CF₃ | |
| 265 | Me | H | CO-Me | CH₂C≡CH | Cl | 5-Cl | |
| 266 | Me | H | CO-Me | C≡CCH₃ | CF₃ | H | |
| 267 | Me | H | CO-Me | CH₂-c-Pr | H | 5-NO₂ | |
| 268 | Me | H | CO-Me | CH₂-c-hexyl | CF₃ | H | |
| 269 | Me | H | CO-Me | CF₃ | CN | H | |
| 270 | Me | H | CO-Me | CHFCH₂CH₃ | CF₃ | H | |
| 271 | Me | H | CO-Me | CH | ClCH₃ | CF₃ | 3-CF₃ |
| 272 | Me | H | CO-Me | CH₂OCH₃ | Cl | 3-Cl | |
| 273 | Me | H | CO-Me | CH₂OCH₂CH₃ | CF₃ | H | |
| 274 | Me | H | CO-Me | CF(CH₃)₂ | H | 3-NO₂ | |
| 275 | Me | H | CO-Me | 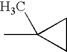 | CF₃ | H | |
| 276 | Me | H | CO-c-Pr | H | CN | H | |
| 277 | Me | H | CO-c-Pr | Me | CF₃ | H | |
| 278 | Me | H | CO-c-Pr | Et | CF₃ | 5-CF₃ | |
| 279 | Me | H | CO-c-Pr | Pr | Cl | 5-Cl | |
| 280 | Me | H | CO-c-Pr | i-Pr | CF₃ | H | |
| 281 | Me | H | CO-c-Pr | c-Pr | H | 5-NO₂ | |
| 282 | Me | H | CO-c-Pr | Bu | CF₃ | H | |
| 283 | Me | H | CO-c-Pr | i-Bu | CN | H | |
| 284 | Me | H | CO-c-Pr | c-Bu | CF₃ | H | |
| 285 | Me | H | CO-c-Pr | t-Bu | CF₃ | 3-CF₃ | |
| 286 | Me | H | CO-c-Pr | c-hexyl Cl | 3-Cl | | |
| 287 | Me | H | CO-c-Pr | CH₂CH═CH₂ | CF₃ | H | |
| 288 | Me | H | CO-c-Pr | CH═CHCH₃ | H | 3-NO₂ | |
| 289 | Me | H | CO-c-Pr | CH═CH₂ | CF₃ | H | |
| 290 | Me | H | CO-c-Pr | CH₂C≡CH | CN | H | |
| 291 | Me | H | CO-c-Pr | C≡CCH₃ | CF₃ | H | |
| 292 | Me | H | CO-c-Pr | CH₂-c-Pr | CF₃ | 5-CF₃ | |
| 293 | Me | H | CO-c-Pr | CH₂-c-hexyl | Cl | 5-Cl | |
| 294 | Me | H | CO-c-Pr | CF₃ | CF₃ | H | |
| 295 | Me | H | CO-c-Pr | CHFCH₂CH₃ | H | 5-NO₂ | |
| 296 | Me | H | CO-c-Pr | CHClCH₃ | CF₃ | H | |
| 297 | Me | H | CO-c-Pr | CH₂OCH₃ | CN | H | |
| 298 | Me | H | CO-c-Pr | CH₂OCH₂CH₃ | CF₃ | H | |
| 299 | Me | H | CO-c-Pr | CF(CH₃)₂ | CF₃ | 3-CF₃ | |
| 300 | Me | H | CO-c-Pr | 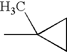 | Cl | 3-Cl | |
| 301 | Et | H | H | H | CF₃ | H | |
| 302 | Et | H | H | Me | H | 3-NO₂ | |
| 303 | Et | H | H | Et | CF₃ | H | |
| 304 | Et | H | H | Pr | CN | H | |
| 305 | Et | H | H | i-Pr | CF₃ | H | 6.79(s, 5-H, pyrimidine) |
| 306 | Et | H | H | c-Pr | CF₃ | H | 0.75(m, 2H), 0.95(m, 2H), 1.35(t, |
| | | | | | | | 3H), 1.42(m, 1H), 1.85(q, 2H), |
| | | | | | | | 4.55(d, 2H), 6.63(bs, 1H), 6.80 |
| | | | | | | | (s, 1H), 7.40(dd, 1H), 7.60(d, |
| | | | | | | | 1H), 8.75(d, 1H) |
| 307 | Et | H | H | c-Pr | Cl | H | 6.75(s, 5-H, pyrimidine) |
| 308 | Et | H | H | i-Bu | Cl | H | 6.70(s, 5-H, pyrimidine) |
| 309 | Et | H | H | t-Bu | CF₃ | H | 6.78(s, 5-H, pyrimidine) |
| 310 | Et | H | H | t-Bu | CF₃ | H | |
| 311 | Et | H | H | c-hexyl | CN | H | |
| 312 | Et | H | H | c-Pr | OCF₂H | H | 6.72(s, 5-H, pyrimidine) |
| 313 | Et | H | H | CH═CHCH₃ | CF₃ | 6-CF₃ | |
| 314 | Et | H | H | CH═CH₂ | Cl | 6-Cl | |
| 315 | Et | H | H | CH₂C≡CH | CF₃ | H | |
| 316 | Et | H | H | C≡CCH₃ | H | 6-NO₂ | |
| 317 | Et | H | H | CH₂-c-Pr | CF₃ | H | |
| 318 | Et | H | H | CH₂-c-hexyl | CN | H | |
| 319 | Et | H | H | CF₃ | CF₃ | H | 6.81(s, 5-H, pyrimidine) |
| 320 | Et | H | H | CHFCH₂CH₃ | CF₃ | 6-CF₃ | |
| 321 | Et | H | H | CHClCH₃ | CF₃ | H | 6.70(s, 5-H, pyrimidine) |
| 322 | Et | H | H | CH₂OCH₃ | CF₃ | H | |
| 323 | Et | H | H | CH₂OCH₂CH₃ | H | 6-NO₂ | |
| 324 | Et | H | H | CF(CH₃)₂ | CF₃ | H | 6.76(s, 5-H, pyrimidine) |
| 325 | Et | H | H | 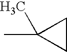 | Cl | H | 6.72(s, 5-H, pyrimidine) |
| 326 | Et | H | H | 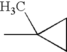 | CF₃ | H | 6.82(s, 5-H, pyrimidine) |
| 327 | Et | H | H | 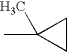 | OCF₂H | H | 6.80(s, 5-H, pyrimidine) |
| 328 | Et | H | Me | Et | Cl | 6-Cl | |
| 329 | H | Et | H | i-Pr | CF₃ | H | 8.50(S. 6-H, pyrimidine) |
| 330 | H | Et | H | i-Pr | OCF₂H | H | 8.48(s, 6-H, pyrimidine) |
| 331 | H | Et | H | c-Pr | CF₃ | H | |
| 332 | Et | H | Me | Bu | CN | H | |
| 333 | Et | H | Me | i-Bu | CF₃ | H | |
| 334 | Et | H | Me | c-Bu | CF₃ | 5-CF₃ | |
| 335 | Et | H | Me | t-Bu | Cl | 5-Cl | |
| 336 | Et | H | Me | c-hexyl | CF₃ | H | |
| 337 | (CH₂)₃ | | H | c-Pr | CF₃ | H | |
| 338 | OMe | H | H | c-Bu | OCHF₂ | H | 6.18(s, 5-H, pyrimidine) |
| 339 | OMe | H | H | c-Bu | CF₃ | H | 6.22(s, 5-H, pyrimidine) |
| 340 | OMe | H | H | c-Pr | OCHF₂ | H | 6.19(s, 5-H, pyrimidine) |
| 341 | OMe | H | H | c-Pr | CF₃ | H | 6.22(s, 5-H, pyrimidine) |
| 342 | OMe | H | H | i-Bu | OCHF₂ | H | 6.18(s, 5-H, pyrimidine) |
| 343 | OMe | H | H | i-Bu | CF₃ | H | 6.20(s, 5-H, pyrimidine) |
| 344 | OMe | H | H | Et | CF₃ | H | 6.20(s, 5-H, pyrimidine) |

TABLE 4
Compounds of the formula (I) according to the invention in which A is
A4 and X¹, X² are each hydrogen

| | |
|---|---|
| | (I) |
| 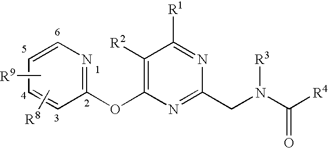 | |

| No. | R¹ | R² | R³ | R⁴ | R⁸ | R⁹ | ¹H-NMR: δ[CDCl₃] |
|---|---|---|---|---|---|---|---|
| 1 | H | H | H | H | H | 6-NO₂ | |
| 2 | H | H | H | Me | 4-CF₃ | H | |
| 3 | H | H | H | Et | 3-CN | H | |
| 4 | H | H | H | Pr | 5-CF₃ | H | |
| 5 | H | H | H | i-Pr | 3-CF₃ | 6-CF₃ | |
| 6 | H | H | H | c-Pr | 4-Cl | 6-Cl | |
| 7 | H | H | H | Bu | 5-CF₃ | H | |
| 8 | H | H | H | i-Bu | H | 6-NO₂ | |
| 9 | H | H | H | c-Bu | 4-CF₃ | H | |
| 10 | H | H | H | t-Bu | 3-CN | H | |
| 11 | H | H | H | c-hexyl | 5-CF₃ | H | |
| 12 | H | H | H | CH₂CH═CH₂ | 3-CF₃ | 6-CF₃ | |
| 13 | H | H | H | CH═CHCH₃ | 4-Cl | 6-Cl | |
| 14 | H | H | H | CH═CH₂ | 5-CF₃ | H | |
| 15 | H | H | H | CH₂C≡CH | H | 6-NO₂ | |
| 16 | H | H | H | C≡CCH₃ | 4-CF₃ | H | |
| 17 | H | H | H | CH₂-c-Pr | 3-CN | H | |
| 18 | H | H | H | CH₂-c-hexyl | 5-CF₃ | H | |
| 19 | H | H | H | CF₃ | 3-CF₃ | H | |
| 20 | H | H | H | CHFCH₂CH₃ | 4-Cl | 6-Cl | |
| 21 | H | H | H | CHClCH₃ | 5-CF₃ | H | |
| 22 | H | H | H | CH₂OCH₃ | H | 6-NO₂ | |
| 23 | H | H | H | CH₂OCH₂CH₃ | 4-CF₃ | H | |
| 24 | H | H | H | CF(CH₃)₂ | 3-CN | H | |
| 25 | H | H | H | 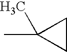 | 5-CF₃ | H | |
| 26 | H | H | Me | H | 3-CF₃ | 6-CF₃ | |
| 27 | H | H | Me | Me | 4-Cl | 6-Cl | |
| 28 | H | H | Me | Et | 5-CF₃ | H | |
| 29 | H | H | Me | Pr | H | 6-NO₂ | |
| 30 | H | H | Me | i-Pr | 4-CF₃ | H | |
| 31 | H | H | Me | c-Pr | 3-CN | H | |
| 32 | H | H | Me | Bu | 5-CF₃ | H | |
| 33 | H | H | Me | i-Bu | 3-CF₃ | 6-CF₃ | |
| 34 | H | H | Me | c-Bu | 4-Cl | 6-Cl | |
| 35 | H | H | Me | t-Bu | 5-CF₃ | H | |
| 36 | H | H | Me | c-hexyl | H | 6-NO₂ | |
| 37 | H | H | Me | CH₂CH═CH₂ | 4-CF₃ | H | |
| 38 | H | H | Me | CH═CHCH₃ | 3-CN | H | |
| 39 | H | H | Me | CH═CH₂ | 5-CF₃ | H | |
| 40 | H | H | Me | CH₂C≡CH | 3-CF₃ | 6-CF₃ | |
| 41 | H | H | Me | C≡CCH₃ | 4-Cl | 6-Cl | |
| 42 | H | H | Me | CH₂-c-Pr | 5-CF₃ | H | |
| 43 | H | H | Me | CH₂-c-hexyl | H | 6-NO₂ | |
| 44 | H | H | Me | CF₃ | 4-CF₃ | H | |
| 45 | H | H | Me | CHFCH₂CH₃ | 3-CN | H | |
| 46 | H | H | Me | CHClCH₃ | 5-CF₃ | H | |
| 47 | H | H | Me | CH₂OCH₃ | 3-CF₃ | 6-CF₃ | |
| 48 | H | H | Me | CH₂OCH₂CH₃ | 4-Cl | 6-Cl | |
| 49 | H | H | Me | CF(CH₃)₂ | 5-CF₃ | H | |
| 50 | H | H | Me | 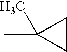 | H | 6-NO₂ | |
| 51 | H | H | CO-Me | H | 4-CF₃ | H | |
| 52 | H | H | CO-Me | Me | 3-CN | H | |
| 53 | H | H | CO-Me | Et | 5-CF₃ | H | |
| 54 | H | H | CO-Me | Pr | 3-CF₃ | 6-CF₃ | |
| 55 | H | H | CO-Me | i-Pr | 4-Cl | 6-Cl | |
| 56 | H | H | CO-Me | c-Pr | 5-CF₃ | H | |
| 57 | H | H | CO-Me | Bu | H | 6-NO₂ | |
| 58 | H | H | CO-Me | i-Bu | 4-CF₃ | H | |
| 59 | H | H | CO-Me | c-Bu | 3-CN | H | |
| 60 | H | H | CO-Me | t-Bu | 5-CF₃ | H | |
| 61 | H | H | CO-Me | c-hexyl | 3-CF₃ | 6-CF₃ | |
| 62 | H | H | CO-Me | OH₂CH═CH₂ | 4-Cl | 6-Cl | |
| 63 | H | H | CO-Me | CH═CHCH₃ | 5-CF₃ | H | |
| 64 | H | H | CO-Me | CH═CH₂ | H | 6-NO₂ | |
| 65 | H | H | CO-Me | CH₂C≡CH | 4-CF₃ | H | |
| 66 | H | H | CO-Me | C≡CCH₃ | 3-CN | H | |
| 67 | H | H | CO-Me | CH₂-c-Pr | 5-CF₃ | H | |
| 68 | H | H | CO-Me | CH₂-c-hexyl | 3-CF₃ | 6-CF₃ | |
| 69 | H | H | CO-Me | CF₃ | 4-Cl | 6-Cl | |
| 70 | H | H | CO-Me | CHFCH₂CH₃ | 5-CF₃ | H | |
| 71 | H | H | CO-Me | CHClCH₃ | H | 6-NO₂ | |
| 72 | H | H | CO-Me | CH₂OCH₃ | 4-CF₃ | H | |
| 73 | H | H | CO-Me | CH₂OCH₂CH₃ | 3-CN | H | |
| 74 | H | H | CO-Me | CF(CH₃)₂ | 5-CF₃ | H | |
| 75 | H | H | CO-Me | 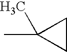 | 3-CF₃ | 6-CF₃ | |
| 76 | H | H | CO-c-Pr | H | 4-Cl | 6-Cl | |
| 77 | H | H | CO-c-Pr | Me | 5-CF₃ | H | |
| 78 | H | H | CO-c-Pr | Et | H | 6-NO₂ | |
| 79 | H | H | CO-c-Pr | Pr | 3-CF₃ | H | |
| 80 | H | H | CO-c-Pr | i-Pr | 4-CN | H | |
| 81 | H | H | CO-c-Pr | c-Pr | 4-CF₃ | H | |
| 82 | H | H | CO-c-Pr | Bu | 5-CF₃ | 6-CF₃ | |
| 83 | H | H | CO-c-Pr | i-Bu | 4-Cl | 6-Cl | |
| 84 | H | H | CO-c-Pr | c-Bu | 5-CF₃ | H | |
| 85 | H | H | CO-c-Pr | t-Bu | H | 6-NO₂ | |
| 86 | H | H | CO-c-Pr | c-hexyl | 4-CF₃ | H | |
| 87 | H | H | CO-c-Pr | CH₂CH═CH₂ | 3-CN | H | |
| 88 | H | H | CO-c-Pr | CH═CHCH₃ | 5-CF₃ | H | |
| 89 | H | H | CO-c-Pr | CH═CH₂ | 3-CF₃ | 6-CF₃ | |
| 90 | H | H | CO-c-Pr | CH₂C≡CH | 4-Cl | 6-Cl | |
| 91 | H | H | CO-c-Pr | C≡CCH₃ | 5-CF₃ | H | |
| 92 | H | H | CO-c-Pr | CH₂-c-Pr | H | 6-NO₂ | |
| 93 | H | H | CO-c-Pr | CH₂-c-hexyl | 4-CF₃ | H | |
| 94 | H | H | CO-c-Pr | CF₃ | 3-CN | H | |
| 95 | H | H | CO-c-Pr | CHFCH₂CH₃ | 5-CF₃ | H | |
| 96 | H | H | CO-c-Pr | CHClCH₃ | 3-CF₃ | 6-CF₃ | |
| 97 | H | H | CO-c-Pr | CH₂OCH₃ | 4-Cl | 6-Cl | |
| 98 | H | H | CO-c-Pr | CH₂OCH₂CH₃ | 5-CF₃ | H | |
| 99 | H | H | CO-c-Pr | CF(CH₃)₂ | H | 6-NO₂ | |
| 100 | H | H | CO-c-Pr | 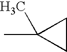 | 4-CF₃ | H | |
| 101 | Cl | H | H | H | 3-CN | H | |
| 102 | Cl | H | H | Me | 5-CF₃ | H | |
| 103 | Cl | H | H | Et | 3-CF₃ | 6-CF₃ | |
| 104 | Cl | H | H | Pr | 4-Cl | 6-Cl | |
| 105 | Cl | H | H | i-Pr | 5-CF₃ | H | |
| 106 | Cl | H | H | c-Pr | H | 6-NO₂ | |
| 107 | Cl | H | H | Bu | 3-CF₃ | H | |
| 108 | Cl | H | H | i-Bu | 4-CN | H | |
| 109 | Cl | H | H | c-Bu | 4-CF₃ | H | |
| 110 | Cl | H | H | t-Bu | 5-CF₃ | 6-CF₃ | |
| 111 | Cl | H | H | c-hexyl | 4-Cl | 6-Cl | |
| 112 | Cl | H | H | CH₂CH═CH₂ | 5-CF₃ | H | |
| 113 | Cl | H | H | CH═CHCH₃ | H | 6-NO₂ | |
| 114 | Cl | H | H | CH═CH₂ | 4-CF₃ | H | |
| 115 | Cl | H | H | CH₂C≡CH | 3-CN | H | |
| 116 | Cl | H | H | C≡CCH₃ | 5-CF₃ | H | |
| 117 | Cl | H | H | CH₂-c-Pr | 3-CF₃ | 6-CF₃ | |
| 118 | Cl | H | H | CH₂-c-hexyl | 4-Cl | 6-Cl | |
| 119 | Cl | H | H | CF₃ | 5-CF₃ | H | |
| 120 | Cl | H | H | CHFCH₂CH₃ | H | 6-NO₂ | |
| 121 | Cl | H | H | CHClCH₃ | 4-CF₃ | H | |
| 122 | Cl | H | H | CH₂OCH₃ | 3-CN | H | |
| 123 | Cl | H | H | CH₂OCH₂CH₃ | 5-CF₃ | H | |
| 124 | Cl | H | H | CF(CH₃)₂ | 3-CF₃ | 6-CF₃ | |
| 125 | Cl | H | H | 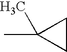 | 4-Cl | 6-Cl | |
| 126 | Cl | H | Me | H | 5-CF₃ | H | |
| 127 | Cl | H | Me | Me | H | 6-NO₂ | |
| 128 | Cl | H | Me | Et | 4-CF₃ | H | |
| 129 | Cl | H | Me | Pr | 3-CN | H | |
| 130 | Cl | H | Me | i-Pr | 5-CF₃ | H | |
| 131 | Cl | H | Me | c-Pr | 3-CF₃ | 6-CF₃ | |
| 132 | Cl | H | Me | Bu | 4-Cl | 6-Cl | |
| 133 | Cl | H | Me | i-Bu | 5-CF₃ | H | |
| 134 | Cl | H | Me | c-Bu | H | 6-NO₂ | |
| 135 | Cl | H | Me | t-Bu | 3-CF₃ | H | |
| 136 | Cl | H | Me | c-hexyl | 4-CN | H | |
| 137 | Cl | H | Me | CH₂CH═CH₂ | 4-CF₃ | H | |
| 138 | Cl | H | Me | CH═CHCH₃ | 5-CF₃ | 6-CF₃ | |
| 139 | Cl | H | Me | CH═CH₂ | 4-Cl | 6-Cl | |
| 140 | Cl | H | Me | CH₂C≡CH | 5-CF₃ | H | |
| 141 | Cl | H | Me | C≡CCH₃ | H | 6-NO₂ | |
| 142 | Cl | H | Me | CH₂-c-Pr | 4-CF₃ | H | |
| 143 | Cl | H | Me | CH₂-c-hexyl | 3-CN | H | |
| 144 | Cl | H | Me | CF₃ | 5-CF₃ | H | |
| 145 | Cl | H | Me | CHFCH₂CH₃ | 3-CF₃ | 6-CF₃ | |
| 146 | Cl | H | Me | CHClCH₃ | 4-Cl | 6-Cl | |
| 147 | Cl | H | Me | CH₂OCH₃ | 5-CF₃ | H | |
| 148 | Cl | H | Me | CH₂OCH₂CH₃ | H | 6-NO₂ | |
| 149 | Cl | H | Me | CF(CH₃)₂ | 4-CF₃ | H | |
| 150 | Cl | H | Me | 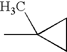 | 3-CN | H | |
| 151 | Cl | H | CO-Me | H | 5-CF₃ | H | |
| 152 | Cl | H | CO-Me | Me | 3-CF₃ | 6-CF₃ | |
| 153 | Cl | H | CO-Me | Et | 4-Cl | 6-Cl | |
| 154 | Cl | H | CO-Me | Pr | 5-CF₃ | H | |
| 155 | Cl | H | CO-Me | i-Pr | H | 6-NO₂ | |
| 156 | Cl | H | CO-Me | c-Pr | 4-CF₃ | H | |
| 157 | Cl | H | CO-Me | Bu | 3-CN | H | |
| 158 | Cl | H | CO-Me | i-Bu | 5-CF₃ | H | |
| 159 | Cl | H | CO-Me | c-Bu | 3-CF₃ | 6-CF₃ | |
| 160 | Cl | H | CO-Me | t-Bu | 4-Cl | 6-Cl | |
| 161 | Cl | H | CO-Me | c-hexyl | 5-CF₃ | H | |
| 162 | Cl | H | CO-Me | CH₂CH═CH₂ | H | 6-NO₂ | |
| 163 | Cl | H | CO-Me | CH═CHCH₃ | 3-CF₃ | H | |
| 164 | Cl | H | CO-Me | CH═CH₂ | 4-CN | H | |
| 165 | Cl | H | CO-Me | CH₂C≡CH | 4-CF₃ | H | |
| 166 | Cl | H | CO-Me | C≡CCH₃ | 5-CF₃ | 6-CF₃ | |
| 167 | Cl | H | CO-Me | CH₂-c-Pr | 4-Cl | 6-Cl | |
| 168 | Cl | H | CO-Me | CH₂-c-hexyl | 5-CF₃ | H | |
| 169 | Cl | H | CO-Me | CF₃ | H | 6-NO₂ | |
| 170 | Cl | H | CO-Me | CHFCH₂CH₃ | 4-CF₃ | H | |
| 171 | Cl | H | CO-Me | CHClCH₃ | 3-CN | H | |
| 172 | Cl | H | CO-Me | CH₂OCH₃ | 5-CF₃ | H | |
| 173 | Cl | H | CO-Me | CH₂OCH₂CH₃ | 3-CF₃ | 6-CF₃ | |
| 174 | Cl | H | CO-Me | CF(CH₃)₂ | 4-Cl | 6-Cl | |
| 175 | Cl | H | CO-Me | 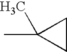 | 5-CF₃ | H | |
| 176 | Cl | H | CO-c-Pr | H | H | 6-NO₂ | |
| 177 | Cl | H | CO-c-Pr | Me | 4-CF₃ | H | |
| 178 | Cl | H | CO-c-Pr | Et | 3-CN | H | |
| 179 | Cl | H | CO-c-Pr | Pr | 5-CF₃ | H | |
| 180 | Cl | H | CO-c-Pr | i-Pr | 3-CF₃ | 6-CF₃ | |
| 181 | Cl | H | CO-c-Pr | c-Pr | 4-Cl | 6-Cl | |
| 182 | Cl | H | CO-c-Pr | Bu | 5-CF₃ | H | |
| 183 | Cl | H | CO-c-Pr | i-Bu | H | 6-NO₂ | |
| 184 | Cl | H | CO-c-Pr | c-Bu | 4-CF₃ | H | |
| 185 | Cl | H | CO-c-Pr | t-Bu | 3-CN | H | |
| 186 | Cl | H | CO-c-Pr | c-hexyl 5-CF₃ | H | | |
| 187 | Cl | H | CO-c-Pr | CH₂CH═CH₂ | 3-CF₃ | 6-CF₃ | |
| 188 | Cl | H | CO-c-Pr | CH═CHCH₃ | 4-Cl | 6-Cl | |
| 189 | Cl | H | CO-c-Pr | CH═CH₂ | 5-CF₃ | H | |
| 190 | Cl | H | CO-c-Pr | CH₂C≡CH | H | 6-NO₂ | |
| 191 | Cl | H | CO-c-Pr | C≡CCH₃ | 3-CF₃ | H | |
| 192 | Cl | H | CO-c-Pr | CH₂-c-Pr | 4-CN | H | |
| 193 | Cl | H | CO-c-Pr | CH₂-c-hexyl | 4-CF₃ | H | |
| 194 | Cl | H | CO-c-Pr | CF₃ | 5-CF₃ | 6-CF₃ | |
| 195 | Cl | H | CO-c-Pr | CHFCH₂CH₃ | 4-Cl | 6-Cl | |
| 196 | Cl | H | CO-c-Pr | CHClCH₃ | 5-CF₃ | H | |
| 197 | Cl | H | CO-c-Pr | CH₂OCH₃ | H | 6-NO₂ | |
| 198 | Cl | H | CO-c-Pr | CH₂OCH₂CH₃ | 4-CF₃ | H | |
| 199 | Cl | H | CO-c-Pr | CF(CH₃)₂ | 3-CN | H | |
| 200 | Cl | H | CO-c-Pr | 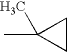 | 5-CF₃ | H | |
| 201 | Me | H | H | H | 3-CF₃ | 6-CF₃ | |
| 202 | Me | H | H | Me | 4-Cl | 6-Cl | |
| 203 | Me | H | H | Et | 5-CF₃ | H | |
| 204 | Me | H | H | Pr | H | 6-NO₂ | |
| 205 | Me | H | H | i-Pr | 6-CN | H | |
| 206 | Me | H | H | c-Pr | H | 6-CF₃ | 6.86(s, 5-H, pyrimidine) |
| 207 | Me | H | H | Bu | 3-CF₃ | H | |
| 208 | Me | H | H | i-Bu | 4-CF₃ | 6-CF₃ | |
| 209 | Me | H | H | c-Bu | 6-Cl | 6-Cl | |
| 210 | Me | H | H | t-Bu | 5-CF₃ | H | |
| 211 | Me | H | H | c-hexyl | H | 6-NO₂ | |
| 212 | Me | H | H | CH₂CH═CH₂ | 4-CF₃ | H | |
| 213 | Me | H | H | CH═CHCH₃ | 3-CN | H | |
| 214 | Me | H | H | CH═CH₂ | 5-CF₃ | H | |
| 215 | Me | H | H | CH₂C≡CH | 3-CF₃ | 6-CF₃ | |
| 216 | Me | H | H | C≡CCH₃ | 4-Cl | 6-Cl | |
| 217 | Me | H | H | CH₂-c-Pr | 5-CF₃ | H | |
| 218 | Me | H | H | CH₂-c-hexyl | H | 6-NO₂ | |
| 219 | Me | H | H | CF₃ | 4-CF₃ | H | |
| 220 | Me | H | H | CHFCH₂CH₃ | 3-CN | H | |
| 221 | Me | H | H | CHClCH₃ | 5-CF₃ | H | |
| 222 | Me | H | H | CH₂OCH₃ | 3-CF₃ | 6-CF₃ | |
| 223 | Me | H | H | CH₂OCH₂CH₃ | 4-Cl | 6-Cl | |
| 224 | Me | H | H | CF(CH₃)₂ | 5-CF₃ | H | |
| 225 | Me | H | H | 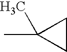 | H | 6-NO₂ | |
| 226 | Me | H | Me | H | 4-CF₃ | H | |
| 227 | Me | H | Me | Me | 3-CN | H | |
| 228 | Me | H | Me | Et | 5-CF₃ | H | |
| 229 | Me | H | Me | Pr | 3-CF₃ | 6-CF₃ | |
| 230 | Me | H | Me | i-Pr | 4-Cl | 6-Cl | |
| 231 | Me | H | Me | c-Pr | 5-CF₃ | H | |
| 232 | Me | H | Me | Bu | H | 6-NO₂ | |
| 233 | Me | H | Me | i-Bu | 4-CF₃ | H | |
| 234 | Me | H | Me | c-Bu | 3-CN | H | |
| 235 | Me | H | Me | t-Bu | 5-CF₃ | H | |
| 236 | Me | H | Me | c-hexyl | 3-CF₃ | 6-CF₃ | |
| 237 | Me | H | Me | CH₂CH═CH₂ | 4-Cl | 6-Cl | |
| 238 | Me | H | Me | CH═CHCH₃ | 5-CF₃ | H | |
| 239 | Me | H | Me | CH═CH₂ | H | 6-NO₂ | |
| 240 | Me | H | Me | CH₂C≡CH | 4-CF₃ | H | |
| 241 | Me | H | Me | C≡CCH₃ | 3-CN | H | |
| 242 | Me | H | Me | CH₂-c-Pr | 5-CF₃ | H | |
| 243 | Me | H | Me | CH₂-c-hexyl | 3-CF₃ | 6-CF₃ | |
| 244 | Me | H | Me | CF₃ | 4-Cl | 6-Cl | |
| 245 | Me | H | Me | CHFCH₂CH₃ | 5-CF₃ | H | |
| 246 | Me | H | Me | CHClCH₃ | H | 6-NO₂ | |
| 247 | Me | H | Me | CH₂OCH₃ | 4-CF₃ | H | |
| 248 | Me | H | Me | CH₂OCH₂CH₃ | 3-CN | H | |
| 249 | Me | H | Me | CF(CH₃)₂ | 5-CF₃ | H | |
| 250 | Me | H | Me | 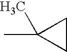 | 3-CF₃ | 6-CF₃ | |
| 251 | Me | H | CO-Me | H | 4-Cl | 6-Cl | |
| 252 | Me | H | CO-Me | Me | 5-CF₃ | H | |
| 253 | Me | H | CO-Me | Et | H | 6-NO₂ | |
| 254 | Me | H | CO-Me | Pr | 4-CF₃ | H | |
| 255 | Me | H | CO-Me | i-Pr | 3-CN | H | |
| 256 | Me | H | CO-Me | c-Pr | 5-CF₃ | H | |
| 257 | Me | H | CO-Me | Bu | 3-CF₃ | 6-CF₃ | |
| 258 | Me | H | CO-Me | i-Bu | 4-Cl | 6-Cl | |
| 259 | Me | H | CO-Me | c-Bu | 5-CF₃ | H | |
| 260 | Me | H | CO-Me | t-Bu | H | 6-NO₂ | |
| 261 | Me | H | CO-Me | c-hexyl | 4-CF₃ | H | |
| 262 | Me | H | CO-Me | CH₂CH═CH₂ | 3-CN | H | |
| 263 | Me | H | CO-Me | CH═CHCH₃ | 5-CF₃ | H | |
| 264 | Me | H | CO-Me | CH═CH₂ | 3-CF₃ | 6-CF₃ | |
| 265 | Me | H | CO-Me | CH₂C≡CH | 4-Cl | 6-Cl | |
| 266 | Me | H | CO-Me | C≡CCH₃ | 5-CF₃ | H | |
| 267 | Me | H | CO-Me | CH₂-c-Pr | H | 6-NO₂ | |
| 268 | Me | H | CO-Me | CH₂-c-hexyl | 4-CF₃ | H | |
| 269 | Me | H | CO-Me | CF₃ | 3-CN | H | |
| 270 | Me | H | CO-Me | CHFCH₂CH₃ | 5-CF₃ | H | |
| 271 | Me | H | CO-Me | CHClCH₃ | 3-CF₃ | 6-CF₃ | |
| 272 | Me | H | CO-Me | CH₂OCH₃ | 4-Cl | 6-Cl | |
| 273 | Me | H | CO-Me | CH₂OCH₂CH₃ | 5-CF₃ | H | |
| 274 | Me | H | CO-Me | CF(CH₃)₂ | H | 6-NO₂ | |
| 275 | Me | H | CO-Me | 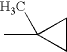 | 4-CF₃ | H | |
| 276 | Me | H | CO-c-Pr | H | 3-CN | H | |
| 277 | Me | H | CO-c-Pr | Me | 5-CF₃ | H | |
| 278 | Me | H | CO-c-Pr | Et | 3-CF₃ | 6-CF₃ | |
| 279 | Me | H | CO-c-Pr | Pr | 4-Cl | 6-Cl | |
| 280 | Me | H | CO-c-Pr | i-Pr | 5-CF₃ | H | |
| 281 | Me | H | CO-c-Pr | c-Pr | H | 6-NO₂ | |
| 282 | Me | H | CO-c-Pr | Bu | 4-CF₃ | H | |
| 283 | Me | H | CO-c-Pr | i-Bu | 3-CN | H | |
| 284 | Me | H | CO-c-Pr | c-Bu | 5-CF₃ | H | |
| 285 | Me | H | CO-c-Pr | t-Bu | 3-CF₃ | 6-CF₃ | |
| 286 | Me | H | CO-c-Pr | c-hexyl | 4-Cl | 6-Cl | |
| 287 | Me | H | CO-c-Pr | CH₂CH═CH₂ | 5-CF₃ | H | |
| 288 | Me | H | CO-c-Pr | CH═CHCH₃ | H | 6-NO₂ | |
| 289 | Me | H | CO-c-Pr | CH═CH₂ | 3-CF₃ | H | |
| 290 | Me | H | CO-c-Pr | CH₂C≡CH | 4-CN | H | |
| 291 | Me | H | CO-c-Pr | C≡CCH₃ | 4-CF₃ | H | |
| 292 | Me | H | CO-c-Pr | CH₂-c-Pr | 5-CF₃ | 6-CF₃ | |
| 293 | Me | H | CO-c-Pr | CH₂-c-hexyl | 4-Cl | 6-Cl | |
| 294 | Me | H | CO-c-Pr | CF₃ | 5-CF₃ | H | |
| 295 | Me | H | CO-c-Pr | CHFCH₂CH₃ | H | 6-NO₂ | |
| 296 | Me | H | CO-c-Pr | CHClCH₃ | 4-CF₃ | H | |
| 297 | Me | H | CO-c-Pr | CH₂OCH₃ | 3-CN | H | |
| 298 | Me | H | CO-c-Pr | CH₂OCH₂CH₃ | 5-CF₃ | H | |
| 299 | Me | H | CO-c-Pr | CF(CH₃)₂ | 3-CF₃ | 6-CF₃ | |
| 300 | Me | H | CO-c-Pr | 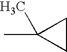 | 4-Cl | 6-Cl | |
| 301 | Et | H | H | H | 5-CF₃ | H | |
| 302 | Et | H | H | Me | H | 6-NO₂ | |
| 303 | Et | H | H | Et | 4-CF₃ | H | |
| 304 | Et | H | H | Pr | 3-CN | H | |
| 305 | Et | H | H | i-Pr | 5-CF₃ | H | |
| 306 | Et | H | H | c-Pr | 6-CF₃ | H | 6.94(s, 5-H, pyrimidine) |
| 307 | Et | H | H | Bu | 4-Cl | 6-Cl | |
| 308 | Et | H | H | i-Bu | 5-CF₃ | H | |
| 309 | Et | H | H | c-Bu | H | 6-NO₂ | |
| 310 | Et | H | H | t-Bu | 4-CF₃ | H | |
| 311 | (CH₂)₃ | H | c-Pr | CF₃ | H | | |

TABLE 5
Compounds of the formula (I) according to the invention in which A is
A5 and X¹, X² are each hydrogen

| | |
|---|---|
| | (I) |
| 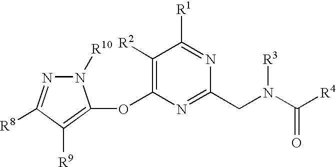 | |

| No. | R¹ | R² | R³ | R⁴ | R⁸ | R⁹ | R¹⁰ | ¹H-NMR: δ[CDCl₃] |
|---|---|---|---|---|---|---|---|---|
| 1 | H | H | H | H | H | CF₃ | Me | |
| 2 | H | H | H | Me | CF₃ | H | Me | |
| 3 | H | H | H | Et | CN | H | Me | |
| 4 | H | H | H | Pr | CF₃ | H | Me | |
| 5 | H | H | H | i-Pr | CF₃ | H | Me | |
| 6 | H | H | H | c-Pr | Cl | Cl | Me | |
| 7 | H | H | H | Bu | CF₃ | H | Me | |
| 8 | H | H | H | i-Bu | H | CF₃ | Me | |
| 9 | H | H | H | c-Bu | CF₃ | H | Me | |
| 10 | H | H | H | t-Bu | CN | H | Me | |
| 11 | H | H | H | c-hexyl | CF₃ | H | Me | |
| 12 | H | H | H | CH₂CH═CH₂ | CF₃ | H | Me | |
| 13 | H | H | H | CH═CHCH₃ | Cl | Cl | Me | |
| 14 | H | H | H | CH═CH₂ | CF₃ | H | Me | |
| 15 | H | H | H | CH₂C≡CH | H | CF₃ | Me | |
| 16 | H | H | H | C≡CCH₃ | CF₃ | H | Me | |
| 17 | H | H | H | CH₂-c-Pr | CN | H | Me | |
| 18 | H | H | H | CH₂-c-hexyl | CF₃ | H | Me | |
| 19 | H | H | H | CF₃ | CF₃ | H | Me | |
| 20 | H | H | H | CHFCH₂CH₃ | Cl | Cl | Me | |
| 21 | H | H | H | CHClCH₃ | CF₃ | H | Me | |
| 22 | H | H | H | CH₂OCH₃ | H | CF₃ | Me | |
| 23 | H | H | H | CH₂OCH₂CH₃ | CF₃ | H | Me | |
| 24 | H | H | H | CF(CH₃)₂ | CN | H | Me | |
| 25 | H | H | H | 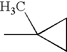 | CF₃ | H | Me | |
| 26 | H | H | Me | H | CF₃ | H | Me | |
| 27 | H | H | Me | Me | Cl | Cl | Me | |
| 28 | H | H | Me | Et | CF₃ | H | Me | |
| 29 | H | H | Me | Pr | H | CF₃ | Me | |
| 30 | H | H | Me | i-Pr | CF₃ | H | Me | |
| 31 | H | H | Me | c-Pr | CN | H | Me | |
| 32 | H | H | Me | Bu | CF₃ | H | Me | |
| 33 | H | H | Me | i-Bu | CF₃ | H | Me | |
| 34 | H | H | Me | c-Bu | Cl | Cl | Me | |
| 35 | H | H | Me | t-Bu | CF₃ | H | Me | |
| 36 | H | H | Me | c-hexyl | H | CF₃ | Me | |
| 37 | H | H | Me | CH₂CH═CH₂ | CF₃ | H | Me | |
| 38 | H | H | Me | CH═CHCH₃ | CN | H | Me | |
| 39 | H | H | Me | CH═CH₂ | CF₃ | H | Me | |
| 40 | H | H | Me | CH₂C≡CH | CF₃ | CF₃ | Me | |
| 41 | H | H | Me | C≡CCH₃ | Cl | H | Me | |
| 42 | H | H | Me | CH₂-c-Pr | CF₃ | H | Me | |
| 43 | H | H | Me | CH₂-c-hexyl | H | CF₃ | Me | |
| 44 | H | H | Me | CF₃ | CF₃ | H | Me | |
| 45 | H | H | Me | CHFCH₂CH₃ | CN | H | Me | |
| 46 | H | H | Me | CHClCH₃ | CF₃ | H | Me | |
| 47 | H | H | Me | CH₂OCH₃ | CF₃ | H | Me | |
| 48 | H | H | Me | CH₂OCH₂CH₃ | Cl | H | Me | |
| 49 | H | H | Me | CF(CH₃)₂ | CF₃ | H | Me | |
| 50 | H | H | Me | 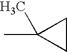 | H | CF₃ | Me | |
| 51 | H | H | CO-Me | H | CF₃ | H | Me | |
| 52 | H | H | CO-Me | Me | CN | H | Me | |
| 53 | H | H | CO-Me | Et | CF₃ | H | Me | |
| 54 | H | H | CO-Me | Pr | CF₃ | H | Me | |
| 55 | H | H | CO-Me | i-Pr | Cl | H | Me | |
| 56 | H | H | CO-Me | c-Pr | CF₃ | H | Me | |
| 57 | H | H | CO-Me | Bu | H | CF₃ | Me | |
| 58 | H | H | CO-Me | i-Bu | CF₃ | H | Me | |
| 59 | H | H | CO-Me | c-Bu | CN | H | Me | |
| 60 | H | H | CO-Me | t-Bu | CF₃ | H | Me | |
| 61 | H | H | CO-Me | c-hexyl | CF₃ | H | Me | |
| 62 | H | H | CO-Me | CH₂CH═CH₂ | Cl | H | Me | |
| 63 | H | H | CO-Me | CH═CHCH₃ | CF₃ | H | Me | |
| 64 | H | H | CO-Me | CH═CH₂ | H | CF₃ | Me | |
| 65 | H | H | CO-Me | CH₂C≡CH | CF₃ | H | Me | |
| 66 | H | H | CO-Me | C≡CCH₃ | CN | H | Me | |
| 67 | H | H | CO-Me | CH₂-c-Pr | CF₃ | H | Me | |
| 68 | H | H | CO-Me | CH₂-c-hexyl | CF₃ | H | Me | |
| 69 | H | H | CO-Me | CF₃ | Cl | H | Me | |
| 70 | H | H | CO-Me | CHFCH₂CH₃ | CF₃ | H | Me | |
| 71 | H | H | CO-Me | CHClCH₃ | H | CF₃ | Me | |
| 72 | H | H | CO-Me | CH₂OCH₃ | CF₃ | H | Me | |
| 73 | H | H | CO-Me | CH₂OCH₂CH₃ | CN | H | Me | |
| 74 | H | H | CO-Me | CF(CH₃)₂ | CF₃ | H | Me | |
| 75 | H | H | CO-Me | 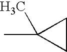 | CF₃ | H | Me | |
| 76 | H | H | CO-c-Pr | H | Cl | H | Me | |
| 77 | H | H | CO-c-Pr | Me | CF₃ | H | Me | |
| 78 | H | H | CO-c-Pr | Et | H | CF₃ | Me | |
| 79 | H | H | CO-c-Pr | Pr | CF₃ | H | Me | |
| 80 | H | H | CO-c-Pr | i-Pr | CN | H | Me | |
| 81 | H | H | CO-c-Pr | c-Pr | CF₃ | H | Me | |
| 82 | H | H | CO-c-Pr | Bu | CF₃ | H | Me | |
| 83 | H | H | CO-c-Pr | i-Bu | Cl | H | Me | |
| 84 | H | H | CO-c-Pr | c-Bu | CF₃ | H | Me | |
| 85 | H | H | CO-c-Pr | t-Bu | H | CF₃ | Me | |
| 86 | H | H | CO-c-Pr | c-hexyl | CF₃ | H | Me | |
| 87 | H | H | CO-c-Pr | CH₂CH═CH₂ | CN | H | Me | |
| 88 | H | H | CO-c-Pr | CH═CHCH₃ | CF₃ | H | Me | |
| 89 | H | H | CO-c-Pr | CH═CH₂ | CF₃ | H | Me | |
| 90 | H | H | CO-c-Pr | CH₂C≡CH | Cl | H | Me | |
| 91 | H | H | CO-c-Pr | C≡CCH₃ | CF₃ | H | Me | |
| 92 | H | H | CO-c-Pr | CH₂-c-Pr | H | CF₃ | Me | |
| 93 | H | H | CO-c-Pr | CH₂-c-hexyl | CF₃ | H | Me | |
| 94 | H | H | CO-c-Pr | CF₃ | CN | H | Me | |
| 95 | H | H | CO-c-Pr | CHFCH₂CH₃ | CF₃ | H | Me | |
| 96 | H | H | CO-c-Pr | CHClCH₃ | CF₃ | H | Me | |
| 97 | H | H | CO-c-Pr | CH₂OCH₃ | Cl | H | Me | |
| 98 | H | H | CO-c-Pr | CH₂OCH₂CH₃ | CF₃ | H | Me | |
| 99 | H | H | CO-c-Pr | CF(CH₃)₂ | H | CF₃ | Me | |
| 100 | H | H | CO-c-Pr | 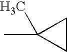 | CF₃ | H | Me | |
| 101 | Cl | H | H | H | CN | H | Me | |
| 102 | Cl | H | H | Me | CF₃ | H | Me | |
| 103 | Cl | H | H | Et | CF₃ | H | Me | |
| 104 | Cl | H | H | Pr | Cl | H | Me | |
| 105 | Cl | H | H | i-Pr | CF₃ | H | Me | |
| 106 | Cl | H | H | c-Pr | H | CF₃ | Me | |
| 107 | Cl | H | H | Bu | CF₃ | H | Me | |
| 108 | Cl | H | H | i-Bu | CN | H | Me | |
| 109 | Cl | H | H | c-Bu | CF₃ | H | Me | |
| 110 | Cl | H | H | t-Bu | CF₃ | H | Me | |
| 111 | Cl | H | H | c-hexyl | Cl | H | Me | |
| 112 | Cl | H | H | CH₂CH═CH₂ | CF₃ | H | Me | |
| 113 | Cl | H | H | CH═CHCH₃ | H | CF₃ | Me | |
| 114 | Cl | H | H | CH═CH₂ | CF₃ | H | Me | |
| 115 | Cl | H | H | CH₂C≡CH | CN | H | Me | |
| 116 | Cl | H | H | C≡CCH₃ | CF₃ | H | Me | |
| 117 | Cl | H | H | CH₂-c-Pr | CF₃ | H | Me | |
| 118 | Cl | H | H | CH₂-c-hexyl | Cl | H | Me | |
| 119 | Cl | H | H | CF₃ | CF₃ | H | Me | |
| 120 | Cl | H | H | CHFCH₂CH₃ | H | CF₃ | Me | |
| 121 | Cl | H | H | CHClCH₃ | CF₃ | H | Me | |
| 122 | Cl | H | H | CH₂OCH₃ | CN | H | Me | |
| 123 | Cl | H | H | CH₂OCH₂CH₃ | CF₃ | H | Me | |
| 124 | Cl | H | H | CF(CH₃)₂ | CF₃ | H | Me | |
| 125 | Cl | H | H | 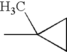 | Cl | H | Me | |
| 126 | Cl | H | Me | H | CF₃ | H | Me | |
| 127 | Cl | H | Me | Me | H | CF₃ | Me | |
| 128 | Cl | H | Me | Et | CF₃ | H | Me | |
| 129 | Cl | H | Me | Pr | CN | H | Me | |
| 130 | Cl | H | Me | i-Pr | CF₃ | H | Me | |
| 131 | Cl | H | Me | c-Pr | CF₃ | H | Me | |
| 132 | Cl | H | Me | Bu | Cl | H | Me | |
| 133 | Cl | H | Me | i-Bu | CF₃ | H | Me | |
| 134 | Cl | H | Me | c-Bu | H | CF₃ | Me | |
| 135 | Cl | H | Me | t-Bu | CF₃ | H | Me | |
| 136 | Cl | H | Me | c-hexyl | CN | H | Me | |
| 137 | Cl | H | Me | CH₂CH═CH₂ | CF₃ | H | Me | |
| 138 | Cl | H | Me | CH═CHCH₃ | CF₃ | H | Me | |
| 139 | Cl | H | Me | CH═CH₂ | Cl | H | Me | |
| 140 | Cl | H | Me | CH₂C≡CH | CF₃ | H | Me | |
| 141 | Cl | H | Me | C≡CCH₃ | H | CF₃ | Me | |
| 142 | Cl | H | Me | CH₂-c-Pr | CF₃ | H | Me | |
| 143 | Cl | H | Me | CH₂-c-hexyl | CN | H | Me | |
| 144 | Cl | H | Me | CF₃ | CF₃ | H | Me | |
| 145 | Cl | H | Me | CHFCH₂CH₃ | CF₃ | H | Me | |
| 146 | Cl | H | Me | CHClCH₃ | Cl | H | Me | |
| 147 | Cl | H | Me | CH₂OCH₃ | CF₃ | H | Me | |
| 148 | Cl | H | Me | CH₂OCH₂CH₃ | H | CF₃ | Me | |
| 149 | Cl | H | Me | CF(CH₃)₂ | CF₃ | H | Me | |
| 150 | Cl | H | Me | 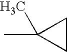 | CN | H | Me | |
| 151 | Cl | H | CO-Me | H | CF₃ | H | Me | |
| 152 | Cl | H | CO-Me | Me | CF₃ | H | Me | |
| 153 | Cl | H | CO-Me | Et | Cl | H | Me | |
| 154 | Cl | H | CO-Me | Pr | CF₃ | H | Me | |
| 155 | Cl | H | CO-Me | i-Pr | H | CF₃ | Me | |
| 156 | Cl | H | CO-Me | c-Pr | CF₃ | H | Me | |
| 157 | Cl | H | CO-Me | Bu | CN | H | Me | |
| 158 | Cl | H | CO-Me | i-Bu | CF₃ | H | Me | |
| 159 | Cl | H | CO-Me | c-Bu | CF₃ | H | Me | |
| 160 | Cl | H | CO-Me | t-Bu | Cl | H | Me | |
| 161 | Cl | H | CO-Me | c-hexyl | CF₃ | H | Me | |
| 162 | Cl | H | CO-Me | CH₂CH═CH₂ | H | CF₃ | Me | |
| 163 | Cl | H | CO-Me | CH═CHCH₃ | CF₃ | H | Me | |
| 164 | Cl | H | CO-Me | CH═CH₂ | CN | H | Me | |
| 165 | Cl | H | CO-Me | CH₂C≡CH | CF₃ | H | Me | |
| 166 | Cl | H | CO-Me | C≡CCH₃ | CF₃ | H | Me | |
| 167 | Cl | H | CO-Me | CH₂-c-Pr | Cl | H | Me | |
| 168 | Cl | H | CO-Me | CH₂-c-hexyl | CF₃ | H | Me | |
| 169 | Cl | H | CO-Me | CF₃ | H | CF₃ | Me | |
| 170 | Cl | H | CO-Me | CHFCH₂CH₃ | CF₃ | H | Me | |
| 171 | Cl | H | CO-Me | CHClCH₃ | CN | H | Me | |
| 172 | Cl | H | CO-Me | CH₂OCH₃ | CF₃ | H | Me | |
| 173 | Cl | H | CO-Me | CH₂OCH₂CH₃ | CF₃ | H | Me | |
| 174 | Cl | H | CO-Me | CF(CH₃)₂ | Cl | H | Me | |
| 175 | Cl | H | CO-Me | 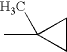 | CF₃ | H | Me | |
| 176 | Cl | H | CO-c-Pr | H | H | CF₃ | Me | |
| 177 | Cl | H | CO-c-Pr | Me | CF₃ | H | Me | |
| 178 | Cl | H | CO-c-Pr | Et | CN | H | Me | |
| 179 | Cl | H | CO-c-Pr | Pr | CF₃ | H | Me | |
| 180 | Cl | H | CO-c-Pr | i-Pr | CF₃ | H | Me | |
| 181 | Cl | H | CO-c-Pr | c-Pr | Cl | H | Me | |
| 182 | Cl | H | CO-c-Pr | Bu | CF₃ | H | Me | |
| 183 | Cl | H | CO-c-Pr | i-Bu | H | CF₃ | Me | |
| 184 | Cl | H | CO-c-Pr | c-Bu | CF₃ | H | Me | |
| 185 | Cl | H | CO-c-Pr | t-Bu | CN | H | Me | |
| 186 | Cl | H | CO-c-Pr | c-hexyl | CF₃ | H | Me | |
| 187 | Cl | H | CO-c-Pr | CH₂CH═CH₂ | CF₃ | H | Me | |
| 188 | Cl | H | CO-c-Pr | CH═CHCH₃ | Cl | H | Me | |
| 189 | Cl | H | CO-c-Pr | CH═CH₂ | CF₃ | H | Me | |
| 190 | Cl | H | CO-c-Pr | CH₂C≡CH | H | CF₃ | Me | |
| 191 | Cl | H | CO-c-Pr | C≡CCH₃ | CF₃ | H | Me | |
| 192 | Cl | H | CO-c-Pr | CH₂-c-Pr | CN | H | Me | |
| 193 | Cl | H | CO-c-Pr | CH₂-c-hexyl | CF₃ | H | Me | |
| 194 | Cl | H | CO-c-Pr | CF₃ | CF₃ | H | Me | |
| 195 | Cl | H | CO-c-Pr | CHFCH₂CH₃ | Cl | H | Me | |
| 196 | Cl | H | CO-c-Pr | CHClCH₃ | CF₃ | H | Me | |
| 197 | Cl | H | CO-c-Pr | CH₂OCH₃ | H | CF₃ | Me | |
| 198 | Cl | H | CO-c-Pr | CH₂OCH₂CH₃ | CF₃ | H | Me | |
| 199 | Cl | H | CO-c-Pr | CF(CH₃)₂ | CN | H | Me | |
| 200 | Cl | H | CO-c-Pr | 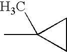 | CF₃ | H | Me | |
| 201 | Me | H | H | H | CF₃ | H | Me | |
| 202 | Me | H | H | Me | Cl | H | Me | |
| 203 | Me | H | H | Et | CF₃ | H | Me | |
| 204 | Me | H | H | i-Pr | CF₃ | H | Me | 6.80(s, 5-H, pyrimidine) |
| 205 | H | Me | H | i-Pr | CF₃ | H | Me | 8.50(s, 6-H, pyrimidine) |
| 206 | Me | H | H | c-Pr | CF₃ | H | Me | |
| 207 | Me | H | H | Bu | CF₃ | H | Me | |
| 208 | Me | H | H | i-Bu | CF₃ | H | Me | |
| 209 | Me | H | H | c-Bu | CF₃ | H | Me | 6.78(s, 5-H, pyrimidine) |
| 210 | Me | H | H | t-Bu | CF₃ | H | Me | |
| 211 | Me | H | H | c-hexyl | H | CF₃ | Me | |
| 212 | Me | H | H | CH₂CH═CH₂ | CF₃ | H | Me | |
| 213 | Me | H | H | CH═CHCH₃ | CN | H | Me | |
| 214 | Me | H | H | CH═CH₂ | CF₃ | H | Me | |
| 215 | Me | H | H | CH₂C≡CH | CF₃ | H | Me | |
| 216 | Me | H | H | C═CCH₃ | Cl | H | Me | |
| 217 | Me | H | H | CH₂-c-Pr | CF₃ | H | Me | |
| 218 | Me | H | H | CH₂-c-hexyl | H | CF₃ | Me | |
| 219 | Me | H | H | CF₃ | CF₃ | H | Me | 6.85(s, 5-H, pyrimidine) |
| 220 | Me | H | H | CF₃ | c-Pr | H | Me | 6.75(s, 5-H, pyrimidine) |
| 221 | Me | H | H | c-Pr | CF₂H | H | Me | 6.78(s, 5-H, pyrimidine) |
| 222 | Me | H | H | i-Pr | CF₂H | H | Me | 6.79(s, 5-H, pyrimidine) |
| 223 | Me | H | H | c-Pr | CF₃ | H | Et | 6.79(s, 5-H, pyrimidine) |
| 224 | Me | H | H | CF(CH₃)₂ | CF₃ | H | Me | |
| 225 | Me | H | H | 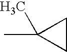 | H | CF₃ | Me | |
| 226 | Me | H | Me | H | CF₃ | H | Me | |
| 227 | Me | H | Me | Me | CN | H | Me | |
| 228 | Me | H | Me | Et | CF₃ | H | Me | |
| 229 | Me | H | Me | Pr | CF₃ | H | Me | |
| 230 | Me | H | Me | i-Pr | Cl | H | Me | |
| 231 | Me | H | Me | c-Pr | CF₃ | H | Me | |
| 232 | Me | H | Me | Bu | H | CF₃ | Me | |
| 233 | Me | H | Me | i-Bu | CF₃ | H | Me | |
| 234 | Me | H | Me | c-Bu | CN | H | Me | |
| 235 | Me | H | Me | t-Bu | CF₃ | H | Me | |
| 236 | Me | H | Me | c-hexyl | CF₃ | H | Me | |
| 237 | Me | H | Me | CH₂CH═CH₂ | Cl | H | Me | |
| 238 | Me | H | Me | CH═CHCH₃ | CF₃ | H | Me | |
| 239 | Me | H | Me | CH═CH₂ | H | CF₃ | Me | |
| 240 | Me | H | Me | CH₂C≡CH | CF₃ | H | Me | |
| 241 | Me | H | Me | C≡CCH₃ | CN | H | Me | |
| 242 | Me | H | Me | CH₂-c-Pr | CF₃ | H | Me | |
| 243 | Me | H | Me | CH₂-c-hexyl | CF₃ | H | Me | |
| 244 | Me | H | Me | CF₃ | Cl | H | Me | |
| 245 | Me | H | Me | CHFCH₂CH₃ | CF₃ | H | Me | |
| 246 | Me | H | Me | CHClCH₃ | H | CF₃ | Me | |
| 247 | Me | H | Me | CH₂OCH₃ | CF₃ | H | Me | |
| 248 | Me | H | Me | CH₂OCH₂CH₃ | CN | H | Me | |
| 249 | Me | H | Me | CF(CH₃)₂ | CF₃ | H | Me | |
| 250 | Me | H | Me | 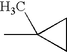 | CF₃ | H | Me | |
| 251 | Me | H | CO-Me | H | Cl | H | Me | |
| 252 | Me | H | CO-Me | Me | CF₃ | H | Me | |
| 253 | Me | H | CO-Me | Et | H | CF₃ | Me | |
| 254 | Me | H | CO-Me | Pr | CF₃ | H | Me | |
| 255 | Me | H | CO-Me | i-Pr | CN | H | Me | |
| 256 | Me | H | CO-Me | c-Pr | CF₃ | H | Me | |
| 257 | Me | H | CO-Me | Bu | CF₃ | H | Me | |
| 258 | Me | H | CO-Me | i-Bu | Cl | H | Me | |
| 259 | Me | H | CO-Me | c-Bu | CF₃ | H | Me | |
| 260 | Me | H | CO-Me | t-Bu | H | CF₃ | Me | |
| 261 | Me | H | CO-Me | c-hexyl | CF₃ | H | Me | |
| 262 | Me | H | CO-Me | CH₂CH═CH₂ | CN | H | Me | |
| 263 | Me | H | CO-Me | CH═CHCH₃ | CF₃ | H | Me | |
| 264 | Me | H | CO-Me | CH═CH₂ | CF₃ | H | Me | |
| 265 | Me | H | CO-Me | CH₂C≡CH | Cl | H | Me | |
| 266 | Me | H | CO-Me | C≡CCH₃ | CF₃ | H | Me | |
| 267 | Me | H | CO-Me | CH₂-c-Pr | H | CF₃ | Me | |
| 268 | Me | H | CO-Me | CH₂-c-hexyl | CF₃ | H | Me | |
| 269 | Me | H | CO-Me | CF₃ | CN | H | Me | |
| 270 | Me | H | CO-Me | CHFCH₂CH₃ | CF₃ | H | Me | |
| 271 | Me | H | CO-Me | CHClCH₃ | CF₃ | H | Me | |
| 272 | Me | H | CO-Me | CH₂OCH₃ | Cl | H | Me | |
| 273 | Me | H | CO-Me | CH₂OCH₂CH₃ | CF₃ | H | Me | |
| 274 | Me | H | CO-Me | CF(CH₃)₂ | H | CF₃ | Me | |
| 275 | Me | H | CO-Me | 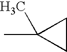 | CF₃ | H | Me | |
| 276 | Me | H | CO-c-Pr | H | CN | H | Me | |
| 277 | Me | H | CO-c-Pr | Me | CF₃ | H | Me | |
| 278 | Me | H | CO-c-Pr | Et | CF₃ | H | Me | |
| 279 | Me | H | CO-c-Pr | Pr | Cl | H | Me | |
| 280 | Me | H | CO-c-Pr | i-Pr | CF₃ | H | Me | |
| 281 | Me | H | CO-c-Pr | c-Pr | H | CF₃ | Me | |
| 282 | Me | H | CO-c-Pr | Bu | CF₃ | H | Me | |
| 283 | Me | H | CO-c-Pr | i-Bu | CN | H | Me | |
| 284 | Me | H | CO-c-Pr | c-Bu | CF₃ | H | Me | |
| 285 | Me | H | CO-c-Pr | t-Bu | CF₃ | H | Me | |
| 286 | Me | H | CO-c-Pr | c-hexyl | Cl | H | Me | |
| 287 | Me | H | CO-c-Pr | CH₂CH═CH₂ | CF₃ | H | Me | |
| 288 | Me | H | CO-c-Pr | CH═CHCH₃ | H | CF₃ | Me | |
| 289 | Me | H | CO-c-Pr | CH═CH₂ | CF₃ | H | Me | |
| 290 | Me | H | CO-c-Pr | CH₂C≡CH | CN | H | Me | |
| 291 | Me | H | CO-c-Pr | C≡CCH₃ | CF₃ | H | Me | |
| 292 | Me | H | CO-c-Pr | CH₂-c-Pr | CF₃ | H | Me | |
| 293 | Me | H | CO-c-Pr | CH₂-c-hexyl | Cl | H | Me | |
| 294 | Me | H | CO-c-Pr | CF₃ | CF₃ | H | Me | |
| 295 | Me | H | CO-c-Pr | CHFCH₂CH₃ | H | CF₃ | Me | |
| 296 | Me | H | CO-c-Pr | CHClCH₃ | CF₃ | H | Me | |
| 297 | Me | H | CO-c-Pr | CH₂OCH₃ | CN | H | Me | |
| 298 | Me | H | CO-c-Pr | CH₂OCH₂CH₃ | CF₃ | H | Me | |
| 299 | Me | H | CO-c-Pr | CF(CH₃)₂ | CF₃ | H | Me | |
| 300 | Me | H | CO-c-Pr | 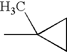 | Cl | H | Me | |
| 301 | Et | H | H | H | CF₃ | H | Me | |
| 302 | Et | H | H | Me | H | CF₃ | Me | |
| 303 | Et | H | H | Et | CF₃ | H | Me | |
| 304 | Et | H | H | Pr | CN | H | Me | |
| 305 | Et | H | H | i-Pr | CF₃ | H | Me | 6.79(s, 5-H, pyrimidine) |
| 306 | Et | H | H | c-Pr | CF₃ | H | Me | 6.82(s, 5-H, pyrimidine) |
| 307 | Et | H | H | Bu | Cl | Cl | Me | |
| 308 | Et | H | H | i-Bu | CF₃ | H | Me | |
| 309 | Et | H | H | c-Bu | H | CF₃ | Me | |
| 310 | Et | H | H | t-Bu | CF₃ | H | Me | 6.72(s, 5-H, pyrimidine) |
| 311 | Et | H | H | c-hexyl | CN | H | Me | |
| 312 | Et | H | H | c-Pr | OCF₂H | H | Me | 6.82(s, 5-H, pyrimidine) |
| 313 | Et | H | H | CH═CHCH₃ | CF₃ | H | Me | |
| 314 | Et | H | H | CH═CH₂ | Cl | H | Me | |
| 315 | Et | H | H | CH₂C≡CH | CF₃ | H | Me | |
| 316 | Et | H | H | C≡CCH₃ | H | CF₃ | Me | |
| 317 | Et | H | H | CH₂-c-Pr | CF₃ | H | Me | |
| 318 | Et | H | H | CH₂-c-hexyl | CN | H | Me | |
| 319 | Et | H | H | CF₃ | CF₃ | H | Me | 6.82(s, 5-H, pyrimidine) |
| 320 | Et | H | H | CHFCH₂CH₃ | CF₃ | H | Me | |
| 321 | Et | H | H | CHClCH₃ | Cl | H | Me | |
| 322 | Et | H | H | CH₂OCH₃ | CF₃ | H | Me | |
| 323 | Et | H | H | CH₂OCH₂CH₃ | H | CF₃ | Me | |
| 324 | Et | H | H | CF(CH₃)₂ | CF₃ | H | Me | 6.80(s, 5-H, pyrimidine) |
| 325 | Et | H | H | 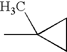 | CF₃ | H | Me | 6.80(s, 5-H, pyrimidine) |
| 326 | Et | H | H | 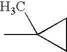 | c-Pr | H | Me | 6.78(s, 5-H, pyrimidine) |
| 327 | Et | H | H | 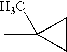 | OCF₂H | H | Me | 6.80(s, 5-H, pyrimidine) |
| 328 | Et | H | Me | Et | Cl | H | Me | |
| 329 | Et | H | Me | Pr | CF₃ | H | Me | |
| 330 | Et | H | Me | i-Pr | H | CF₃ | Me | |
| 331 | Et | H | Me | c-Pr | CF₃ | H | Me | |
| 332 | Et | H | Me | Bu | CN | H | Me | |
| 333 | Et | H | Me | i-Bu | CF₃ | H | Me | |
| 334 | Et | H | Me | c-Bu | CF₃ | H | Me | |
| 335 | Et | H | Me | t-Bu | Cl | H | Me | |
| 336 | Et | H | CO-Me | H | H | CF₃ | Me | |
| 337 | Et | H | CO-Me | Me | CF₃ | H | Me | |
| 338 | Et | H | CO-Me | Et | CN | H | Me | |
| 339 | Et | H | CO-Me | Pr | CF₃ | H | Me | |
| 340 | Et | H | CO-Me | i-Pr | CF₃ | H | Me | |
| 341 | Et | H | CO-Me | c-Pr | Cl | H | Me | |
| 342 | Et | H | CO-Me | Bu | CF₃ | H | Me | |
| 343 | Et | H | CO-Me | i-Bu | H | CF₃ | Me | |
| 344 | Et | H | CO-Me | c-Bu | CF₃ | H | Me | |
| 345 | Et | H | CO-Me | t-Bu | CN | H | Me | |
| 346 | Et | H | CO-Me | c-hexyl | CF₃ | H | Me | |
| 347 | Et | H | CO-Me | 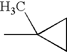 | CF₃ | H | Me | |
| 348 | Et | H | CO-c-Pr | H | CF₃ | H | Me | |
| 349 | Et | H | CO-c-Pr | Me | Cl | H | Me | |
| 350 | Et | H | CO-c-Pr | Et | CF₃ | H | Me | |
| 351 | Et | H | CO-c-Pr | Pr | H | CF₃ | Me | |
| 352 | Et | H | CO-c-Pr | i-Pr | CF₃ | H | Me | |
| 353 | Et | H | CO-c-Pr | c-Pr | CN | H | Me | |
| 354 | Et | H | CO-c-Pr | Bu | CF₃ | H | Me | |
| 355 | Et | H | CO-c-Pr | i-Bu | CF₃ | H | Me | |
| 356 | Et | H | CO-c-Pr | c-Bu | Cl | H | Me | |
| 357 | Et | H | CO-c-Pr | t-Bu | CF₃ | H | Me | |
| 358 | Et | H | CO-c-Pr | c-hexyl | H | CF₃ | Me | |
| 359 | OMe | H | H | H | CF₃ | H | Me | |
| 360 | OMe | H | H | Me | CN | H | Me | |
| 361 | OMe | H | H | Et | CF₃ | H | Me | |
| 362 | OMe | H | H | Pr | CF₃ | H | Me | |
| 363 | OMe | H | H | i-Pr | Cl | H | Me | |
| 364 | OMe | H | H | c-Pr | CF₃ | H | Me | |
| 365 | OMe | H | H | Bu | H | CF₃ | Me | |
| 366 | OMe | H | H | i-Bu | CF₃ | H | Me | |
| 367 | OMe | H | H | c-Bu | CN | H | Me | |
| 368 | OMe | H | H | t-Bu | CF₃ | H | Me | |
| 369 | OMe | H | H | c-hexyl | CF₃ | H | Me | |
| 370 | OMe | H | Me | H | Cl | H | Me | |
| 371 | OMe | H | Me | Me | CF₃ | H | Me | |
| 372 | OMe | H | Me | Et | H | CF₃ | Me | |
| 373 | OMe | H | Me | Pr | CF₃ | H | Me | |
| 374 | OMe | H | Me | i-Pr | CN | H | Me | |
| 375 | OMe | H | Me | c-Pr | CF₃ | H | Me | |
| 376 | OMe | H | Me | Bu | CF₃ | H | Me | |
| 377 | OMe | H | Me | i-Bu | Cl | H | Me | |
| 378 | OMe | H | Me | c-Bu | CF₃ | H | Me | |
| 379 | OMe | H | Me | t-Bu | H | CF₃ | Me | |
| 380 | OMe | H | CO-Me | H | CN | H | Me | |
| 381 | OMe | H | CO-Me | Me | CF₃ | H | Me | |
| 382 | OMe | H | CO-Me | Et | CF₃ | H | Me | |
| 383 | OMe | H | CO-Me | Pr | Cl | H | Me | |
| 384 | OMe | H | CO-Me | i-Pr | CF₃ | H | Me | |
| 385 | OMe | H | CO-Me | c-Pr | H | CF₃ | Me | |
| 386 | (CH₂)₃ | | H | c-Pr | H | CF₃ | Me | |
| 387 | OMe | H | H | c-Bu | CF₃ | H | Me | 6.20(s, 5-H, pyrimidine) |
| 388 | OMe | H | H | i-Bu | CF₃ | H | Me | 6.21(s, 5-H, pyrimidine) |
| 389 | OMe | H | H | Et | CF₃ | H | Me | 6.22(s, 5-H, pyrimidine) |
| 390 | OMe | H | H | c-Pr | CF₃ | H | Me | 6.21(s, 5-H, pyrimidine) |

TABLE 6
Compounds of the formula (I) according to the invention in which A is
A6 and X¹, X² are each hydrogen

| | |
|---|---|
| | (I) |
| 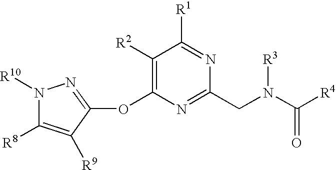 | |

| No. | R¹ | R² | R³ | R⁴ | R⁸ | R⁹ | R¹⁰ | ¹H-NMR: δ[CDCl₃] |
|---|---|---|---|---|---|---|---|---|
| 1 | H | H | H | H | H | CF₃ | Me | |
| 2 | H | H | H | Me | CF₃ | H | Me | |
| 3 | H | H | H | Et | CN | H | Me | |
| 4 | H | H | H | Pr | CF₃ | H | Me | |
| 5 | H | H | H | i-Pr | CF₃ | H | Me | |
| 6 | H | H | H | c-Pr | Cl | Cl | Me | |
| 7 | H | H | H | Bu | CF₃ | H | Me | |
| 8 | H | H | H | i-Bu | H | CF₃ | Me | |
| 9 | H | H | H | c-Bu | CF₃ | H | Me | |
| 10 | H | H | H | t-Bu | CN | H | Me | |
| 11 | H | H | H | c-hexyl | CF₃ | H | Me | |
| 12 | H | H | H | CH₂CH═CH₂ | CF₃ | H | Me | |
| 13 | H | H | H | CH═CHCH₃ | Cl | Cl | Me | |
| 14 | H | H | H | CH═CH₂ | CF₃ | H | Me | |
| 15 | H | H | H | CH₂C≡CH | H | CF₃ | Me | |
| 16 | H | H | H | C≡CCH₃ | CF₃ | H | Me | |
| 17 | H | H | H | CH₂-c-Pr | CN | H | Me | |
| 18 | H | H | H | CH₂-c-hexyl | CF₃ | H | Me | |
| 19 | H | H | H | CF₃ | CF₃ | H | Me | |
| 20 | H | H | H | CHFCH₂CH₃ | Cl | Cl | Me | |
| 21 | H | H | H | CHClCH₃ | CF₃ | H | Me | |
| 22 | H | H | H | CH₂OCH₃ | H | CF₃ | Me | |
| 23 | H | H | H | CH₂OCH₂CH₃ | CF₃ | H | Me | |
| 24 | H | H | H | CF(CH₃)₂ | CN | H | Me | |
| 25 | H | H | H | 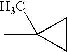 | CF₃ | H | Me | |
| 26 | H | H | Me | H | CF₃ | H | Me | |
| 27 | H | H | Me | Me | Cl | Cl | Me | |
| 28 | H | H | Me | Et | CF₃ | H | Me | |
| 29 | H | H | Me | Pr | H | CF₃ | Me | |
| 30 | H | H | Me | i-Pr | CF₃ | H | Me | |
| 31 | H | H | Me | c-Pr | CN | H | Me | |
| 32 | H | H | Me | Bu | CF₃ | H | Me | |
| 33 | H | H | Me | i-Bu | CF₃ | H | Me | |
| 34 | H | H | Me | c-Bu | Cl | Cl | Me | |
| 35 | H | H | Me | t-Bu | CF₃ | H | Me | |
| 36 | H | H | Me | c-hexyl | H | CF₃ | Me | |
| 37 | H | H | Me | CH₂CH═CH₂ | CF₃ | H | Me | |
| 38 | H | H | Me | CH═CHCH₃ | CN | H | Me | |
| 39 | H | H | Me | CH═CH₂ | CF₃ | H | Me | |
| 40 | H | H | Me | CH₂C≡CH | CF₃ | CF₃ | Me | |
| 41 | H | H | Me | C≡CCH₃ | Cl | H | Me | |
| 42 | H | H | Me | CH₂-c-Pr | CF₃ | H | Me | |
| 43 | H | H | Me | CH₂-c-hexyl | H | CF₃ | Me | |
| 44 | H | H | Me | CF₃ | CF₃ | H | Me | |
| 45 | H | H | Me | CHFCH₂CH₃ | CN | H | Me | |
| 46 | H | H | Me | CHClCH₃ | CF₃ | H | Me | |
| 47 | H | H | Me | CH₂OCH₃ | CF₃ | H | Me | |
| 48 | H | H | Me | CH₂OCH₂CH₃ | Cl | H | Me | |
| 49 | H | H | Me | CF(CH₃)₂ | CF₃ | H | Me | |
| 50 | H | H | Me | 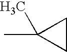 | H | CF₃ | Me | |
| 51 | H | H | CO-Me | H | CF₃ | H | Me | |
| 52 | H | H | CO-Me | Me | CN | H | Me | |
| 53 | H | H | CO-Me | Et | CF₃ | H | Me | |
| 54 | H | H | CO-Me | Pr | CF₃ | H | Me | |
| 55 | H | H | CO-Me | i-Pr | Cl | H | Me | |
| 56 | H | H | CO-Me | c-Pr | CF₃ | H | Me | |
| 57 | H | H | CO-Me | Bu | H | CF₃ | Me | |
| 58 | H | H | CO-Me | i-Bu | CF₃ | H | Me | |
| 59 | H | H | CO-Me | c-Bu | CN | H | Me | |
| 60 | H | H | CO-Me | t-Bu | CF₃ | H | Me | |
| 61 | H | H | CO-Me | c-hexyl | CF₃ | H | Me | |
| 62 | H | H | CO-Me | CH₂CH═CH₂ | Cl | H | Me | |
| 63 | H | H | CO-Me | CH═CHCH₃ | CF₃ | H | Me | |
| 64 | H | H | CO-Me | CH═CH₂ | H | CF₃ | Me | |
| 65 | H | H | CO-Me | CH₂C≡CH | CF₃ | H | Me | |
| 66 | H | H | CO-Me | C≡CCH₃ | CN | H | Me | |
| 67 | H | H | CO-Me | CH₂-c-Pr | CF₃ | H | Me | |
| 68 | H | H | CO-Me | CH₂-c-hexyl | CF₃ | H | Me | |
| 69 | H | H | CO-Me | CF₃ | Cl | H | Me | |
| 70 | H | H | CO-Me | CHFCH₂CH₃ | CF₃ | H | Me | |
| 71 | H | H | CO-Me | CHClCH₃ | H | CF₃ | Me | |
| 72 | H | H | CO-Me | CH₂OCH₃ | CF₃ | H | Me | |
| 73 | H | H | CO-Me | CH₂OCH₂CH₃ | CN | H | Me | |
| 74 | H | H | CO-Me | CF(CH₃)₂ | CF₃ | H | Me | |
| 75 | H | H | CO-Me | 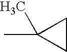 | CF₃ | H | Me | |
| 76 | H | H | CO-c-Pr | H | Cl | H | Me | |
| 77 | H | H | CO-c-Pr | Me | CF₃ | H | Me | |
| 78 | H | H | CO-c-Pr | Et | H | CF₃ | Me | |
| 79 | H | H | CO-c-Pr | Pr | CF₃ | H | Me | |
| 80 | H | H | CO-c-Pr | i-Pr | CN | H | Me | |
| 81 | H | H | CO-c-Pr | c-Pr | CF₃ | H | Me | |
| 82 | H | H | CO-c-Pr | Bu | CF₃ | H | Me | |
| 83 | H | H | CO-c-Pr | i-Bu | Cl | H | Me | |
| 84 | H | H | CO-c-Pr | c-Bu | CF₃ | H | Me | |
| 85 | H | H | CO-c-Pr | t-Bu | H | CF₃ | Me | |
| 86 | H | H | CO-c-Pr | c-hexyl | CF₃ | H | Me | |
| 87 | H | H | CO-c-Pr | CH₂CH═CH₂ | CN | H | Me | |
| 88 | H | H | CO-c-Pr | CH═CHCH₃ | CF₃ | H | Me | |
| 89 | H | H | CO-c-Pr | CH═CH₂ | CF₃ | H | Me | |
| 90 | H | H | CO-c-Pr | CH₂C═CH | Cl | H | Me | |
| 91 | H | H | CO-c-Pr | C≡CCH₃ | CF₃ | H | Me | |
| 92 | H | H | CO-c-Pr | CH₂-c-Pr | H | CF₃ | Me | |
| 93 | H | H | CO-c-Pr | CH₂-c-hexyl | CF₃ | H | Me | |
| 94 | H | H | CO-c-Pr | CF₃ | CN | H | Me | |
| 95 | H | H | CO-c-Pr | CHFCH₂CH₃ | CF₃ | H | Me | |
| 96 | H | H | CO-c-Pr | CHClCH₃ | CF₃ | H | Me | |
| 97 | H | H | CO-c-Pr | CH₂OCH₃ | Cl | H | Me | |
| 98 | H | H | CO-c-Pr | CH₂OCH₂CH₃ | CF₃ | H | Me | |
| 99 | H | H | CO-c-Pr | CF(CH₃)₂ | H | CF₃ | Me | |
| 100 | H | H | CO-c-Pr | 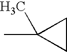 | CF₃ | H | Me | |
| 101 | Cl | H | H | H | CN | H | Me | |
| 102 | Cl | H | H | Me | CF₃ | H | Me | |
| 103 | Cl | H | H | Et | CF₃ | H | Me | |
| 104 | Cl | H | H | Pr | Cl | H | Me | |
| 105 | Cl | H | H | i-Pr | CF₃ | H | Me | |
| 106 | Cl | H | H | c-Pr | H | CF₃ | Me | |
| 107 | Cl | H | H | Bu | CF₃ | H | Me | |
| 108 | Cl | H | H | i-Bu | CN | H | Me | |
| 109 | Cl | H | H | c-Bu | CF₃ | H | Me | |
| 110 | Cl | H | H | t-Bu | CF₃ | H | Me | |
| 111 | Cl | H | H | c-hexyl | Cl | H | Me | |
| 112 | Cl | H | H | CH₂CH═CH₂ | CF₃ | H | Me | |
| 113 | Cl | H | H | CH═CHCH₃ | H | CF₃ | Me | |
| 114 | Cl | H | H | CH═CH₂ | CF₃ | H | Me | |
| 115 | Cl | H | H | CH₂C≡CH | CN | H | Me | |
| 116 | Cl | H | H | C≡CCH₃ | CF₃ | H | Me | |
| 117 | Cl | H | H | CH₂-c-Pr | CF₃ | H | Me | |
| 118 | Cl | H | H | CH₂-c-hexyl | Cl | H | Me | |
| 119 | Cl | H | H | CF₃ | CF₃ | H | Me | |
| 120 | Cl | H | H | CHFCH₂CH₃ | H | CF₃ | Me | |
| 121 | Cl | H | H | CHClCH₃ | CF₃ | H | Me | |
| 122 | Cl | H | H | CH₂OCH₃ | CN | H | Me | |
| 123 | Cl | H | H | CH₂OCH₂CH₃ | CF₃ | H | Me | |
| 124 | Cl | H | H | CF(CH₃)₂ | CF₃ | H | Me | |
| 125 | Cl | H | H | 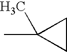 | Cl | H | Me | |
| 126 | Cl | H | Me | H | CF₃ | H | Me | |
| 127 | Cl | H | Me | Me | H | CF₃ | Me | |
| 128 | Cl | H | Me | Et | CF₃ | H | Me | |
| 129 | Cl | H | Me | Pr | CN | H | Me | |
| 130 | Cl | H | Me | i-Pr | CF₃ | H | Me | |
| 131 | Cl | H | Me | c-Pr | CF₃ | H | Me | |
| 132 | Cl | H | Me | Bu | Cl | H | Me | |
| 133 | Cl | H | Me | i-Bu | CF₃ | H | Me | |
| 134 | Cl | H | Me | c-Bu | H | CF₃ | Me | |
| 135 | Cl | H | Me | t-Bu | CF₃ | H | Me | |
| 136 | Cl | H | Me | c-hexyl | CN | H | Me | |
| 137 | Cl | H | Me | CH₂CH═CH₂ | CF₃ | H | Me | |
| 138 | Cl | H | Me | CHCHCH₃ | CF₃ | H | Me | |
| 139 | Cl | H | Me | CH═CH₂ | Cl | H | Me | |
| 140 | Cl | H | Me | CH₂C≡CH | CF₃ | H | Me | |
| 141 | Cl | H | Me | C≡CCH₃ | H | CF₃ | Me | |
| 142 | Cl | H | Me | CH₂-c-Pr | CF₃ | H | Me | |
| 143 | Cl | H | Me | CH₂-c-hexyl | CN | H | Me | |
| 144 | Cl | H | Me | CF₃ | CF₃ | H | Me | |
| 145 | Cl | H | Me | CHFCH₂CH₃ | CF₃ | H | Me | |
| 146 | Cl | H | Me | CHClCH₃ | Cl | H | Me | |
| 147 | Cl | H | Me | CH₂OCH₃ | CF₃ | H | Me | |
| 148 | Cl | H | Me | CH₂OCH₂CH₃ | H | CF₃ | Me | |
| 149 | Cl | H | Me | CF(CH₃)₂ | CF₃ | H | Me | |
| 150 | Cl | H | Me | 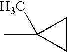 | CN | H | Me | |
| 151 | Cl | H | CO-Me | H | CF₃ | H | Me | |
| 152 | Cl | H | CO-Me | Me | CF₃ | H | Me | |
| 153 | Cl | H | CO-Me | Et | Cl | H | Me | |
| 154 | Cl | H | CO-Me | Pr | CF₃ | H | Me | |
| 155 | Cl | H | CO-Me | i-Pr | H | CF₃ | Me | |
| 156 | Cl | H | CO-Me | c-Pr | CF₃ | H | Me | |
| 157 | Cl | H | CO-Me | Bu | CN | H | Me | |
| 158 | Cl | H | CO-Me | i-Bu | CF₃ | H | Me | |
| 159 | Cl | H | CO-Me | c-Bu | CF₃ | H | Me | |
| 160 | Cl | H | CO-Me | t-Bu | Cl | H | Me | |
| 161 | Cl | H | CO-Me | c-hexyl | CF₃ | H | Me | |
| 162 | Cl | H | CO-Me | CH₂CH₂ | H | CF₃ | Me | |
| 163 | Cl | H | CO-Me | CH═CHCH₃ | CF₃ | H | Me | |
| 164 | Cl | H | CO-Me | CH═CH₂ | CN | H | Me | |
| 165 | Cl | H | CO-Me | CH₂C≡CH | CF₃ | H | Me | |
| 166 | Cl | H | CO-Me | C≡CCH₃ | CF₃ | H | Me | |
| 167 | Cl | H | CO-Me | CH₂-c-Pr | Cl | H | Me | |
| 168 | Cl | H | CO-Me | CH₂-c-hexyl | CF₃ | H | Me | |
| 169 | Cl | H | CO-Me | CF₃ | H | CF₃ | Me | |
| 170 | Cl | H | CO-Me | CHFCH₂CH₃ | CF₃ | H | Me | |
| 171 | Cl | H | CO-Me | CHClCH₃ | CN | H | Me | |
| 172 | Cl | H | CO-Me | CH₂OCH₃ | CF₃ | H | Me | |
| 173 | Cl | H | CO-Me | CH₂OCH₂CH₃ | CF₃ | H | Me | |
| 174 | Cl | H | CO-Me | CF(CH₃)₂ | Cl | H | Me | |
| 175 | Cl | H | CO-Me | 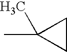 | CF₃ | H | Me | |
| 176 | Cl | H | CO-c-Pr | H | H | CF₃ | Me | |
| 177 | Cl | H | CO-c-Pr | Me | CF₃ | H | Me | |
| 178 | Cl | H | CO-c-Pr | Et | CN | H | Me | |
| 179 | Cl | H | CO-c-Pr | Pr | CF₃ | H | Me | |
| 180 | Cl | H | CO-c-Pr | i-Pr | CF₃ | H | Me | |
| 181 | Cl | H | CO-c-Pr | c-Pr | Cl | H | Me | |
| 182 | Cl | H | CO-c-Pr | Bu | CF₃ | H | Me | |
| 183 | Cl | H | CO-c-Pr | i-Bu | H | CF₃ | Me | |
| 184 | Cl | H | CO-c-Pr | c-Bu | CF₃ | H | Me | |
| 185 | Cl | H | CO-c-Pr | t-Bu | CN | H | Me | |
| 186 | Cl | H | CO-c-Pr | c-hexyl | CF₃ | H | Me | |
| 187 | Cl | H | CO-c-Pr | CH₂CH═CH₂ | CF₃ | H | Me | |
| 188 | Cl | H | CO-c-Pr | CH═CHCH₃ | Cl | H | Me | |
| 189 | Cl | H | CO-c-Pr | CH═CH₂ | CF₃ | H | Me | |
| 190 | Cl | H | CO-c-Pr | CH₂C≡CH | H | CF₃ | Me | |
| 191 | Cl | H | CO-c-Pr | C≡CCH₃ | CF₃ | H | Me | |
| 192 | Cl | H | CO-c-Pr | CH₂-c-Pr | CN | H | Me | |
| 193 | Cl | H | CO-c-Pr | CH₂-c-hexyl | CF₃ | H | Me | |
| 194 | Cl | H | CO-c-Pr | CF₃ | CF₃ | H | Me | |
| 195 | Cl | H | CO-c-Pr | CHFCH₂CH₃ | Cl | H | Me | |
| 196 | Cl | H | CO-c-Pr | CHClCH₃ | CF₃ | H | Me | |
| 197 | Cl | H | CO-c-Pr | CH₂OCH₃ | H | CF₃ | Me | |
| 198 | Cl | H | CO-c-Pr | CH₂OCH₂CH₃ | CF₃ | H | Me | |
| 199 | Cl | H | CO-c-Pr | CF(CH₃)₂ | CN | H | Me | |
| 200 | Cl | H | CO-c-Pr | 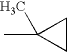 | CF₃ | H | Me | |
| 201 | Me | H | H | H | CF₃ | H | Me | |
| 202 | Me | H | H | Me | Cl | H | Me | |
| 203 | Me | H | H | Et | CF₃ | H | Me | |
| 204 | Me | H | H | i-Pr | CF₃ | H | Me | |
| 205 | H | Me | H | i-Pr | CF₃ | H | Me | |
| 206 | Me | H | H | c-Pr | CF₃ | H | Me | |
| 207 | Me | H | H | Bu | CF₃ | H | Me | |
| 208 | Me | H | H | i-Bu | CF₃ | H | Me | |
| 209 | Me | H | H | c-Bu | CF₃ | H | Me | |
| 210 | Me | H | H | t-Bu | CF₃ | H | Me | |
| 211 | Me | H | H | c-hexyl | H | CF₃ | Me | |
| 212 | Me | H | H | CH₂CHCH₂ | CF₃ | H | Me | |
| 213 | Me | H | H | CHCHCH₃ | CN | H | Me | |
| 214 | Me | H | H | CHCH₂ | CF₃ | H | Me | |
| 215 | Me | H | H | CH₂C≡CH | CF₃ | H | Me | |
| 216 | Me | H | H | C≡CCH₃ | Cl | H | Me | |
| 217 | Me | H | H | CH₂-c-Pr | CF₃ | H | Me | |
| 218 | Me | H | H | CH₂-c-hexyl | H | CF₃ | Me | |
| 219 | Me | H | H | CF₃ | CF₃ | H | Me | |
| 220 | Me | H | H | CF₃ | c-Pr | H | Me | |
| 221 | Me | H | H | c-Pr | CF₂H | H | Me | |
| 222 | Me | H | H | i-Pr | CF₂H | H | Me | |
| 223 | Me | H | H | c-Pr | CF₃ | H | Et | |
| 224 | Me | H | H | CF(CH₃)₂ | CF₃ | H | Me | |
| 225 | Me | H | H | 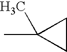 | H | CF₃ | Me | |
| 226 | Me | H | Me | H | CF₃ | H | Me | |
| 227 | Me | H | Me | Me | CN | H | Me | |
| 228 | Me | H | Me | Et | CF₃ | H | Me | |
| 229 | Me | H | Me | Pr | CF₃ | H | Me | |
| 230 | Me | H | Me | i-Pr | Cl | H | Me | |
| 231 | Me | H | Me | c-Pr | CF₃ | H | Me | |
| 232 | Me | H | Me | Bu | H | CF₃ | Me | |
| 233 | Me | H | Me | i-Bu | CF₃ | H | Me | |
| 234 | Me | H | Me | c-Bu | CN | H | Me | |
| 235 | Me | H | Me | t-Bu | CF₃ | H | Me | |
| 236 | Me | H | Me | c-hexyl | CF₃ | H | Me | |
| 237 | Me | H | Me | CH₂CHCH₂ | Cl | H | Me | |
| 238 | Me | H | Me | CH═CHCH₃ | CF₃ | H | Me | |
| 239 | Me | H | Me | CH═CH₂ | H | CF₃ | Me | |
| 240 | Me | H | Me | CH₂C≡CH | CF₃ | H | Me | |
| 241 | Me | H | Me | C≡CCH₃ | CN | H | Me | |
| 242 | Me | H | Me | CH₂-c-Pr | CF₃ | H | Me | |
| 243 | Me | H | Me | CH₂-c-hexyl | CF₃ | H | Me | |
| 244 | Me | H | Me | CF₃ | Cl | H | Me | |
| 245 | Me | H | Me | CHFCH₂CH₃ | CF₃ | H | Me | |
| 246 | Me | H | Me | CHClCH₃ | H | CF₃ | Me | |
| 247 | Me | H | Me | CH₂OCH₃ | CF₃ | H | Me | |
| 248 | Me | H | Me | CH₂OCH₂CH₃ | CN | H | Me | |
| 249 | Me | H | Me | CF(CH₃)₂ | CF₃ | H | Me | |
| 250 | Me | H | Me | 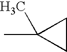 | CF₃ | H | Me | |
| 251 | Me | H | CO-Me | H | Cl | H | Me | |
| 252 | Me | H | CO-Me | Me | CF₃ | H | Me | |
| 253 | Me | H | CO-Me | Et | H | CF₃ | Me | |
| 254 | Me | H | CO-Me | Pr | CF₃ | H | Me | |
| 255 | Me | H | CO-Me | i-Pr | CN | H | Me | |
| 256 | Me | H | CO-Me | c-Pr | CF₃ | H | Me | |
| 257 | Me | H | CO-Me | Bu | CF₃ | H | Me | |
| 258 | Me | H | CO-Me | i-Bu | Cl | H | Me | |
| 259 | Me | H | CO-Me | c-Bu | CF₃ | H | Me | |
| 260 | Me | H | CO-Me | t-Bu | H | CF₃ | Me | |
| 261 | Me | H | CO-Me | c-hexyl | CF₃ | H | Me | |
| 262 | Me | H | CO-Me | CH₂CH═CH₂ | CN | H | Me | |
| 263 | Me | H | CO-Me | CH═CHCH₃ | CF₃ | H | Me | |
| 264 | Me | H | CO-Me | CHCH₂ | CF₃ | H | Me | |
| 265 | Me | H | CO-Me | CH₂C≡CH | Cl | H | Me | |
| 266 | Me | H | CO-Me | C≡CCH₃ | CF₃ | H | Me | |
| 267 | Me | H | CO-Me | CH₂-c-Pr | H | CF₃ | Me | |
| 268 | Me | H | CO-Me | CH₂-c-hexyl | CF₃ | H | Me | |
| 269 | Me | H | CO-Me | CF₃ | CN | H | Me | |
| 270 | Me | H | CO-Me | CHFCH₂CH₃ | CF₃ | H | Me | |
| 271 | Me | H | CO-Me | CHClCH₃ | CF₃ | H | Me | |
| 272 | Me | H | CO-Me | CH₂OCH₃ | Cl | H | Me | |
| 273 | Me | H | CO-Me | CH₂OCH₂CH₃ | CF₃ | H | Me | |
| 274 | Me | H | CO-Me | CF(CH₃)₂ | H | CF₃ | Me | |
| 275 | Me | H | CO-Me | 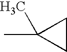 | CF₃ | H | Me | |
| 276 | Me | H | CO-c-Pr | H | CN | H | Me | |
| 277 | Me | H | CO-c-Pr | Me | CF₃ | H | Me | |
| 278 | Me | H | CO-c-Pr | Et | CF₃ | H | Me | |
| 279 | Me | H | CO-c-Pr | Pr | Cl | H | Me | |
| 280 | Me | H | CO-c-Pr | i-Pr | CF₃ | H | Me | |
| 281 | Me | H | CO-c-Pr | c-Pr | H | CF₃ | Me | |
| 282 | Me | H | CO-c-Pr | Bu | CF₃ | H | Me | |
| 283 | Me | H | CO-c-Pr | i-Bu | CN | H | Me | |
| 284 | Me | H | CO-c-Pr | c-Bu | CF₃ | H | Me | |
| 285 | Me | H | CO-c-Pr | t-Bu | CF₃ | H | Me | |
| 286 | Me | H | CO-c-Pr | c-hexyl | Cl | H | Me | |
| 287 | Me | H | CO-c-Pr | CH₂CH═CH₂ | CF₃ | H | Me | |
| 288 | Me | H | CO-c-Pr | CH═CHCH₃ | H | CF₃ | Me | |
| 289 | Me | H | CO-c-Pr | CH═CH₂ | CF₃ | H | Me | |
| 290 | Me | H | CO-c-Pr | CH₂C≡CH | CN | H | Me | |
| 291 | Me | H | CO-c-Pr | C≡CCH₃ | CF₃ | H | Me | |
| 292 | Me | H | CO-c-Pr | CH₂-c-Pr | CF₃ | H | Me | |
| 293 | Me | H | CO-c-Pr | CH₂-c-hexyl | Cl | H | Me | |
| 294 | Me | H | CO-c-Pr | CF₃ | CF₃ | H | Me | |
| 295 | Me | H | CO-c-Pr | CHFCH₂CH₃ | H | CF₃ | Me | |
| 296 | Me | H | CO-c-Pr | CHClCH₃ | CF₃ | H | Me | |
| 297 | Me | H | CO-c-Pr | CH₂OCH₃ | CN | H | Me | |
| 298 | Me | H | CO-c-Pr | CH₂OCH₂CH₃ | CF₃ | H | Me | |
| 299 | Me | H | CO-c-Pr | CF(CH₃)₂ | CF₃ | H | Me | |
| 300 | Me | H | CO-c-Pr | 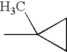 | Cl | H | Me | |
| 301 | Et | H | H | H | CF₃ | H | Me | |
| 302 | Et | H | H | Me | H | CF₃ | Me | |
| 303 | Et | H | H | Et | CF₃ | H | Me | |
| 304 | Et | H | H | Pr | CN | H | Me | |
| 305 | Et | H | H | i-Pr | CF₃ | H | Me | |
| 306 | Et | H | H | c-Pr | CF₃ | H | Me | 6.80 (s, 5-H, pyrimidine) |
| 307 | Et | H | H | Bu | Cl | Cl | Me | |
| 308 | Et | H | H | i-Bu | CF₃ | H | Me | |
| 309 | Et | H | H | c-Bu | H | CF₃ | Me | |
| 310 | Et | H | H | t-Bu | CF₃ | H | Me | 6.79 (s, 5-H, pyrimidine) |
| 311 | Et | H | H | c-hexyl | CN | H | Me | |
| 312 | Et | H | H | c-Pr | OCF₂H | H | Me | |
| 313 | Et | H | H | CH═CHCH₃ | CF₃ | H | Me | |
| 314 | Et | H | H | CH═CH₂ | Cl | H | Me | |
| 315 | Et | H | H | CH₂C≡CH | CF₃ | H | Me | |
| 316 | Et | H | H | C≡CCH₃ | H | CF₃ | Me | |
| 317 | Et | H | H | CH₂-c-Pr | CF₃ | H | Me | |
| 318 | Et | H | H | CH₂-c-hexyl | CN | H | Me | |
| 319 | Et | H | H | CF₃ | CF₃ | H | Me | |
| 320 | Et | H | H | CHFCH₂CH₃ | CF₃ | H | Me | |
| 321 | Et | H | H | CHClCH₃ | Cl | H | Me | |
| 322 | Et | H | H | CH₂OCH₃ | CF₃ | H | Me | |
| 323 | Et | H | H | CH₂OCH₂CH₃ | H | CF₃ | Me | |
| 324 | Et | H | H | CF(CH₃)₂ | CF₃ | H | Me | |
| 325 | Et | H | H | 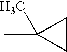 | CF₃ | H | Me | 6.70 (s, 5-H, pyrimidine) |
| 326 | Et | H | H | 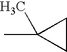 | c-Pr | H | Me | |
| 327 | Et | H | H | 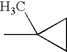 | OCF₂H | H | Me | |
| 328 | Et | H | Me | Et | Cl | H | Me | |
| 329 | Et | H | Me | Pr | CF₃ | H | Me | |
| 330 | Et | H | Me | i-Pr | H | CF₃ | Me | |
| 331 | Et | H | Me | c-Pr | CF₃ | H | Me | |
| 332 | Et | H | Me | Bu | CN | H | Me | |
| 333 | Et | H | Me | i-Bu | CF₃ | H | Me | |
| 334 | Et | H | Me | c-Bu | CF₃ | H | Me | |
| 335 | Et | H | Me | t-Bu | Cl | H | Me | |
| 336 | Et | H | Me | c-hexyl | CF₃ | H | Me | |
| 337 | OMe | H | Me | c-Pr | CF₃ | H | Me | |
| 338 | OMe | H | CO-Me | H | CF₃ | H | Me | |
| 339 | (CH₂)₃ | | Me | c-Pr | CF₃ | H | Me | |
| 340 | OMe | H | H | c-Bu | CF₃ | H | Me | 6.20 (s, 5-H, pyrimidine) |
| 341 | OMe | H | H | i-Bu | CF₃ | H | Me | 6.20 (s, 5-H, pyrimidine) |

### B. FORMULATION EXAMPLES

1. Dust

A dust is obtained by mixing 10 parts by weight of a compound of the formula (I) and 90 parts by weight of talc as inert substance and comminuting the mixture in a hammer mill.

2. Dispersible Powder

A wettable powder which is readily dispersible in water is obtained by mixing 25 parts by weight of a compound of the formula (I), 64 parts by weight of kaolin-containing quartz as inert substance, 10 parts by weight of potassium ligninsulfonate and 1 part by weight of sodium oleoylmethyltaurate as wetter and dispersant, and grinding the mixture in a pinned-disk mill.

3. Dispersion Concentrate

A dispersion concentrate which is readily dispersible in water is obtained by mixing 20 parts by weight of a compound of the formula (I), 6 parts by weight of alkylphenol polyglycol ether (®Triton X 207), 3 parts by weight of isotridecanol polyglycol ether (8 EO) and 71 parts by weight of paraffinic mineral oil (boiling range for example approx. 255 to above 277° C.), and grinding the mixture in a ball mill to a fineness of below 5 microns.

4. Emulsifiable Concentrate

An emulsifiable concentrate is obtained from 15 parts by weight of a compound of the formula (I), 75 parts by weight of cyclohexanone as solvent and 10 parts by weight of oxethylated nonylphenol as emulsifier.

5. Water-Dispersible Granules

Water-dispersible granules are obtained by mixing

```
75 parts by weight of a compound of the formula (I),
10 parts by weight of calcium ligninsulfonate,
  5 parts by weight of sodium lauryl sulfate,
  3 parts by weight of polyvinyl alcohol and
  7 parts by weight of kaolin,
```

grinding the mixture in a pinned-disk mill and granulating the powder in a fluidized bed by spraying on water as granulation liquid.

Water-dispersible granules are also obtained by homogenizing and precomminuting, in a colloid mill,

```
25 parts by weight of a compound of the formula (I),
  5 parts by weight of sodium 2,2′-dinaphthylmethane-6,6′-disulfonate,
  2 parts by weight of sodium oleoylmethyltaurate,
  1 parts by weight of polyvinyl alcohol,
17 parts by weight of calcium carbonate and
50 parts by weight of water,
```

subsequently grinding the mixture in a bead mill, and atomizing and drying the resulting suspension in a spray tower by means of a single-fluid nozzle.

### C. BIOLOGICAL EXAMPLES

1. Pre-Emergence Effect on Weeds

Seeds of monocotyledonous and dicotyledonous weed plants are placed into sandy loam soil in cardboard pots and covered with soil. The compounds of the invention, formulated as wettable powders or emulsion concentrates, are then applied to the surface of the soil cover in the form of aqueous suspensions or emulsions at an application rate of 600 to 800 l of water/ha (converted), in various dosages. After the treatment, the pots are placed in a greenhouse and kept under good growth conditions for the weeds. After the test plants have emerged, the damage to the plants or the negative effect on the emergence was scored visually after a test period of 3 to 4 weeks by comparison with untreated controls. After the test plants have remained in the greenhouse under optimum growth conditions for 3 to 4 weeks, the effect of the compounds is rated. Here, the compounds according to the invention have excellent activity against a broad spectrum of economically important monocotyledonous and dicotyledonous harmful plants, see Tables A to G.

2. Herbicidal Post-Emergence Effect on Harmful Plants

Seeds of monocotyledonous and dicotyledonous harmful plants are placed in sandy loam soil in cardboard pots, covered with soil and cultivated in a greenhouse under good growth conditions. Two to three weeks after sowing, the test plants are treated at the three-leaf stage. The compounds according to the invention, formulated in the form of wettable powders or emulsion concentrates, are sprayed onto the surface of the green parts of the plants at an application rate of 600 to 800 l of water/ha (converted), in various dosages. After the test plants have remained in the greenhouse under optimum growth conditions for 3 to 4 weeks, the effect of the compounds is rated. Here, the compounds according to the invention have excellent activity against a broad spectrum of economically important monocotyledonous and dicotyledonous harmful plants, see Tables H to J.

3. Crop Plant Compatibility

In further greenhouse experiments, seeds of barley and monocotyledonous and dicotyledonous harmful plants are placed in sandy loam soil, covered with soil and kept in a greenhouse until the plants have developed two to three true leaves. The treatment with the compounds of the formula (I) according to the invention is then carried out as described above under item 2. Visual scoring four to five weeks after the application and after the plants were kept in a greenhouse reveals that the compounds according to the invention are highly compatible with important crop plants, in particular wheat, corn and rice.

The abbreviations used in Tables A to J denote:

| | | | |
|---|---|---|---|
| AMARE | Amaranthus retroflexus | AVESA | Avena fatua |
| CYPIR | Cyperus iria | ECHCG | Echinochloa crus galli |
| LOLMU | Lolium multiflorum | SINAL | Sinapis arvensis |
| SETVI | Setaria viridis | STEME | Stellaria media |

TABLE A
Pre-emergence

| Compound | | Dosage | Herbicidal effect | | | |
|---|---|---|---|---|---|---|
| Table | Nr. | [g of a.i./ha] | AMARE | SETVI | LOLMU | STEME |
| 1 | 206 | 1000 | 100% | 100% | 100% | 100% |
| 1 | 505 | 1000 | 100% | 100% | 100% | 100% |

TABLE B
Pre-emergence

| Compound | | Dosage | Herbicidal effect | | | |
|---|---|---|---|---|---|---|
| Table | No. | [g of a.i./ha] | AMARE | SETVI | SINAL | STEME |
| 2 | 205 | 1000 | 100% | 100% | 90% | 100% |
| 2 | 304 | 1000 | 100% | 100% | 100% | 100% |

TABLE C
Pre-emergence

| Compound | | Dosage | Herbicidal effect | | | |
|---|---|---|---|---|---|---|
| Table | No. | [g of a.i./ha] | AMARE | AVESA | SETVI | SINAL |
| 3 | 306 | 1000 | 100% | 100% | 100% | 100% |

TABLE D
Pre-emergence

| Compound | | Dosage | Herbicidal effect | | | |
|---|---|---|---|---|---|---|
| Table | No. | [g of a.i./ha] | AMARE | SETVI | SINAL | STEME |
| 4 | 312 | 1000 | 100% | 100% | 100% | 100% |

TABLE E
Pre-emergence

| Compound | | Dosage | Herbicidal effect | | | |
|---|---|---|---|---|---|---|
| Table | No. | [g of a.i./ha] | LOLMU | SETVI | SINAL | STEME |
| 5 | 206 | 1000 | 100% | 100% | 100% | 100% |
| 5 | 319 | 1000 | 100% | 100% | 100% | 100% |

TABLE F
Pre-emergence

| Compound | | Dosage | Herbicidal effect | | | |
|---|---|---|---|---|---|---|
| Table | No. | [g of a.i./ha] | AMARE | SETVI | SINAL | STEME |
| 6 | 205 | 1000 | 100% | 100% | 100% | 100% |

TABLE G
Post-emergence

| Compound | | Dosage | Herbicidal effect | | | |
|---|---|---|---|---|---|---|
| Table | No. | [g of a.i./ha] | AMARE | SETVI | CYPIR | ECHCG |
| 2 | 205 | 1000 | 90% | 90% | 90% | 90% |

TABLE H
Post-emergence

| Compound | | Dosage | Herbicidal effect | | | |
|---|---|---|---|---|---|---|
| Table | No. | [g of a.i./ha] | AMARE | LOLMU | CYPIR | ECHCG |
| 3 | 306 | 1000 | 90% | 90% | 100% | 90% |

TABLE I
Post-emergence

| Compound | | Dosage | Herbicidal effect | | | |
|---|---|---|---|---|---|---|
| Table | No. | [g of a.i./ha] | AMARE | SINAL | STEME | ECHCG |
| 4 | 312 | 1000 | 90% | 90% | 100% | 100% |

TABLE J
Post-emergence

| Compound | | Dosage | Herbicidal effect | | | |
|---|---|---|---|---|---|---|
| Table | No. | [g of a.i./ha] | AMARE | LOLMU | CYPIR | ECHCG |
| 5 | 206 | 1000 | 90% | 90% | 100% | 100% |

1. A compound of the formula (I), its N-oxide and/or its salt, 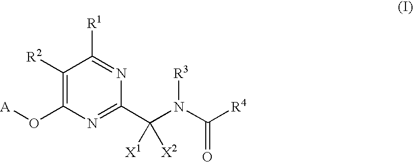
in which the radicals and indices are as defined below:
R¹ and R² independently of one another are hydrogen, halogen, cyano, amino, isocyanato, hydroxyl, nitro, COOR⁵, COR⁵, CH₂OH, CH₂SH, CH₂NH₂, (C₁-C₄)-alkyl, halo-(C₁-C₄)-alkyl, (C₃-C₆)-cycloalkyl, (C₁-C₄)-alkoxy, halo-(C₁-C₄)-alkoxy, (C₁-C₂)-alkoxy-(C₁-C₂)-alkyl, (C₂-C₄)-alkenyl, (C₂-C₄)-alkynyl, (C₃-C₄)-alkenyloxy, (C₃-C₄)-alkynyloxy, (C₁-C₂)-alkylthio-(C₁-C₂)-alkyl, S(O)ₙR⁶ (C₁-C₂)-alkylsulfonyl-(C₁-C₂)-alkyl, (C₁-C₄)-alkyl-NH, (C₁-C₃)-alkyl-CO—NH, (C₁-C₄)-alkyl-SO₂NH, di-(C₁-C₄)-alkylamino,
or R¹ and R² together form the group (CH₂)₃;
R³ is hydrogen, (C₁-C₄)-alkyl, (C₂-C₄)-alkenyl, (C₂-C₄)-alkynyl, benzyl, COOR⁵, COR⁴ or S(O)ₙR⁶;
R⁴ is hydrogen, (C₁-C₈)-alkyl, (C₂-C₈)-alkenyl, (C₂-C₈)-alkynyl, (C₃-C₆)-cycloalkyl, (C₃-C₆)-cycloalkyl which is substituted by one or two methyl groups, (C₁-C₂)-alkoxy-(C₁-C₂)-alkyl, (C₃-C₆)-cycloalkyl-(C₁-C₂)-alkyl, halo-(C₁-C₆)-alkyl or halo-(C₃-C₆)-cycloalkyl;
R⁵ is hydrogen or (C₁-C₄)-alkyl;
R⁶ is hydrogen, (C₁-C₄)-alkyl or halo-(C₁-C₄)-alkyl;
A is a radical from the group comprising the substituents A1 to A8 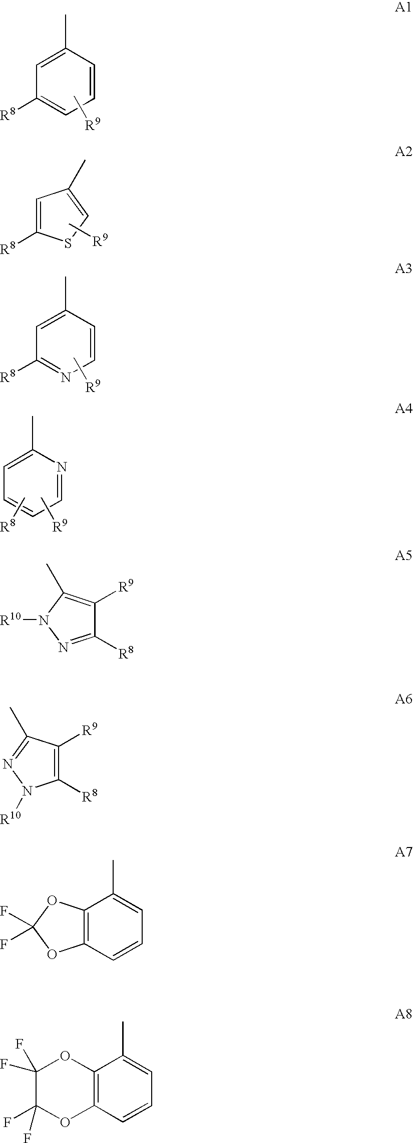
R⁸ is hydrogen, halogen, cyano, isocyanato, nitro, (C₁-C₄)-alkyl, halo-(C₁-C₄)-alkyl, (C₁-C₄)-alkoxy, halo-(C₁-C₄)-alkoxy, halo-(C₁-C₄)-alkylthio, (C₃-C₆)-cycloalkyl, halo-(C₃-C₆)-cycloalkyl, SF₅, S(O)ₙR⁶, (C₂-C₄)-alkenyl or (C₂-C₄)-alkynyl;
R⁹ is hydrogen, halogen, cyano, isocyanato, nitro, (C₁-C₄)-alkyl, halo-(C₁-C₄)-alkyl, (C₁-C₄)-alkoxy, halo-(C₁-C₄)-alkoxy, (C₂-C₄)-alkenyl, (C₂-C₄)-alkynyl, (C₃-C₆)-cycloalkyl or S(O)ₙR⁶;
R¹⁰ is (C₁-C₄)-alkyl;
X¹, X² independently of one another are hydrogen or (C₁-C₄)-alkyl;
n is 0, 1 or 2.

2. The compound as claimed in claim 1, in which
R¹ and R² independently of one another are hydrogen, halogen, cyano, hydroxyl, nitro, (C₁-C₂)-alkyl, halo-(C₁-C₂)-alkyl, (C₁-C₂)-alkoxy, halo-(C₁-C₂)-alkoxy, (C₁-C₂)-alkoxy-(C₁-C₂)-alkyl, (C₁-C₂)-alkylthio-(C₁-C₂)-alkyl, S(O)ₙ—(C₁-C₂)-alkyl,
or R¹ and R² together form the group (CH₂)₃;
R³ is hydrogen, (C₁-C₂)-alkyl, benzyl or COR⁴;
R⁴ is hydrogen, (C₁-C₆)-alkyl, (C₂-C₄)-alkenyl, (C₂-C₄)-alkynyl, (C₃-C₆)-cycloalkyl, (C₃-C₆)-cycloalkyl which is substituted by a methyl group, (C₁-C₂)-alkoxy-(C₁-C₂)-alkyl, (C₃-C₆)-cycloalkyl-(C₁-C₂)-alkyl, halo-(C₁-C₄)-alkyl or halo-(C₃-C₆)-cycloalkyl;
R⁵ is hydrogen or (C₁-C₄)-alkyl;
R⁶ is hydrogen, (C₁-C₂)-alkyl or halo-(C₁-C₂)-alkyl;
A is a radical from the group comprising the substituents A1 to A8;
R⁸ is hydrogen, halogen, cyano, (C₁-C₂)-alkyl, halo-(C₁-C₂)-alkyl, (C₁-C₂)-alkoxy, halo-(C₁-C₂)-alkoxy, halo-(C₁-C₂)-alkylthio, (C₃-C₆)-cycloalkyl, halo-(C₃-C₆)-cycloalkyl, S(O)ₙR⁶, (C₂-C₄)-alkenyl or (C₂-C₄)-alkynyl;
R⁹ is hydrogen, halogen, cyano, nitro, (C₁-C₂)-alkyl, halo-(C₁-C₂)-alkyl, (C₁-C₂)-alkoxy, halo-(C₁-C₂)-alkoxy, (C₂-C₂)-alkenyl, (C₂-C₄)-alkynyl, (C₃-C₆)-cycloalkyl or S(O)ₙR⁶;
R¹⁰ is methyl or ethyl;
X¹, X² independently of one another are hydrogen or methyl;
n is 0, 1 or 2.

3. The compound as claimed in claim 1, in which
R¹ and R² independently of one another are hydrogen, halogen, cyano, methyl, ethyl, trifluoromethyl, difluoromethyl, methoxy, trifluoromethoxy, difluoromethoxy, ethoxymethyl, methoxymethyl, thiomethyl, methylsulfonyl,
or R¹ and R² together form the group (CH₂)₃;
R³ is hydrogen, methyl, ethyl or COR⁴;
R⁴ is hydrogen, (C₁-C₄)-alkyl, (C₂-C₄)-alkenyl, (C₂-C₄)-alkynyl, (C₃-C₆)-cycloalkyl, cyclopropyl which is substituted by a methyl group, (C₁-C₂)-alkoxy-(C₁-C₂)-alkyl, (C₃-C₆)-cycloalkyl-(C₁-C₂)-alkyl, halo-(C₁-C₄)-alkyl or halo-(C₃-C₆)-cycloalkyl;
R⁵ is hydrogen or (C₁-C₄)-alkyl;
R⁶ is hydrogen, methyl or ethyl;
R⁷ is hydrogen, (C₁-C₄)-alkyl, (C₃-C₆)-cycloalkyl, (C₃-C₆)-cycloalkyl-(C₁-C₂)-alkyl, halo-(C₁-C₄)-alkyl or halo-(C₃-C₆)-cycloalkyl;
A is a radical from the group comprising the substituents A1 to A6;
R⁸ is hydrogen, halogen, cyano, methyl, ethyl, halo-(C₁-C₂)-alkyl, (C₁-C₂)-alkoxy, halomethoxy, (C₃-C₆)-cycloalkyl or S(O)ₙR⁶;
R⁹ is hydrogen, halogen, cyano, nitro, methyl, ethyl, halo-(C₁-C₂)-alkyl, (C₁-C₂)-alkoxy, halomethoxy, (C₂-C₂)-alkenyl, (C₂-C₄)-alkynyl, (C₃-C₆)-cycloalkyl or S(O)ₙR;
R¹⁰ is methyl or ethyl;
X¹, X² are hydrogen;
n is 0 or 2.

4. A herbicidal composition comprising a herbicidally effective amount of at least one compound of the formula (I) as claimed in claim 1.

5. The herbicidal composition as claimed in claim 4 as a mixture with formulating auxiliaries.

6. A method of controlling unwanted plants, which comprises applying to the plants or to the locus of unwanted plant growth an effective amount of a compound of the formula (I) as claimed in claim 1.

7. (canceled)

8. (canceled)

9. (canceled)

10. A method of controlling unwanted plants, which comprises applying to the plants or to the locus of unwanted plant growth an effective amount of a compound of the formula (I) as claimed in claim 4.

11. A method of controlling unwanted plants, which comprises applying to the plants or to the locus of unwanted plant growth an effective amount of a compound of the formula (I) as claimed in claim 5.

12. The method of claim 6, wherein the unwanted plants are present in crops of useful plants.

13. The method of claim 12, wherein the useful plants are transgenic useful plants.

14. The method of claim 10, wherein the unwanted plants are present in crops of useful plants.

15. The method of claim 14, wherein the useful plants are transgenic useful plants.

16. The method of claim 11, wherein the unwanted plants are present in crops of useful plants.

17. The method of claim 16, wherein the useful plants are transgenic useful plants.
